(19) United States
(12) Patent Application Publication
(10) Pub. No.: US 2023/0201144 A1
(43) Pub. Date: Jun. 29, 2023

(54) BIARYLSULFONAMIDES AND PHARMACEUTICAL COMPOSITIONS THEREOF, AND THEIR USE FOR TREATING FIBROTIC LUNG DISEASE

(71) Applicant: NovoMedix, LLC, San Diego, CA (US)

(72) Inventors: Cathy A. Swindlehurst, San Diego, CA (US); Kyle W.H. Chan, San Diego, CA (US); Laura Graciana Corral, La Jolla, CA (US); Paul E. Erdman, San Diego, CA (US); Leah M. Fung, San Diego, CA (US); Imelda Lam, San Diego, CA (US); Robert W. Sullivan, Vista, CA (US); Eduardo Torres, San Diego, CA (US)

(73) Assignee: NovoMedix, LLC, San Diego, CA (US)

(21) Appl. No.: 17/999,843

(22) PCT Filed: May 31, 2021
(86) PCT No.: PCT/US2021/035067

§ 371 Date: Nov. 23, 2022

Related U.S. Application Data
(60) Provisional application No. 63/032,361, filed on May 29, 2020.

(51) Int. Cl. A61K 31/18; A61K 31/4465; A61K 31/44

(52) U.S. Cl. CPC A61K 31/18; A61K 31/4465; A61K 31/44

(57) ABSTRACT

Provided herein are a biarylsulfonamide, e.g., a compound of Formula (IA), and a pharmaceutical composition thereof. Also provided herein is a method of treating a fibrotic lung disease with a biarylsulfonamide, alone or in combination with an antifibrotic agent. Additionally, provided herein is a method of slowing the rate of decline in pulmonary function in a subject having a fibrotic lung disease with a biarylsulfonamide, alone or in combination with an antifibrotic agent.

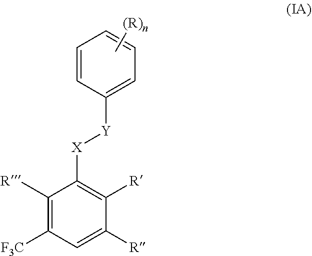

### CROSS REFERENCE TO RELATED APPLICATION

This application claims the benefit of the priority of U.S. Provisional Application No. 63/032,361, filed May 29, 2020; the disclosure of which is incorporated herein by reference in its entirety.

### FIELD

Provided herein are a biarylsulfonamide and a pharmaceutical composition thereof. Also provided herein is a method of treating a fibrotic lung disease with a biarylsulfonamide, alone or in combination with an antifibrotic agent. Additionally, provided herein is a method of slowing the rate of decline in pulmonary function in a subject having a fibrotic lung disease with a biarylsulfonamide, alone or in combination with an antifibrotic agent.

### BACKGROUND

Idiopathic pulmonary fibrosis (IPF) is a debilitating and fatal lung disease. Ley et. al., Am. J. Respir. Crit. Care Med. 2011, 183, 431-40; Blackwell et al., Am. J. Respir. Crit. Care Med. 2014, 189, 214-22. IPF causes permanent scarring of the lung, and the lung function of an IPF patient gradually and irreversibly declines over time. Barratt et al., J. Clin. Med. 2018, 7, E201. The prognosis for IPF is poor with a median survival after diagnosis of about 3 years. Blackwell et al., Am. J. Respir. Crit. Care Med. 2014, 189, 214-22.

IPF affects approximately 3 million people worldwide. Glassberg, Am. J. Manag. Care 2019, 25, S195-S203. In the United States, IPF affects approximately 130,000 people with about 50,000 new cases diagnosed annually, and as many as 40,000 Americans die from IPF every year. Blackwell et al., Am. J. Respir. Crit. Care Med. 2014, 189, 214-22. Globally, the incidence of IPF is rising with associated high morbidity, mortality, and economic healthcare burden. Hutchinson et al., Eur. Respir. J. 2015, 46, 604-6; Barratt et al., J. Clin. Med. 2018, 7, E201. IPF poses a growing threat to the global public health. Diamantopoulos et al., Pharmacoeconomics 2018, 36, 779-807.

The cause of IPF is unknown, but risk factors may include smoking, lung injury, family history of the disease, abnormal acid reflux, environmental exposures, and chronic viral infections. Ley et. al., Am. J. Respir. Crit. Care Med. 2011, 183, 431-40. The symptoms of IPF include a persistent dry and hacking cough, shortness of breath, chest discomfort, and finger clubbing. Id.

Despite the recent approvals of nintedanib and pirfenidone for IPF treatment, treatment options for IPF are limited. Raghu et. al., Am. J. Respir. Crit. Care Med. 2015, 192, 238-48. Therefore, there is a high unmet need for a safe and effective treatment that can alter the course of IPF by slowing disease progression.

### SUMMARY OF THE DISCLOSURE

Provided herein is a biarylsulfonamide of Formula (IA):

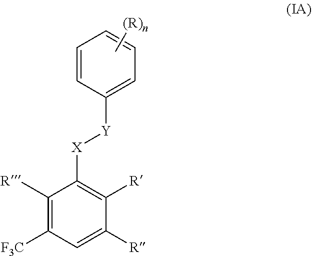

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
each R is independently deuterium, cyano, halo, nitro, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, heterocyclyl, —OR^{1a}, —C(O)OR^{1a}, —C(O)NR^{1b}R^{1c}, or —NR^{1b}R^{1c};
R′ and R″′ are each independently (i) hydrogen, deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c} or —S(O)₂NR^{1b}R^{1c};
R″ is (i) deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
each R^{1a}, R^{1b}, R^{1c}, and R^{1d} is independently hydrogen, deuterium, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or R^{1a} and R^{1c} together with the C and N atoms to which they are attached form heterocyclyl; or R^{1b} and R^{1c} together with the N atom to which they are attached form heterocyclyl;
X is —NH— and Y is —S(O)₂—; or X is —S(O)₂— and Y is —NH—; and
n is an integer of 2, 3, or 4;
wherein each alkyl, alkenyl, alkynyl, cycloalkyl, aryl, aralkyl, heteroaryl, and heterocyclyl is optionally substituted with one or more, in one embodiment, one, two, three, or four, substituents Q, wherein each Q is independently: (a) deuterium, cyano, halo, nitro, or oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is further optionally substituted with one or more, in one embodiment, one, two, three, or four, substituents Q^{a}; or (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, or —S(O)₂NR^{b}R^{c}, wherein each R^{a}, R^{b}, R^{c}, and R^{d} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl optionally substituted with one or more, in one embodiment, one, two, three, or four, substituents Q^{a};
wherein each Q^{a} is independently: (a) deuterium, cyano, halo, nitro, or oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{c})NR^{f}R^{g}, —OC(S)R^{e}, —OC(S)OR^{e}, —OC(S)NR^{f}R^{g}, —OS(O)R^{e}, —OS(O)₂R^{1c}, —OS(O)NR^{f}R^{g}, —OS(O)₂NR^{f}R^{g}, —NR^{f}R^{g}, —NR^{c}C(O)R^{h}, —NR^{c}C(O)OR^{f}, —NR^{c}C(O)NR^{f}R^{g}, —NR^{e}C(O)SR^{f}, —NR^{c}C(NR^{h})NR^{f}R^{g}, —NR^{e}C(S)R^{h}, —NR^{e}C(S)OR^{f}, —NR^{c}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{c}, —S(O)R^{1c}, —S(O)₂R^{1c}, —S(O)NR^{f}R^{g}, or —S(O)₂NR^{f}R^{g}; wherein each R^{c}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

Also provided herein is a pharmaceutical composition comprising a biarylsulfonamide of Formula (IA), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; and a pharmaceutically acceptable excipient.

Additionally provided herein is a method of treating, preventing, or ameliorating one or more symptoms of a fibrotic lung disease in a subject, comprising administering to the subject in need thereof a therapeutically effective amount of a biarylsulfonamide of Formula (IA), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

Furthermore, provided herein is a method of slowing the rate of decline in pulmonary function in a subject with a fibrotic lung disease, comprising administering to the subject in need thereof a therapeutically effective amount of a biarylsulfonamide of Formula (IA), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

Provided herein is a method of slowing the progression of a fibrotic lung disease in a subject, comprising administering to the subject in need thereof a therapeutically effective amount of a biarylsulfonamide of Formula (IA), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

Provided herein is a method of treating, preventing, or ameliorating one or more symptoms of a disorder, disease, or condition mediated by transforming growth factor-β (TFG-β) in a subject, comprising administering to a subject in need thereof a therapeutically effective amount of a biarylsulfonamide of Formula (IA), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

Provided herein is a method of inhibiting transforming growth factor-β (TFG-β) in a subject, comprising administering to the subject in need thereof a therapeutically effective amount of a biarylsulfonamide of Formula (IA), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

Provided herein is a method of inhibiting transforming growth factor-β (TFG-β) in a cell, comprising contacting the cell with an effective amount of a biarylsulfonamide of Formula (IA), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

Provided herein is a method of treating, preventing, or ameliorating one or more symptoms of a fibrotic lung disease in a subject, comprising administering to the subject in need thereof a therapeutically effective amount of a biarylsulfonamide and a therapeutically effective amount of an antifibrotic agent; wherein the biarylsulfonamide is a compound of Formula (IA):

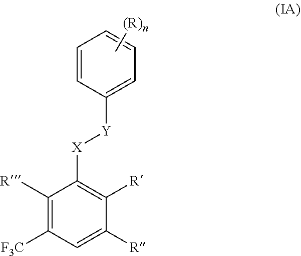

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
each R is independently deuterium, cyano, halo, nitro, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, heterocyclyl, or —OR^{1a};
R′ and R″′ are each independently (i) hydrogen, deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R″ is (i) deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c} or —S(O)₂NR^{1b}R^{1c};
each R^{1a}, R^{1b}, R^{1c}, and R^{1d} is independently hydrogen, deuterium, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or R^{1a} and R^{1c} together with the C and N atoms to which they are attached form heterocyclyl; or R^{1b} and R^{1c} together with the N atom to which they are attached form heterocyclyl;
X is —NH— and Y is —S(O)₂—; or X is —S(O)₂— and Y is —NH—; and
n is an integer of 2, 3, or 4;
wherein each alkyl, alkenyl, alkynyl, cycloalkyl, aryl, aralkyl, heteroaryl, and heterocyclyl is optionally substituted with one or more, in one embodiment, one, two, three, or four, substituents Q, wherein each Q is independently: (a) deuterium, cyano, halo, nitro, or oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is further optionally substituted with one or more, in one embodiment, one, two, three, or four, substituents Q^{a}; or (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, or —S(O)₂NR^{b}R^{c}, wherein each R^{a}, R^{b}, R^{c}, and R^{d} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl optionally substituted with one or more, in one embodiment, one, two, three, or four, substituents Q^{a};
wherein each Q^{a} is independently: (a) deuterium, cyano, halo, nitro, or oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; and (c) —C(O)R^{e}, —C(O)OR^{e}, —C(O)NR^{f}R^{g}, —C(O)SR^{e}, —C(NR^{c})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{c})NR^{f}R^{g}, —OC(S)R^{e}, —OC(S)OR^{e}, —OC(S)NR^{f}R^{g}, —OS(O)R^{e}, —OS(O)₂R^{1c}, —OS(O)NR^{f}R^{g}, —OS(O)₂NR′R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, or —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

Provided herein is a method of slowing the rate of decline in pulmonary function in a subject with a fibrotic lung disease, comprising administering to the subject in need thereof: (i) a therapeutically effective amount of a biarylsulfonamide of Formula (IA), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; and (ii) a therapeutically effective amount of an antifibrotic agent.

Provided herein is a method of slowing the progression of a fibrotic lung disease in a subject, comprising administering to the subject in need thereof: (i) a therapeutically effective amount of a biarylsulfonamide of Formula (IA), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; and (ii) a therapeutically effective amount of an antifibrotic agent.

Provided herein is a method of treating, preventing, or ameliorating one or more symptoms of a disorder, disease, or condition mediated by transforming growth factor-β (TFG-β) in a subject, comprising administering to a subject in need thereof: (i) a therapeutically effective amount of a biarylsulfonamide of Formula (IA), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; and (ii) a therapeutically effective amount of an antifibrotic agent.

Provided herein is a method of inhibiting transforming growth factor-β (TFG-β) in a subject, comprising administering to the subject in need thereof: (i) a therapeutically effective amount of a biarylsulfonamide of Formula (IA), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; and (ii) a therapeutically effective amount of an antifibrotic agent.

Provided herein is a method of inhibiting transforming growth factor-β (TFG-β) in a cell, comprising contacting the cell with (i) an effective amount of a biarylsulfonamide of Formula (IA), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; and (ii) an effective amount of an antifibrotic agent.

### DETAILED DESCRIPTION

To facilitate understanding of the disclosure set forth herein, a number of terms are defined below.

Generally, the nomenclature used herein and the laboratory procedures in organic chemistry, medicinal chemistry, biochemistry, biology, and pharmacology described herein are those well-known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject. In one embodiment, the subject is a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The terms “alleviate” and “alleviating” refer to easing or reducing one or more symptoms (e.g., pain) of a disorder, disease, or condition. The terms can also refer to reducing adverse effects associated with an active ingredient. Sometimes, the beneficial effects that a subject derives from a prophylactic or therapeutic agent do not result in a cure of the disorder, disease, or condition.

The term “contacting” or “contact” is meant to refer to bringing together of a therapeutic agent and a biological molecule (e.g., a protein, enzyme, RNA, or DNA), cell, or tissue such that a physiological and/or chemical effect takes place as a result of such contact. Contacting can take place in vitro, ex vivo, or in vivo. In one embodiment, a therapeutic agent is contacted with a biological molecule in vitro to determine the effect of the therapeutic agent on the biological molecule. In another embodiment, a therapeutic agent is contacted with a cell in cell culture (in vitro) to determine the effect of the therapeutic agent on the cell. In yet another embodiment, the contacting of a therapeutic agent with a biological molecule, cell, or tissue includes the administration of a therapeutic agent to a subject having the biological molecule, cell, or tissue to be contacted.

The term “therapeutically effective amount” or “effective amount” is meant to include the amount of a compound that, when administered, is sufficient to prevent development of, or alleviate to some extent, one or more of the symptoms of the disorder, disease, or condition being treated. The term “therapeutically effective amount” or “effective amount” also refers to the amount of a compound that is sufficient to elicit a biological or medical response of a biological molecule (e.g., a protein, enzyme, RNA, or DNA), cell, tissue, system, animal, or human, which is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of a subject (e.g., a human) without excessive toxicity, irritation, allergic response, immunogenicity, or other problems or complications, and commensurate with a reasonable benefit/risk ratio. See, e.g., Remington: The Science and Practice of Pharmacy, 23rd ed.; Adejare Ed.; Academic Press, 2020; Handbook of Pharmaceutical Excipients, 9th ed.; Sheskey et al., Eds.; Pharmaceutical Press, 2020; Handbook of Pharmaceutical Additives, 3rd ed.; Ash and Ash Eds.; Synapse Information Resources, 2007; Pharmaceutical Preformulation and Formulation, 1st ed.; Gibson Ed.; CRC Press, 2015.

The term “about” or “approximately” means an acceptable error for a particular value as determined by one of ordinary skill in the art, which depends in part on how the value is measured or determined. In certain embodiments, the term “about” or “approximately” means within 1, 2, or 3 standard deviations. In certain embodiments, the term “about” or “approximately” means within 25%, 20%, 15%, 10%, 9%, 8%, 7%, 6%, 5%, 4%, 3%, 2%, 1%, 0.5%, or 0.05% of a given value or range.

The term “alkyl” refers to a linear or branched saturated monovalent hydrocarbon radical, wherein the alkyl is optionally substituted with one or more substituents Q as described herein. For example, C₁₋₆ alkyl refers to a linear saturated monovalent hydrocarbon radical of 1 to 6 carbon atoms or a branched saturated monovalent hydrocarbon radical of 3 to 6 carbon atoms. In certain embodiments, the alkyl is a linear saturated monovalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkyl groups are also referred as “lower alkyl.” Examples of alkyl groups include, but are not limited to, methyl, ethyl, propyl (encompassing all isomeric forms, e.g., n-propyl and isopropyl), butyl (encompassing all isomeric forms, e.g., n-butyl, isobutyl, sec-butyl, and t-butyl), pentyl (encompassing all isomeric forms, e.g., n-pentyl, isopentyl, sec-pentyl, neopentyl, and tert-pentyl), and hexyl (encompassing all isomeric forms, e.g., n-hexyl, isohexyl, and sec-hexyl).

The term “alkenyl” refers to a linear or branched monovalent hydrocarbon radical, which contains one or more, in one embodiment, one, two, three, or four, in another embodiment, one, carbon-carbon double bond(s). The alkenyl is optionally substituted with one or more substituents Q as described herein. The term “alkenyl” embraces radicals having a “cis” or “trans” configuration or a mixture thereof, or alternatively, a “Z” or “E” configuration or a mixture thereof, as appreciated by those of ordinary skill in the art. For example, C₂₋₆ alkenyl refers to a linear unsaturated monovalent hydrocarbon radical of 2 to 6 carbon atoms or a branched unsaturated monovalent hydrocarbon radical of 3 to 6 carbon atoms. In certain embodiments, the alkenyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkenyl groups include, but are not limited to, ethenyl, propenyl (encompassing all isomeric forms, e.g., propen-1-yl, propen-2-yl, and allyl), and butenyl (encompassing all isomeric forms, e.g., buten-1-yl, buten-2-yl, buten-3-yl, and 2-buten-1-yl).

The term “alkynyl” refers to a linear or branched monovalent hydrocarbon radical, which contains one or more, in one embodiment, one, two, three, or four, in another embodiment, one, carbon-carbon triple bond(s). The alkynyl is optionally substituted with one or more substituents Q as described herein. For example, C₂₋₆ alkynyl refers to a linear unsaturated monovalent hydrocarbon radical of 2 to 6 carbon atoms or a branched unsaturated monovalent hydrocarbon radical of 4 to 6 carbon atoms. In certain embodiments, the alkynyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 4 to 20 (C₄-20), 4 to 15 (C₄₋₁₅), 4 to 10 (C₄₋₁₀), or 4 to 6 (C₄₋₆) carbon atoms. Examples of alkynyl groups include, but are not limited to, ethynyl (—C≡CH), propynyl (encompassing all isomeric forms, e.g., 1-propynyl (—C≡CCH₃) and propargyl (—CH₂C≡CH)), butynyl (encompassing all isomeric forms, e.g., 1-butyn-1-yl and 2-butyn-1-yl), pentynyl (encompassing all isomeric forms, e.g., 1-pentyn-1-yl and 1-methyl-2-butyn-1-yl), and hexynyl (encompassing all isomeric forms, e.g., 1-hexyn-1-yl and 2-hexyn-1-yl).

The term “cycloalkyl” refers to a cyclic monovalent hydrocarbon radical, which is optionally substituted with one or more substituents Q as described herein. In one embodiment, the cycloalkyl is a saturated or unsaturated but non-aromatic, and/or bridged or non-bridged, and/or fused bicyclic group. In certain embodiments, the cycloalkyl has from 3 to 20 (C₃₋₂₀), from 3 to 15 (C₃₋₁₅), from 3 to 10 (C₃₋₁₀), or from 3 to 7 (C₃₋₇) carbon atoms. In one embodiment, the cycloalkyl is monocyclic. In another embodiment, the cycloalkyl is bicyclic. In yet another embodiment, the cycloalkyl is tricyclic. In still another embodiment, the cycloalkyl is polycyclic. Examples of cycloalkyl groups include, but are not limited to, cyclopropyl, cyclobutyl, cyclopentyl, cyclopentenyl, cyclohexyl, cyclohexenyl, cyclohexadienyl, cycloheptyl, cycloheptenyl, bicyclo[1.1.1]pentyl, bicyclo[2.1.1]hexyl, bicyclo[2.2.1]heptyl, bicyclo[2.2.2]octyl, decalinyl, and adamantyl.

The term “aryl” refers to a monovalent monocyclic aromatic hydrocarbon radical and/or monovalent polycyclic aromatic hydrocarbon radical that contain at least one aromatic carbon ring. In certain embodiments, the aryl has from 6 to 20 (C₆₋₂₀), from 6 to 15 (C₆₋₁₅), or from 6 to 10 (C₆₋₁₀) ring carbon atoms. Examples of aryl groups include, but are not limited to, phenyl, naphthyl, fluorenyl, azulenyl, anthryl, phenanthryl, pyrenyl, biphenyl, and terphenyl. The aryl also refers to bicyclic or tricyclic carbon rings, where one of the rings is aromatic and the others of which may be saturated, partially unsaturated, or aromatic, for example, dihydronaphthyl, indenyl, indanyl, or tetrahydronaphthyl (tetralinyl). In one embodiment, the aryl is monocyclic. In another embodiment, the aryl is bicyclic. In yet another embodiment, the aryl is tricyclic. In still another embodiment, the aryl is polycyclic. In certain embodiments, the aryl is optionally substituted with one or more substituents Q as described herein.

The term “aralkyl” or “arylalkyl” refers to a monovalent alkyl group substituted with one or more aryl groups. In certain embodiments, the aralkyl has from 7 to 30 (C₇₋₃₀), from 7 to 20 (C₇-20), or from 7 to 16 (C₇₋₁₆) carbon atoms. Examples of aralkyl groups include, but are not limited to, benzyl, phenylethyl (including all isomeric forms, e.g., 1-phenylethyl and 2-phenylethyl), and phenylpropyl (encompassing all isomeric forms, e.g., 1-phenylpropyl, 2-phenylpropyl, and 3-phenylpropyl). In certain embodiments, the aralkyl is optionally substituted with one or more substituents Q as described herein.

The term “heteroaryl” refers to a monovalent monocyclic aromatic group or monovalent polycyclic aromatic group that contain at least one aromatic ring, wherein at least one aromatic ring contains one or more heteroatoms, each independently selected from O, S, and N, in the ring. The heteroaryl is bonded to the rest of a molecule through the aromatic ring. Each ring of a heteroaryl group can contain one or two O atoms, one or two S atoms, and/or one to four N atoms; provided that the total number of heteroatoms in each ring is four or less and each ring contains at least one carbon atom. In certain embodiments, the heteroaryl has from 5 to 20, from 5 to 15, or from 5 to 10 ring atoms. In one embodiment, the heteroaryl is monocyclic. Examples of monocyclic heteroaryl groups include, but are not limited to, furanyl, imidazolyl, isothiazolyl, isoxazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl. In another embodiment, the heteroaryl is bicyclic. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyridinyl, imidazopyridinyl, imidazothiazolyl, indolizinyl, indolyl, indazolyl, isobenzofuranyl, isobenzothienyl, isoindolyl, isoquinolinyl, isothiazolyl, naphthyridinyl, oxazolopyridinyl, phthalazinyl, pteridinyl, purinyl, pyridopyridyl, pyrrolopyridyl, quinolinyl, quinoxalinyl, quinazolinyl, thiadiazolopyrimidyl, and thienopyridyl. In yet another embodiment, the heteroaryl is tricyclic. Examples of tricyclic heteroaryl groups include, but are not limited to, acridinyl, benzindolyl, carbazolyl, dibenzofuranyl, perimidinyl, phenanthrolinyl, phenanthridinyl, phenarsazinyl, phenazinyl, phenothiazinyl, phenoxazinyl, and xanthenyl. In certain embodiments, the heteroaryl is optionally substituted with one or more substituents Q as described herein.

The term “heterocyclyl” or “heterocyclic” refers to a monovalent monocyclic non-aromatic ring system or monovalent polycyclic ring system that contains at least one non-aromatic ring, wherein one or more of the non-aromatic ring atoms are heteroatoms, each independently selected from O, S, and N; and the remaining ring atoms are carbon atoms. In certain embodiments, the heterocyclyl or heterocyclic group has from 3 to 20, from 3 to 15, from 3 to 10, from 3 to 8, from 4 to 7, or from 5 to 6 ring atoms. The heterocyclyl is bonded to the rest of a molecule through the non-aromatic ring. In certain embodiments, the heterocyclyl is a monocyclic, bicyclic, tricyclic, or tetracyclic ring system, which may be fused or bridged, and in which nitrogen or sulfur atoms may be optionally oxidized, nitrogen atoms may be optionally quaternized, and some rings may be partially or fully saturated, or aromatic. The heterocyclyl may be attached to the main structure at any heteroatom or carbon atom which results in the creation of a stable compound. Examples of heterocyclyls and heterocyclic groups include, but are not limited to, azepinyl, benzodioxanyl, benzodioxolyl, benzofuranonyl, benzopyranonyl, benzopyranyl, benzotetrahydrofuranyl, benzotetrahydrothienyl, benzothiopyranyl, benzoxazinyl, O-carbolinyl, chromanyl, chromonyl, cinnolinyl, coumarinyl, decahydroisoquinolinyl, dihydrobenzisothiazinyl, dihydrobenzisoxazinyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, imidazolinyl, indolinyl, isobenzotetrahydrofuranyl, isobenzotetrahydrothienyl, isochromanyl, isocoumarinyl, isoindolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, tetrahydroquinolinyl, and 1,3,5-trithianyl. In certain embodiments, the heterocyclyl is optionally substituted with one or more substituents Q as described herein.

The term “halogen”, “halide,” or “halo” refers to fluoro, chloro, bromo, and/or iodo.

The term “optionally substituted” is intended to mean that a group or substituent, such as an alkyl, alkenyl, alkynyl, cycloalkyl, aryl, aralkyl, heteroaryl, or heterocyclyl group, may be substituted with one or more, in one embodiment, one, two, three, or four, substituents Q, each of which is independently, e.g., (a) deuterium (-D), cyano (—CN), halo, nitro (—NO₂), or oxo (═O); (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is further optionally substituted with one or more, in one embodiment, one, two, three, or four, substituents Q^{a}; or (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, or —S(O)₂NR^{b}R^{c}, wherein each R^{a}, R^{b}, R^{c}, and R^{d} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl optionally substituted with one or more, in one embodiment, one, two, three, or four, substituents Q^{a}. As used herein, all groups that can be substituted are “optionally substituted.”

In one embodiment, each Q^{a} is independently: (a) deuterium, cyano, halo, nitro, or oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{e}, —C(O)OR^{e}, —C(O)NR^{f}R^{g}, —C(O)SR^{e}, —C(NR^{c})NR^{f}R^{g}, —C(S)R^{c}, —C(S)OR^{c}, —C(S)NR^{f}R^{g}, —OR^{c}, —OC(O)R^{c}, —OC(O)OR^{c}, —OC(O)NR^{f}R^{g}, —OC(O)SR^{c}, —OC(NR^{c})NR^{f}R^{g}, —OC(S)R^{c}, —OC(S)OR^{c}, —OC(S)NR^{f}R^{g}, —OS(O)R^{e}, —OS(O)₂R^{1c}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{c}C(O)NR^{f}R^{g}, —NR^{e}C(O)SR^{f}, —NR^{c}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{c}, —S(O)R^{e}, —S(O)₂R^{1c}, —S(O)NR^{f}R^{g}, or —S(O)₂NR^{f}R^{g}; wherein each R^{c}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In certain embodiments, “optically active” and “enantiomerically active” refer to a collection of molecules, which has an enantiomeric excess of no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, an optically active compound comprises about 95% or more of one enantiomer and about 5% or less of the other enantiomer based on the total weight of the enantiomeric mixture in question. In certain embodiments, an optically active compound comprises about 98% or more of one enantiomer and about 2% or less of the other enantiomer based on the total weight of the enantiomeric mixture in question. In certain embodiments, an optically active compound comprises about 99% or more of one enantiomer and about 1% or less of the other enantiomer based on the total weight of the enantiomeric mixture in question.

In describing an optically active compound, the prefixes R and S are used to denote the absolute configuration of the compound about its chiral center(s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the compound, R and S.

The term “isotopically enriched” refers to a compound that contains an unnatural proportion of an isotope at one or more of the atoms that constitute such a compound. In certain embodiments, an isotopically enriched compound contains unnatural proportions of one or more isotopes, including, but not limited to, hydrogen (¹H), deuterium (2H), tritium (3H), carbon-11 (¹¹C), carbon-12 (¹²C), carbon-13 (¹³C), carbon-14 (¹⁴C), nitrogen-13 (¹³N), nitrogen-14 (14N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I), iodine-125 (¹²⁵I), iodine-127 (¹²⁷I), iodine-129 (¹²⁹I), and iodine-131 (¹³¹I). In certain embodiments, an isotopically enriched compound is in a stable form, that is, non-radioactive. In certain embodiments, an isotopically enriched compound contains unnatural proportions of one or more isotopes, including, but not limited to, hydrogen (¹H), deuterium (2H), carbon-12 (¹²C), carbon-13 (¹³C), nitrogen-14 (14N), nitrogen-15 (¹⁵N), oxygen-16 (¹⁶O), oxygen-17 (¹⁷O), oxygen-18 (¹⁸O), fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, an isotopically enriched compound is in an unstable form, that is, radioactive. In certain embodiments, an isotopically enriched compound contains unnatural proportions of one or more isotopes, including, but not limited to, tritium (3H), carbon-11 (¹¹C), carbon-14 (¹⁴C), nitrogen-13 (¹³N), oxygen-14 (¹⁴O), oxygen-15 (¹⁵O), fluorine-18 (¹⁸F), phosphorus-32 (³²P), phosphorus-33 (³³P), sulfur-35 (³SS), chlorine-36 (³⁶Cl), iodine-123 (¹²³I), iodine-125 (¹²⁵I), iodine-129 (¹²⁹I), and iodine-131 (¹³¹I). It will be understood that, in a compound as provided herein, any hydrogen can be ²H, as example, or any carbon can be ¹³C, as example, or any nitrogen can be ¹⁵N, as example, or any oxygen can be ¹⁸O, as example, where feasible according to the judgment of one of ordinary skill in the art.

The term “isotopic enrichment” refers to the percentage of incorporation of a less prevalent isotope (e.g., D for deuterium or hydrogen-2) of an element at a given position in a molecule in the place of a more prevalent isotope (e.g., ¹H for protium or hydrogen-1) of the element. As used herein, when an atom at a particular position in a molecule is designated as a particular less prevalent isotope, it is understood that the abundance of that isotope at that position is substantially greater than its natural abundance.

The term “isotopic enrichment factor” refers the ratio between the isotopic abundance in an isotopically enriched compound and the natural abundance of a specific isotope.

The term “hydrogen” or the symbol “H” refers to the composition of naturally occurring hydrogen isotopes, which include protium (¹H), deuterium (²H or D), and tritium (3H), in their natural abundances. Protium is the most common hydrogen isotope having a natural abundance of more than 99.98%. Deuterium is a less prevalent hydrogen isotope having a natural abundance of about 0.0156%.

The term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156% on average, deuterium enrichment at any position in a compound synthesized using non-enriched starting materials is about 0.0156% on average. As used herein, when a particular position in an isotopically enriched compound is designated as having deuterium, it is understood that the abundance of deuterium at that position in the compound is substantially greater than its natural abundance (0.0156%).

The term “carbon” or the symbol “C” refers to the composition of naturally occurring carbon isotopes, which include carbon-12 (¹²C) and carbon-13 (¹³C) in their natural abundances. Carbon-12 is the most common carbon isotope having a natural abundance of more than 98.89%. Carbon-13 is a less prevalent carbon isotope having a natural abundance of about 1.11%.

The term “carbon-13 enrichment” or “¹³C enrichment” refers to the percentage of incorporation of carbon-13 at a given position in a molecule in the place of carbon. For example, carbon-13 enrichment of 10% at a given position means that 10% of molecules in a given sample contain carbon-13 at the specified position. Because the naturally occurring distribution of carbon-13 is about 1.11% on average, carbon-13 enrichment at any position in a compound synthesized using non-enriched starting materials is about 1.11% on average. As used herein, when a particular position in an isotopically enriched compound is designated as having carbon-13, it is understood that the abundance of carbon-13 at that position in the compound is substantially greater than its natural abundance (1.11%).

The terms “substantially pure” and “substantially homogeneous” mean sufficiently homogeneous to appear free of readily detectable impurities as determined by standard analytical methods used by one of ordinary skill in the art, including, but not limited to, thin layer chromatography (TLC), gel electrophoresis, high performance liquid chromatography (HPLC), gas chromatography (GC), nuclear magnetic resonance (NMR), and mass spectrometry (MS); or sufficiently pure such that further purification would not detectably alter the physical, chemical, biological, and/or pharmacological properties, such as enzymatic and biological activities, of the substance. In certain embodiments, “substantially pure” or “substantially homogeneous” refers to a collection of molecules, wherein at least about 95%, at least about 96%, at least about 97%, at least about 98%, at least about 99%, or at least about 99.5% by weight of the molecules are a single compound, including a single enantiomer, a racemic mixture, or a mixture of enantiomers, as determined by standard analytical methods. As used herein, when an atom at a particular position in an isotopically enriched molecule is designated as a particular less prevalent isotope, a molecule that contains other than the designated isotope at the specified position is an impurity with respect to the isotopically enriched compound. Thus, for a deuterated compound that has an atom at a particular position designated as deuterium, a compound that contains a protium at the same position is an impurity.

The term “solvate” refers to a complex or aggregate formed by one or more molecules of a solute, e.g., a compound provided herein, and one or more molecules of a solvent, which are present in a stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

The phrase “an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof” has the same meaning as the phrase “(i) an enantiomer, a mixture of enantiomers, a diastereomer, a mixture of two or more diastereomers, a tautomer, a mixture of two or more tautomers, or an isotopic variant of the compound referenced therein; or (ii) a pharmaceutically acceptable salt, solvate, hydrate, or prodrug of the compound referenced therein; or (iii) a pharmaceutically acceptable salt, solvate, hydrate, or prodrug of an enantiomer, a mixture of enantiomers, a diastereomer, a mixture of two or more diastereomers, a tautomer, a mixture of two or more tautomers, or an isotopic variant of the compound referenced therein.”

### Biarylsulfonamides

In one embodiment, provided herein is a biarylsulfonamide of Formula (IA):

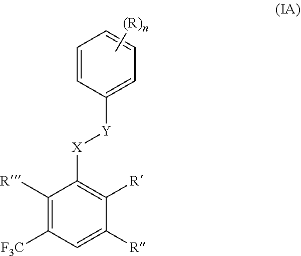

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
each R is independently deuterium, cyano, halo, nitro, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, heterocyclyl, —OR^{1a}, —C(O)OR^{1a}, —C(O)NR^{1b}R^{1c}, or —NR^{1b}R^{1c};
R′ and R″′ are each independently (i) hydrogen, deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c} or —S(O)₂NR^{1b}R^{1c};
R″ is (i) deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
each R^{1a}, R^{1b}, R^{1c}, and R^{1d} is independently hydrogen, deuterium, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or R^{1a} and R^{1c} together with the C and N atoms to which they are attached form heterocyclyl; or R^{1b} and R^{1c} together with the N atom to which they are attached form heterocyclyl;
X is —NH— and Y is —S(O)₂—; or X is —S(O)₂— and Y is —NH—; and
n is an integer of 2, 3, or 4;
wherein each alkyl, alkenyl, alkynyl, cycloalkyl, aryl, aralkyl, heteroaryl, and heterocyclyl is optionally substituted with one or more, in one embodiment, one, two, three, or four, substituents Q, wherein each Q is independently: (a) deuterium, cyano, halo, nitro, or oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is further optionally substituted with one or more, in one embodiment, one, two, three, or four, substituents Q^{a}; or (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, or —S(O)₂NR^{b}R^{c}, wherein each R^{a}, R^{b}, R^{c}, and R^{d} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl optionally substituted with one or more, in one embodiment, one, two, three, or four, substituents Q^{a};
wherein each Q^{a} is independently: (a) deuterium, cyano, halo, nitro, or oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; and (c) —C(O)R^{e}, —C(O)OR^{e}, —C(O)NR^{f}R^{g}, —C(O)SR^{e}, —C(NR^{c})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{c})NR^{f}R^{g}, —OC(S)R^{e}, —OC(S)OR^{e}, —OC(S)NR^{f}R^{g}, —OS(O)R^{e}, —OS(O)₂R^{1c}, —OS(O)NR^{f}R^{g}, —OS(O)₂NR^{f}R^{g}, —NR^{f}R^{g}, —NR^{c}C(O)R^{h}, —NR^{c}C(O)OR^{f}, —NR^{c}C(O)NR^{f}R^{g}, —NR^{e}C(O)SR^{f}, —NR^{c}C(NR^{h})NR^{f}R^{g}, —NR^{e}C(S)R^{h}, —NR^{e}C(S)OR^{f}, —NR^{c}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{c}, —S(O)R^{1c}, —S(O)₂R^{1c}, —S(O)NR^{f}R^{g}, or —S(O)₂NR^{f}R^{g}; wherein each R^{c}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In one embodiment, in Formula IA, each R is independently (i) deuterium, cyano, halo, or nitro; or (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, heterocyclyl, or —OR^{1a}, each of which is optionally substituted with one or more substituents Q; wherein R^{1a} is as defined herein. In another embodiment, in Formula IA, R″ is not —CF₃. In yet another embodiment, in Formula IA, each R is independently (i) deuterium, cyano, halo, or nitro; or (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, heterocyclyl, or —OR^{1a}, each of which is optionally substituted with one or more substituents Q; wherein R^{1a} is as defined herein; and R″ is not —CF₃.

In one embodiment, in Formula IA, each R is independently (i) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one, two, or three substituents Q; or (ii) —OR^{1a}; wherein R^{1a} and Q are each as defined herein. In another embodiment, in Formula IA, each R is independently C₁₋₆ alkyl, C₃₋₁₀ cycloalkyl, C₆₋₁₄ aryl, or —OC₁₋₆ alkyl; each of which is optionally substituted with one, two, or three substituents Q as defined herein. In yet another embodiment, in Formula IA, each R is independently C₁₋₆ alkyl or C₃₋₁₀ cycloalkyl; each of which is optionally substituted with one, two, or three substituents Q as defined herein. In yet another embodiment, in Formula IA, each R is independently C₁₋₆ alkyl, optionally substituted with one, two, or three substituents Q as defined herein. In yet another embodiment, in Formula IA, each R is independently C₃₋₁₀ cycloalkyl, optionally substituted with one, two, or three substituents Q as defined herein. In yet another embodiment, in Formula IA, each R is independently C₆₋₁₄ aryl, optionally substituted with one, two, or three substituents Q as defined herein. In still another embodiment, in Formula IA, each R is independently —OC₁₋₆ alkyl, optionally substituted with one, two, or three substituents Q as defined herein.

In one embodiment, in Formula IA, each R is independently deuterium, C₁₋₆ alkyl, C₃₋₁₀ cycloalkyl, C₆₋₁₄ aryl, or —OR^{1a}; R′ and R″′ are each independently hydrogen, deuterium, halo, or C₁₋₆ alkyl; R″ is halo, C₁₋₆ alkyl, heterocyclyl, —C(O)OR^{1a}, —C(O)NR^{1b}R^{1C}, —OR^{1a}, —NR^{1b}R^{1c}, —NR^{1a}C(O)R^{1d}, or —NR^{1a}C(O)OR^{1d}; X is —NH— and Y is —S(O)₂—, or X is —S(O)₂— and Y is —NH—; and n is an integer of 2, 3, or 4; wherein each alkyl, cycloalkyl, aryl, and heterocyclyl is optionally substituted with one, two, or three substituents Q; and each R^{1a}, R^{1b}, R^{1c}, R^{1d}, and Q is as defined herein.

In another embodiment, in Formula IA, each R is independently deuterium, C₁₋₄ alkyl, C₃₋₁₀ cycloalkyl, or —OC₁₋₆ alkyl; R′ and R″′ are each independently hydrogen, deuterium, halo, or C₁₋₄ alkyl; R″ is halo, C₁₋₆ alkyl, heterocyclyl, carboxy, —C(O)C₁₋₆ alkyl, —C(O)N(H)C₁₋₆ alkyl, hydroxyl, —OC₁₋₆ alkyl, amino, —N(H)C₁₋₆ alkyl, —N(C₁₋₆ alkyl)₂, —NHC(O)C₁₋₆ alkyl, or —NHC(O)OC₁₋₆ alkyl; X is —NH— and Y is —S(O)₂—, or X is —S(O)₂— and Y is —NH—; and n is an integer of 2 or 3; wherein each alkyl, cycloalkyl, and heterocyclyl is optionally substituted with one or two substituents Q, each Q independently selected from cyano, C₁₋₆ alkyl, heterocyclyl, and —NR^{b}R^{c}, where R^{b} and R^{c} are each as defined herein.

In yet another embodiment, in Formula IA, each R is independently deuterium, C₁₋₄ alkyl, or C₃₋₁₀ cycloalkyl; R′ and R″′ are each independently hydrogen, deuterium, halo, or C₁₋₄ alkyl; R″ is halo, C₁₋₆ alkyl, heterocyclyl, carboxy, —C(O)C₁₋₆ alkyl, —C(O)N(H)C₁₋₆ alkyl, hydroxyl, —OC₁₋₆ alkyl, amino, —N(H)C₁₋₆ alkyl, —N(C₁₋₆ alkyl)₂, —NHC(O)C₁₋₆ alkyl, or —NHC(O)OC₁₋₆ alkyl; X is —NH— and Y is —S(O)₂—, or X is —S(O)₂— and Y is —NH—; and n is an integer of 2 or 3; wherein each alkyl, cycloalkyl, and heterocyclyl is optionally substituted with one or two substituents, each independently selected from cyano, methyl, trifluoromethyl, morpholinyl, amino, and dimethylamino.

In yet another embodiment, in Formula IA, each R is independently methyl, ethyl, propyl, butyl, cyclopropyl, or trifluoromethylcyclopropyl; R′ and R″′ are each independently hydrogen, deuterium, chloro, bromo, or methyl; R″ is (i) amino, bromo, carboxy, or hydroxyl; or (ii) methyl, propyl, piperidyl, methylcarboxy, butylaminocarbonyl, methoxy, ethoxy, propoxy, butoxy, diethylamino, acetamido, or butoxycarbonylamino, each of which is optionally substituted with one or two substituents, each independently selected from cyano, methyl, morpholinyl, amino, and dimethylamino; X is —NH— and Y is —S(O)₂—, or X is —S(O)₂— and Y is —NH—; and n is an integer of 2 or 3.

In yet another embodiment, in Formula IA, each R is independently methyl, ethyl, propyl, butyl, cyclopropyl, or trifluoromethylcyclopropyl; R′ and R″′ are each independently hydrogen, deuterium, chloro, bromo, or methyl; R″ is bromo, methyl, dimethylaminopropyl, morpholinylpropyl, piperidyl, methylpiperidyl, carboxy, methylcarboxy, butylaminocarbonyl, hydroxyl, methoxy, dimethylaminoethoxy, cyanopropoxy, aminobutoxy, amino, diethylamino, acetamido, or butoxycarbonylamino; X is —NH— and Y is —S(O)₂—, or X is —S(O)₂— and Y is —NH—; and n is an integer of 2 or 3.

In still another embodiment, in Formula IA, each R is independently methyl, ethyl, isopropyl, tert-butyl, or 1-trifluoromethylcyclopropyl; R′ and R″′ are each independently hydrogen, deuterium, chloro, bromo, or methyl; R″ is bromo, methyl, 3-dimethylaminopropyl, 3-morpholin-4-ylpropyl, piperid-4-yl, 1-methylpiperid-4-yl, carboxy, methylcarboxy, butylaminocarbonyl, hydroxyl, methoxy, 2-dimethylaminoethoxy, 3-cyanopropoxy, 4-aminobutoxy, amino, diethylamino, acetamido, or tert-butoxycarbonylamino; X is —NH— and Y is —S(O)₂—, or X is —S(O)₂— and Y is —NH—; and n is an integer of 2 or 3.

In another embodiment, provided herein is a biarylsulfonamide of Formula (IIA):

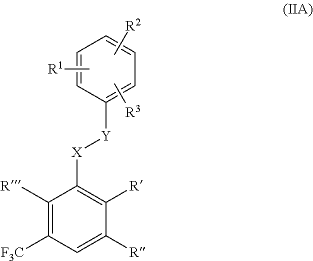

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
R¹ and R³ are each independently C₁₋₆ alkyl, C₂₋₆ alkenyl, C₂₋₆ alkynyl, or C₃₋₁₀ cycloalkyl, each of which is optionally substituted with one or more substituents Q;
R² is (i) hydrogen or deuterium; or (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, heterocyclyl, —OR^{1a}, —C(O)OR^{1a}, —C(O)NR^{1b}R^{c} or —NR^{1b}R^{1c}, each of which is optionally substituted with one or more substituents Q; and
R′, R″, R′″, R^{1a}, R^{1b}, R^{1c}, Q, X, and Y are each as defined herein.

In yet another embodiment, provided herein is a biarylsulfonamide of Formula (IIIA):

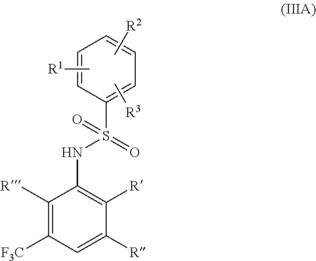

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R′, R″, R′″, R¹, R², and R³ are each as defined herein.

In yet another embodiment, provided herein is a biarylsulfonamide of Formula (IVA):

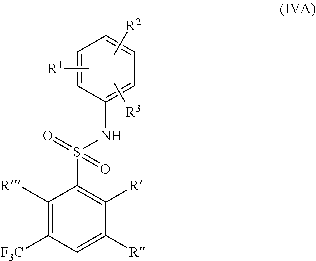

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R′, R″, R′″, R¹, R², and R³ are each as defined herein.

In one embodiment, in Formula IIA, IIIA, or IVA, R² is (i) hydrogen or deuterium; or (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, heterocyclyl, or —OR^{1a}, each of which is optionally substituted with one or more substituents Q; wherein R^{1a} is as defined herein.

In one embodiment, in Formula IIA, IIIA, or IVA, R″ is not —CF₃.

In one embodiment, in Formula IIA, IIIA, or IVA, R¹ and R³ are each independently C₁₋₆ alkyl or C₃₋₁₀ cycloalkyl; R² is hydrogen, deuterium, C₁₋₆ alkyl, C₃₋₁₀ cycloalkyl, C₆₋₁₄ aryl, or —OR^{1a}; R′ and R″′ are each independently hydrogen, deuterium, halo, or C₁₋₆ alkyl; and R″ is halo, C₁₋₆ alkyl, heterocyclyl, —C(O)OR^{1a}, —C(O)NR^{1b}R^{1e}, —OR^{1a}, —NR^{1b}R^{1c}, —NR^{1a}C(O)R^{1d}, or —NR^{1a}C(O)OR^{1d}; wherein each alkyl, cycloalkyl, aryl, and heterocyclyl is optionally substituted with one, two, or three substituents Q; and each R^{1a}, R^{1b}, R^{1c}, R^{1d}, and Q is as defined herein.

In another embodiment, in Formula IIA, IIIA, or IVA, R¹ and R³ are each independently C₁₋₆ alkyl; R² is hydrogen, deuterium, C₁₋₆ alkyl, C₃₋₁₀ cycloalkyl, or —OC₁₋₆ alkyl; R′ and R″′ are each independently hydrogen, deuterium, halo, or C₁₋₄ alkyl; and R″ is halo, C₁₋₆ alkyl, heterocyclyl, carboxy, —C(O)C₁₋₆ alkyl, —C(O)N(H)C₁₋₆ alkyl, hydroxyl, —OC₁₋₆ alkyl, amino, —N(H)C₁₋₆ alkyl, —N(C₁₋₆ alkyl)₂, —NHC(O)C₁₋₆ alkyl, or —NHC(O)OC₁₋₆ alkyl; wherein each alkyl, cycloalkyl, and heterocyclyl is optionally substituted with one or two substituents Q, each Q independently selected from cyano, C₁₋₆ alkyl, heterocyclyl, and —NR^{b}R^{c}, where R^{b} and R^{c} are each as defined herein.

In yet another embodiment, in Formula IIA, IIIA, or IVA, R¹ and R³ are each independently C₁₋₆ alkyl; R² is hydrogen, deuterium, C₁₋₆ alkyl, or C₃₋₁₀ cycloalkyl; R′ and R″′ are each independently hydrogen, deuterium, halo, or C₁₋₄ alkyl; and R″ is halo, C₁₋₆ alkyl, heterocyclyl, carboxy, —C(O)C₁₋₆ alkyl, —C(O)N(H)C₁₋₆ alkyl, hydroxyl, —OC₁₋₆ alkyl, amino, —N(H)C₁₋₆ alkyl, —N(C₁₋₆ alkyl)₂, —NHC(O)C₁₋₆ alkyl, or —NHC(O)OC₁₋₆ alkyl; wherein each alkyl, cycloalkyl, and heterocyclyl is optionally substituted with one or two substituents, each independently selected from cyano, methyl, trifluoromethyl, morpholinyl, amino, and dimethylamino.

In yet another embodiment, in Formula IIA, IIIA, or IVA, R¹ and R³ are each independently methyl, ethyl, or propyl; R² is hydrogen, deuterium, propyl, butyl, cyclopropyl, or trifluoromethylcyclopropyl; R′ and R″′ are each independently hydrogen, deuterium, chloro, bromo, or methyl; and R″ is (i) amino, bromo, carboxy, or hydroxyl; or (ii) methyl, propyl, piperidyl, methylcarboxy, butylaminocarbonyl, methoxy, ethoxy, propoxy, butoxy, diethylamino, acetamido, or butoxycarbonylamino, each of which is optionally substituted with one or two substituents, each independently selected from cyano, methyl, morpholinyl, amino, and dimethylamino.

In yet another embodiment, in Formula IIA, IIIA, or IVA, R¹ and R³ are each independently methyl, ethyl, or propyl; R² is hydrogen, deuterium, propyl, butyl, cyclopropyl, or trifluoromethylcyclopropyl; R′ and R″′ are each independently hydrogen, deuterium, chloro, bromo, or methyl; and R″ is bromo, methyl, dimethylaminopropyl, morpholinylpropyl, piperidyl, methylpiperidyl, carboxy, methylcarboxy, butylaminocarbonyl, hydroxyl, methoxy, dimethylaminoethoxy, cyanopropoxy, aminobutoxy, amino, diethylamino, acetamido, or butoxycarbonylamino.

In still another embodiment, in Formula IIA, IIIA, or IVA, R¹ and R³ are each independently methyl, ethyl, or isopropyl; R² is hydrogen, deuterium, isopropyl, t-butyl, cyclopropyl, or 1-trifluoromethylcyclopropyl; R′ and R″′ are each independently hydrogen, deuterium, chloro, bromo, or methyl; and R″ is bromo, methyl, 3-dimethylaminopropyl, 3-morpholin-4-ylpropyl, piperid-4-yl, 1-methylpiperid-4-yl, carboxy, methylcarboxy, butylaminocarbonyl, hydroxyl, methoxy, 2-dimethylaminoethoxy, 3-cyanopropoxy, 4-aminobutoxy, amino, diethylamino, acetamido, or tert-butoxycarbonylamino.

In yet another embodiment, provided herein is a biarylsulfonamide of Formula (VA):

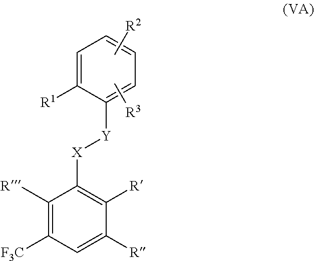

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R′, R″, R′″, R¹, R², R³, X, and Y are each as defined herein.

In yet another embodiment, provided herein is a biarylsulfonamide of Formula (VIA):

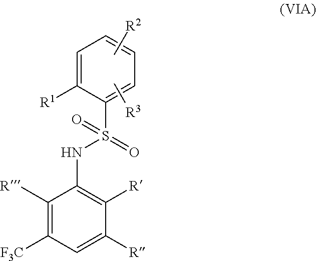

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R′, R″, R′″, R¹, R², and R³ are each as defined herein.

In yet another embodiment, provided herein is a biarylsulfonamide of Formula (VIIA):

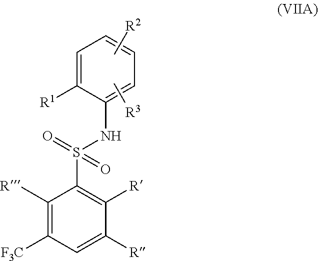

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R′, R″, R′″, R¹, R², and R³ are each as defined herein.

In one embodiment, in Formula VA, VIA, or VIIA, R² is (i) hydrogen or deuterium; or (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, heterocyclyl, or —OR^{1a}, each of which is optionally substituted with one or more substituents Q; wherein R^{1a} is as defined herein.

In one embodiment, in Formula VA, VIA, or VIIA, R″ is not —CF₃.

In one embodiment, in Formula VA, VIA, or VIIA, R¹ and R³ are each independently C₁₋₆ alkyl or C₃₋₁₀ cycloalkyl; R² is hydrogen, deuterium, C₁₋₆ alkyl, C₃₋₁₀ cycloalkyl, C₆₋₁₄ aryl, or —OR^{1a}; R′ and R″′ are each independently hydrogen, deuterium, halo, or C₁₋₆ alkyl; and R″ is halo, C₁₋₆ alkyl, heterocyclyl, —C(O)OR^{1a}, —C(O)NR^{1b}R^{1c}, —OR^{1a}, —NR^{1b}R^{1c}, —NR^{1a}C(O)R^{1d}, or —NR^{1a}C(O)OR^{1d}; wherein each alkyl, cycloalkyl, aryl, and heterocyclyl is optionally substituted with one, two, or three substituents Q; and each R^{1a}, R^{1b}, R^{1c}, R^{1d}, and Q is as defined herein.

In another embodiment, in Formula VA, VIA, or VIIA, R¹ and R³ are each independently C₁₋₆ alkyl; R² is hydrogen, deuterium, C₁₋₆ alkyl, C₃₋₁₀ cycloalkyl, or —OC₁₋₆ alkyl; R′ and R″′ are each independently hydrogen, deuterium, halo, or C₁₋₄ alkyl; and R″ is halo, C₁₋₆ alkyl, heterocyclyl, carboxy, —C(O)C₁₋₆ alkyl, —C(O)N(H)C₁₋₆ alkyl, hydroxyl, —OC₁₋₆ alkyl, amino, —N(H)C₁₋₆ alkyl, —N(C₁₋₆ alkyl)₂, —NHC(O)C₁₋₆ alkyl, or —NHC(O)OC₁₋₆ alkyl; wherein each alkyl, cycloalkyl, and heterocyclyl is optionally substituted with one or two substituents Q, each Q independently selected from cyano, C₁₋₆ alkyl, heterocyclyl, and —NR^{b}R^{c}, where R^{b} and R^{c} are each as defined herein.

In yet another embodiment, in Formula VA, VIA, or VIIA, R¹ and R³ are each independently C₁₋₆ alkyl; R² is hydrogen, deuterium, C₁₋₆ alkyl, or C₃₋₁₀ cycloalkyl; R′ and R″′ are each independently hydrogen, deuterium, halo, or C₁₋₄ alkyl; and R″ is halo, C₁₋₆ alkyl, heterocyclyl, carboxy, —C(O)C₁₋₆ alkyl, —C(O)N(H)C₁₋₆ alkyl, hydroxyl, —OC₁₋₆ alkyl, amino, —N(H)C₁₋₆ alkyl, —N(C₁₋₆ alkyl)₂, —NHC(O)C₁₋₆ alkyl, or —NHC(O)OC₁₋₆ alkyl; wherein each alkyl, cycloalkyl, and heterocyclyl is optionally substituted with one or two substituents, each independently selected from cyano, methyl, trifluoromethyl, morpholinyl, amino, and dimethylamino.

In yet another embodiment, in Formula VA, VIA, or VIIA, R¹ and R³ are each independently methyl, ethyl, or propyl; R² is hydrogen, deuterium, propyl, butyl, cyclopropyl, or trifluoromethylcyclopropyl; R′ and R″′ are each independently hydrogen, deuterium, chloro, bromo, or methyl; and R″ is (i) amino, bromo, carboxy, or hydroxyl; or (ii) methyl, propyl, piperidyl, methylcarboxy, butylaminocarbonyl, methoxy, ethoxy, propoxy, butoxy, diethylamino, acetamido, or butoxycarbonylamino, each of which is optionally substituted with one or two substituents, each independently selected from cyano, methyl, morpholinyl, amino, and dimethylamino.

In yet another embodiment, in Formula VA, VIA, or VIIA, R¹ and R³ are each independently methyl, ethyl, or propyl; R² is hydrogen, deuterium, propyl, butyl, cyclopropyl, or trifluoromethylcyclopropyl; R′ and R″′ are each independently hydrogen, deuterium, chloro, bromo, or methyl; and R″ is bromo, methyl, dimethylaminopropyl, morpholinylpropyl, piperidyl, methylpiperidyl, carboxy, methylcarboxy, butylaminocarbonyl, hydroxyl, methoxy, dimethylaminoethoxy, cyanopropoxy, aminobutoxy, amino, diethylamino, acetamido, or butoxycarbonylamino.

In still another embodiment, in Formula VA, VIA, or VIIA, R¹ and R³ are each independently methyl, ethyl, or isopropyl; R² is hydrogen, deuterium, isopropyl, t-butyl, cyclopropyl, or 1-trifluoromethylcyclopropyl; R′ and R″′ are each independently hydrogen, deuterium, chloro, bromo, or methyl; and R″ is bromo, methyl, 3-dimethylaminopropyl, 3-morpholin-4-ylpropyl, piperid-4-yl, 1-methylpiperid-4-yl, carboxy, methylcarboxy, butylaminocarbonyl, hydroxyl, methoxy, 2-dimethylaminoethoxy, 3-cyanopropoxy, 4-aminobutoxy, amino, diethylamino, acetamido, or tert-butoxycarbonylamino.

In yet another embodiment, provided herein is a biarylsulfonamide of Formula (VIIIA):

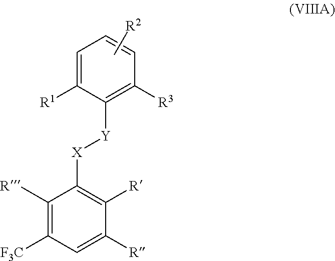

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R′, R″, R′″, R¹, R², R³, X, and Y are each as defined herein.

In yet another embodiment, provided herein is a biarylsulfonamide of Formula (IXA):

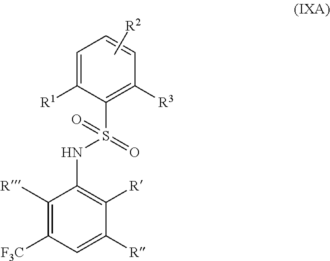

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R′, R″, R′″, R¹, R², and R³ are each as defined herein.

In yet another embodiment, provided herein is a biarylsulfonamide of Formula (XA):

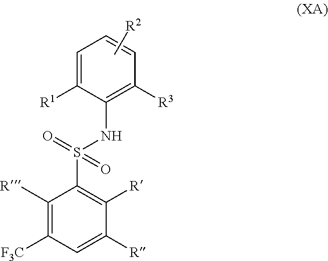

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R′, R″, R′″, R¹, R², and R³ are each as defined herein.

In one embodiment, in Formula VIIIA, IXA, or XA, R² is (i) hydrogen or deuterium; or (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, heterocyclyl, or —OR^{1a}, each of which is optionally substituted with one or more substituents Q; wherein R^{1a} is as defined herein.

In one embodiment, in Formula VIIIA, IXA, or XA, R″ is not —CF₃.

In one embodiment, in Formula VIIIA, IXA, or XA, R¹ and R³ are each independently C₁₋₆ alkyl or C₃₋₁₀ cycloalkyl; R² is hydrogen, deuterium, C₁₋₆ alkyl, C₃₋₁₀ cycloalkyl, C₆₋₁₄ aryl, or —OR^{1a}; R′ and R″′ are each independently hydrogen, deuterium, halo, or C₁₋₆ alkyl; and R″ is halo, C₁₋₆ alkyl, heterocyclyl, —C(O)OR^{1a}, —C(O)NR^{1b}R^{1c}, —OR^{1a}, —NR^{1b}R^{1c}, —NR^{1a}C(O)R^{1d}, or —NR^{1a}C(O)OR^{1d}; wherein each alkyl, cycloalkyl, aryl, and heterocyclyl is optionally substituted with one, two, or three substituents Q; and each R^{1a}, R^{1b}, R^{1c}, R^{1d}, and Q is as defined herein.

In another embodiment, in Formula VIIIA, IXA, or XA, R¹ and R³ are each independently C₁₋₆ alkyl; R² is hydrogen, deuterium, C₁₋₆ alkyl, C₃₋₁₀ cycloalkyl, or —OC₁₋₆ alkyl; R′ and R″′ are each independently hydrogen, deuterium, halo, or C₁₋₄ alkyl; and R″ is halo, C₁₋₆ alkyl, heterocyclyl, carboxy, —C(O)C₁₋₆ alkyl, —C(O)N(H)C₁₋₆ alkyl, hydroxyl, —OC₁₋₆ alkyl, amino, —N(H)C₁₋₆ alkyl, —N(C₁₋₆ alkyl)₂, —NHC(O)C₁₋₆ alkyl, or —NHC(O)OC₁₋₆ alkyl; wherein each alkyl, cycloalkyl, and heterocyclyl is optionally substituted with one or two substituents Q, each Q independently selected from cyano, C₁₋₆ alkyl, heterocyclyl, and —NR^{b}R^{c}, where R^{b} and R^{c} are each as defined herein.

In yet another embodiment, in Formula VIIIA, IXA, or XA, R¹ and R³ are each independently C₁₋₆ alkyl; R² is hydrogen, deuterium, C₁₋₆ alkyl, or C₃₋₁₀ cycloalkyl; R′ and R″′ are each independently hydrogen, deuterium, halo, or C₁₋₄ alkyl; and R″ is halo, C₁₋₆ alkyl, heterocyclyl, carboxy, —C(O)C₁₋₆ alkyl, —C(O)N(H)C₁₋₆ alkyl, hydroxyl, —OC₁₋₆ alkyl, amino, —N(H)C₁₋₆ alkyl, —N(C₁₋₆ alkyl)₂, —NHC(O)C₁₋₆ alkyl, or —NHC(O)OC₁₋₆ alkyl; wherein each alkyl, cycloalkyl, and heterocyclyl is optionally substituted with one or two substituents, each independently selected from cyano, methyl, trifluoromethyl, morpholinyl, amino, and dimethylamino.

In yet another embodiment, in Formula VIIIA, IXA, or XA, R¹ and R³ are each independently methyl, ethyl, or propyl; R² is hydrogen, deuterium, propyl, butyl, cyclopropyl, or trifluoromethylcyclopropyl; R′ and R″′ are each independently hydrogen, deuterium, chloro, bromo, or methyl; and R″ is (i) amino, bromo, carboxy, or hydroxyl; or (ii) methyl, propyl, piperidyl, methylcarboxy, butylaminocarbonyl, methoxy, ethoxy, propoxy, butoxy, diethylamino, acetamido, or butoxycarbonylamino, each of which is optionally substituted with one or two substituents, each independently selected from cyano, methyl, morpholinyl, amino, and dimethylamino.

In yet another embodiment, in Formula VIIIA, IXA, or XA, R¹ and R³ are each independently methyl, ethyl, or propyl; R² is hydrogen, deuterium, propyl, butyl, cyclopropyl, or trifluoromethylcyclopropyl; R′ and R″′ are each independently hydrogen, deuterium, chloro, bromo, or methyl; and R″ is bromo, methyl, dimethylaminopropyl, morpholinylpropyl, piperidyl, methylpiperidyl, carboxy, methylcarboxy, butylaminocarbonyl, hydroxyl, methoxy, dimethylaminoethoxy, cyanopropoxy, aminobutoxy, amino, diethylamino, acetamido, or butoxycarbonylamino.

In still another embodiment, in Formula VIIIA, IXA, or XA, R¹ and R³ are each independently methyl, ethyl, or isopropyl; R² is hydrogen, deuterium, isopropyl, t-butyl, cyclopropyl, or 1-trifluoromethylcyclopropyl; R′ and R″′ are each independently hydrogen, deuterium, chloro, bromo, or methyl; and R″ is bromo, methyl, 3-dimethylaminopropyl, 3-morpholin-4-ylpropyl, piperid-4-yl, 1-methylpiperid-4-yl, carboxy, methylcarboxy, butylaminocarbonyl, hydroxyl, methoxy, 2-dimethylaminoethoxy, 3-cyanopropoxy, 4-aminobutoxy, amino, diethylamino, acetamido, or tert-butoxycarbonylamino.

In yet another embodiment, provided herein is a biarylsulfonamide of Formula (XIA):

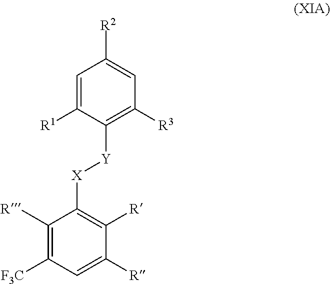

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R′, R″, R′″, R¹, R², R³, X, and Y are each as defined herein.

In yet another embodiment, provided herein is a biarylsulfonamide of Formula (XIIA):

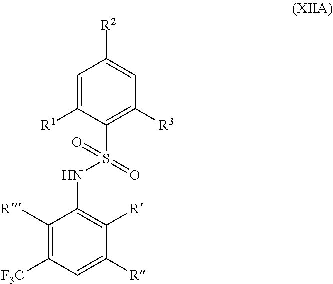

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R′, R″, R′″, R¹, R², and R³ are each as defined herein.

In still another embodiment, provided herein is a biarylsulfonamide of Formula (XIII):

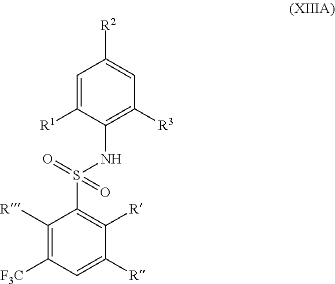

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R′, R″, R′″, R¹, R², and R³ are each as defined herein.

In one embodiment, in Formula XIA, XIIA, or XIIIA, R² is (i) hydrogen or deuterium; or (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, heterocyclyl, or —OR^{1a}, each of which is optionally substituted with one or more substituents Q; wherein R^{1a} is as defined herein.

In one embodiment, in Formula XIA, XIIA, or XIIIA, R″ is not —CF₃.

In one embodiment, in Formula XIA, XIIA, or XIIIA, R¹ and R³ are each independently C₁₋₆ alkyl or C₃₋₁₀ cycloalkyl; R² is hydrogen, deuterium, C₁₋₆ alkyl, C₃₋₁₀ cycloalkyl, C₆₋₁₄ aryl, or —OR^{1a}; R′ and R″′ are each independently hydrogen, deuterium, halo, or C₁₋₆ alkyl; and R″ is halo, C₁₋₆ alkyl, heterocyclyl, —C(O)OR^{1a}, —C(O)NR^{1b}R^{1c}, —OR^{1a}, —NR^{1b}R^{1c}, —NR^{1a}C(O)R^{1d}, or —NR^{1a}C(O)OR^{1d}; wherein each alkyl, cycloalkyl, aryl, and heterocyclyl is optionally substituted with one, two, or three substituents Q; and each R^{1a}, R^{1b}, R^{1c}, R^{1d}, and Q is as defined herein.

In another embodiment, in Formula XIA, XIIA, or XIIIA, R¹ and R³ are each independently C₁₋₆ alkyl; R² is hydrogen, deuterium, C₁₋₆ alkyl, C₃₋₁₀ cycloalkyl, or —OC₁₋₆ alkyl; R′ and R″′ are each independently hydrogen, deuterium, halo, or C₁₋₄ alkyl; and R″ is halo, C₁₋₆ alkyl, heterocyclyl, carboxy, —C(O)C₁₋₆ alkyl, —C(O)N(H)C₁₋₆ alkyl, hydroxyl, —OC₁₋₆ alkyl, amino, —N(H)C₁₋₆ alkyl, —N(C₁₋₆ alkyl)₂, —NHC(O)C₁₋₆ alkyl, or —NHC(O)OC₁₋₆ alkyl; wherein each alkyl, cycloalkyl, and heterocyclyl is optionally substituted with one or two substituents Q, each Q independently selected from cyano, C₁₋₆ alkyl, heterocyclyl, and —NR^{b}R^{c}, where R^{b} and R^{c} are each as defined herein.

In yet another embodiment, in Formula XIA, XIIA, or XIIIA, R¹ and R³ are each independently C₁₋₆ alkyl; R² is hydrogen, deuterium, C₁₋₆ alkyl, or C₃₋₁₀ cycloalkyl; R′ and R″′ are each independently hydrogen, deuterium, halo, or C₁₋₄ alkyl; and R″ is halo, C₁₋₆ alkyl, heterocyclyl, carboxy, —C(O)C₁₋₆ alkyl, —C(O)N(H)C₁₋₆ alkyl, hydroxyl, —OC₁₋₆ alkyl, amino, —N(H)C₁₋₆ alkyl, —N(C₁₋₆ alkyl)₂, —NHC(O)C₁₋₆ alkyl, or —NHC(O)OC₁₋₆ alkyl; wherein each alkyl, cycloalkyl, and heterocyclyl is optionally substituted with one or two substituents, each independently selected from cyano, methyl, trifluoromethyl, morpholinyl, amino, and dimethylamino.

In yet another embodiment, in Formula XIA, XIIA, or XIIIA, R¹ and R³ are each independently methyl, ethyl, or propyl; R² is hydrogen, deuterium, propyl, butyl, cyclopropyl, or trifluoromethylcyclopropyl; R′ and R″′ are each independently hydrogen, deuterium, chloro, bromo, or methyl; and R″ is (i) amino, bromo, carboxy, or hydroxyl; or (ii) methyl, propyl, piperidyl, methylcarboxy, butylaminocarbonyl, methoxy, ethoxy, propoxy, butoxy, diethylamino, acetamido, or butoxycarbonylamino, each of which is optionally substituted with one or two substituents, each independently selected from cyano, methyl, morpholinyl, amino, and dimethylamino.

In yet another embodiment, in Formula XIA, XIIA, or XIIIA, R¹ and R³ are each independently methyl, ethyl, or propyl; R² is hydrogen, deuterium, propyl, butyl, cyclopropyl, or trifluoromethylcyclopropyl; R′ and R″′ are each independently hydrogen, deuterium, chloro, bromo, or methyl; and R″ is bromo, methyl, dimethylaminopropyl, morpholinylpropyl, piperidyl, methylpiperidyl, carboxy, methylcarboxy, butylaminocarbonyl, hydroxyl, methoxy, dimethylaminoethoxy, cyanopropoxy, aminobutoxy, amino, diethylamino, acetamido, or butoxycarbonylamino.

In still another embodiment, in Formula XIA, XIIA, or XIIIA, R¹ and R³ are each independently methyl, ethyl, or isopropyl; R² is hydrogen, deuterium, isopropyl, t-butyl, cyclopropyl, or 1-trifluoromethylcyclopropyl; R′ and R″′ are each independently hydrogen, deuterium, chloro, bromo, or methyl; and R″ is bromo, methyl, 3-dimethylaminopropyl, 3-morpholin-4-ylpropyl, piperid-4-yl, 1-methylpiperid-4-yl, carboxy, methylcarboxy, butylaminocarbonyl, hydroxyl, methoxy, 2-dimethylaminoethoxy, 3-cyanopropoxy, 4-aminobutoxy, amino, diethylamino, acetamido, or tert-butoxycarbonylamino.

In one embodiment, in any one of Formulae IIA to XIIIA, R² is (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one, two, or three substituents Q; or (iii) —OR^{1a}; wherein R^{1a} and Q are each as defined herein. In another embodiment, in any one of Formulae IIA to XIIIA, R² is (i) hydrogen or deuterium; or (ii) C₁₋₆ alkyl, C₃₋₁₀ cycloalkyl, C₆₋₁₄ aryl, or —OC₁₋₆ alkyl, each of which is optionally substituted with one, two, or three substituents Q as defined herein. In yet another embodiment, in any one of Formulae IIA to XIIIA, R² is (i) hydrogen or deuterium; or (ii) C₁₋₆ alkyl or C₃₋₁₀ cycloalkyl, each of which is optionally substituted with one, two, or three substituents Q as defined herein. In yet another embodiment, in any one of Formulae IIA to XIIIA, R² is hydrogen or deuterium. In yet another embodiment, in any one of Formulae IIA to XIIIA, R² is C₁₋₆ alkyl, optionally substituted with one, two, or three substituents Q as defined herein. In yet another embodiment, in any one of Formulae IIA to XIIIA, R² is C₃₋₁₀ cycloalkyl, optionally substituted with one, two, or three substituents Q as defined herein. In yet another embodiment, in any one of Formulae IIA to XIIIA, R² is C₆₋₁₄ aryl, optionally substituted with one, two, or three substituents Q as defined herein. In still another embodiment, in any one of Formulae IIA to XIIIA, R² is —OC₁₋₆ alkyl, optionally substituted with one, two, or three substituents Q as defined herein.

In one embodiment, the biarylsulfonamide described herein is:
2,4,6-triisopropyl-N-(3-methyl-5-(trifluoromethyl)phenyl)benzenesulfonamide A1;
methyl 3-(trifluoromethyl)-5-((2,4,6-triisopropylphenyl)sulfonamido)benzoate A2;
3-(trifluoromethyl)-5-((2,4,6-triisopropylphenyl)sulfonamido)benzoic acid A3; N-butyl-3-(trifluoromethyl)-5-((2,4,6-triisopropylphenyl)sulfonamido)benzamide A4;
tert-butyl (3-(trifluoromethyl)-5-((2,4,6-triisopropylphenyl)sulfonamido)-phenyl)carbamate A5;
N-(3-amino-5-(trifluoromethyl)phenyl)-2,4,6-triisopropylbenzenesulfonamide A6;
N-(3-(trifluoromethyl)-5-((2,4,6-triisopropylphenyl)sulfonamido)phenyl)-acetamide A7;
N-(3-(diethylamino)-5-(trifluoromethyl)phenyl)-2,4,6-triisopropylbenzenesulfonamide A8;
4-(tert-butyl)-N-(3-(3-cyanopropoxy)-5-(trifluoromethyl)phenyl)-2,6-dimethylbenzenesulfonamide A9;
N-(3-(4-aminobutoxy)-5-(trifluoromethyl)phenyl)-4-(tert-butyl)-2,6-dimethylbenzenesulfonamide A10;
N-(3-(4-aminobutoxy)-5-(trifluoromethyl)phenyl)-2,4,6-triisopropylbenzenesulfonamide A11;
4-(tert-butyl)-N-(3-(2-(dimethylamino)ethoxy)-5-(trifluoromethyl)phenyl)-2,6-dimethylbenzenesulfonamide A12;
4-(tert-butyl)-N-(3-(3-(dimethylamino)propyl)-5-(trifluoromethyl)phenyl)-2,6-dimethylbenzenesulfonamide A13;
4-(tert-butyl)-2,6-dimethyl-N-(3-(3-morpholinopropyl)-5-(trifluoromethyl)-phenyl)benzenesulfonamide A14;
4-(tert-butyl)-2,6-dimethyl-N-(3-(1-methylpiperidin-4-yl)-5-(trifluoromethyl)-phenyl)benzenesulfonamide A15;
N-(3-bromo-5-(trifluoromethyl)phenyl)-4-(tert-butyl)-2,6-dimethyl-benzene-sulfonamide A16;
4-(tert-butyl)-N-(3-methoxy-5-(trifluoromethyl)phenyl)-2,6-dimethylbenzenesulfonamide A17;
4-(tert-butyl)-N-(3-hydroxy-5-(trifluoromethyl)phenyl)-2,6-dimethylbenzene-sulfonamide A18;
2,4,6-triisopropyl-N-(5-methoxy-2-methyl-3-(trifluoromethyl)phenyl)-benzenesulfonamide A19;
N-(5-hydroxy-2-methyl-3-(trifluoromethyl)phenyl)-2,4,6-triisopropylbenzenesulfonamide A20;
N-(3-bromo-2-methyl-5-(trifluoromethyl)phenyl)-2,4,6-triisopropylbenzenesulfonamide A21;
4-(tert-butyl)-2,6-dimethyl-N-(3-(piperidin-4-yl)-5-(trifluoromethyl)phenyl)-benzenesulfonamide A22; or
N-(3-(3-cyanopropoxy)-5-(trifluoromethyl)phenyl)-2,4,6-triisopropylbenzenesulfonamide A23;
   or a tautomer, a mixture of two or more tautomers, or an isotopic variant thereof; or a pharmaceutically acceptable salt, solvate, hydrate, or prodrug thereof.

In one embodiment, provided herein is a biarylsulfonamide of Formula (I):

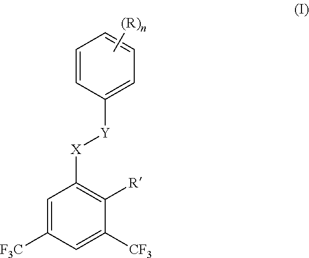

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
each R is independently deuterium, cyano, halo, nitro, C₁₋₆ alkyl, C₂₋₆ alkenyl, C₂₋₆ alkynyl, or C₃₋₁₀ cycloalkyl;
R′ is (i) hydrogen, deuterium, cyano, halo, or nitro; (ii) C₁₋₆ alkyl, C₂₋₆ alkenyl, C₂-6 alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c} or —S(O)₂NR^{1b}R^{1c};
each R^{1a}, R^{1b}, R^{1c}, and R^{1d} is independently hydrogen, deuterium, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or R^{1a} and R^{1c} together with the C and N atoms to which they are attached form heterocyclyl; or R^{1b} and R^{1c} together with the N atom to which they are attached form heterocyclyl;
X is —NH— and Y is —S(O)₂—; or X is —S(O)₂— and Y is —NH—; and
n is an integer of 2, 3, or 4;
wherein each alkyl, alkenyl, alkynyl, cycloalkyl, aryl, aralkyl, heteroaryl, and heterocyclyl is optionally substituted with one or more, in one embodiment, one, two, three, or four, substituents Q, wherein each Q is independently: (a) deuterium, cyano, halo, nitro, or oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is further optionally substituted with one or more, in one embodiment, one, two, three, or four, substituents Q^{a}; or (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, or —S(O)₂NR^{b}R^{c}, wherein each R^{a}, R^{b}, R′, and R^{d} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl optionally substituted with one or more, in one embodiment, one, two, three, or four, substituents Q^{a};
wherein each Q^{a} is independently: (a) deuterium, cyano, halo, nitro, or oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; and (c) —C(O)R^{e}, —C(O)OR^{e}, —C(O)NR^{f}R^{g}, —C(O)SR^{e}, —C(NR^{c})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{c})NR^{f}R^{g}, —OC(S)R^{e}, —OC(S)OR^{e}, —OC(S)NR^{f}R^{g}, —OS(O)R^{e}, —OS(O)₂R^{1c}, —OS(O)NR^{f}R^{g}, —OS(O)₂NR^{f}R^{g}, —NR^{f}R^{g}, —NR^{c}C(O)R^{h}, —NR^{c}C(O)OR^{f}, —NR^{c}C(O)NR^{f}R^{g}, —NR^{e}C(O)SR^{f}, —NR^{c}C(NR^{h})NR^{f}R^{g}, —NR^{e}C(S)R^{h}, —NR^{e}C(S)OR^{f}, —NR^{c}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{c}, —S(O)R^{1c}, —S(O)₂R^{1c}, —S(O)NR^{f}R^{g}, or —S(O)₂NR^{f}R^{g}; wherein each R^{c}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In one embodiment, in Formula I, each R is independently deuterium, C₁₋₆ alkyl, or C₃₋₁₀ cycloalkyl; R′ is hydrogen, deuterium, halo, or C₁₋₆ alkyl; X is —NH— and Y is —S(O)₂—, or X is —S(O)₂— and Y is —NH—; and n is an integer of 2, 3, or 4; wherein each alkyl and cycloalkyl is optionally substituted with one or more substituents Q as defined herein.

In another embodiment, in Formula I, each R is independently deuterium or C₁₋₆ alkyl; R′ is hydrogen, deuterium, halo, or C₁₋₆ alkyl; X is —NH— and Y is —S(O)₂—, or X is —S(O)₂— and Y is —NH—; and n is an integer of 2 or 3; wherein each alkyl is optionally substituted with one or more substituents Q as defined herein.

In yet another embodiment, in Formula I, each R is independently deuterium or C₁₋₄ alkyl; R′ is hydrogen, deuterium, halo, or C₁₋₄ alkyl; X is —NH— and Y is —S(O)₂—, or X is —S(O)₂— and Y is —NH—; and n is an integer of 2 or 3.

In yet another embodiment, in Formula I, each R is independently methyl, ethyl, propyl, or butyl; R′ is hydrogen, deuterium, chloro, bromo, or methyl; X is —NH— and Y is —S(O)₂—, or X is —S(O)₂— and Y is —NH—; and n is an integer of 2 or 3.

In still another embodiment, in Formula I, each R is independently methyl, ethyl, isopropyl, or t-butyl; R′ is hydrogen, deuterium, chloro, bromo, or methyl; X is —NH— and Y is —S(O)₂—, or X is —S(O)₂— and Y is —NH—; and n is an integer of 2 or 3.

In another embodiment, provided herein is a biarylsulfonamide of Formula (II):

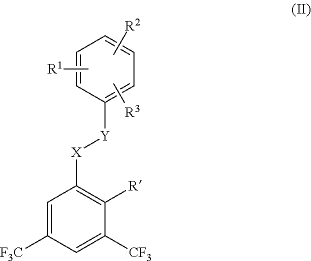

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
R¹ and R³ are each independently C₁₋₆ alkyl, C₂₋₆ alkenyl, C₂₋₆ alkynyl, or C₃₋₁₀ cycloalkyl, each of which is optionally substituted with one or more substituents Q;
R² is (i) hydrogen or deuterium; or (ii) C₁₋₆ alkyl, C₂₋₆ alkenyl, C₂₋₆ alkynyl, or C₃₋₁₀ cycloalkyl, each of which is optionally substituted with one or more substituents Q; and
R′, Q, X, and Y are each as defined herein.

In yet another embodiment, provided herein is a biarylsulfonamide of Formula (III):

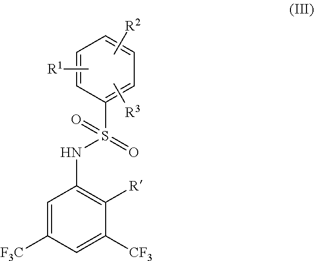

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R′, R¹, R², and R³ are each as defined herein.

In yet another embodiment, provided herein is a biarylsulfonamide of Formula (IV):

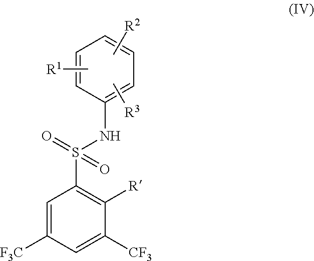

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R′, R¹, R², and R³ are each as defined herein.

In one embodiment, in Formula II, III, or IV, R¹ and R³ are each independently C₁₋₆ alkyl or C₃₋₁₀ cycloalkyl; R² is hydrogen, deuterium, C₁₋₆ alkyl, or C₃₋₁₀ cycloalkyl; and R′ is hydrogen, deuterium, halo, or C₁₋₆ alkyl; wherein each alkyl and cycloalkyl is optionally substituted with one or more substituents Q as defined herein.

In another embodiment, in Formula II, III, or IV, R¹ and R³ are each independently C₁₋₆ alkyl; R² is hydrogen, deuterium, or C₁₋₆ alkyl; and R′ is hydrogen, deuterium, halo, or C₁₋₆ alkyl; wherein each alkyl is optionally substituted with one or more substituents Q as defined herein.

In yet another embodiment, in Formula II, III, or IV, R¹ and R³ are each independently C₁₋₄ alkyl; R² is hydrogen, deuterium, or C₁₋₄ alkyl; and R′ is hydrogen, deuterium, halo, or C₁₋₄ alkyl.

In yet another embodiment, in Formula II, III, or IV, R¹ and R³ are each independently methyl, ethyl, or propyl; R² is hydrogen, deuterium, propyl, or butyl; and R′ is hydrogen, deuterium, chloro, bromo, or methyl.

In still another embodiment, in Formula II, III, or IV, R¹ and R³ are each independently methyl, ethyl, or isopropyl; R² is hydrogen, deuterium, isopropyl, or t-butyl; and R′ is hydrogen, deuterium, chloro, bromo, or methyl.

In yet another embodiment, provided herein is a biarylsulfonamide of Formula (V):

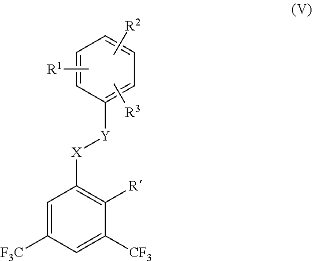

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R′, R¹, R², R³, X, and Y are each as defined herein.

In yet another embodiment, provided herein is a biarylsulfonamide of Formula (VI):

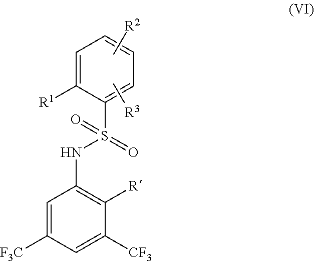

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R′, R¹, R², and R³ are each as defined herein.

In yet another embodiment, provided herein is a biarylsulfonamide of Formula (VII):

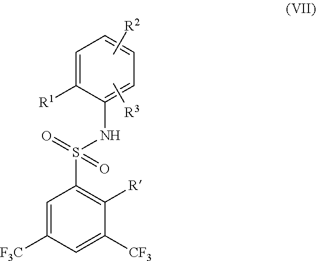

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R′, R¹, R², and R³ are each as defined herein.

In one embodiment, in Formula V, VI, or VII, R¹ and R³ are each independently C₁₋₆ alkyl or C₃₋₁₀ cycloalkyl; R² is hydrogen, deuterium, C₁₋₆ alkyl, or C₃₋₁₀ cycloalkyl; and R′ is hydrogen, deuterium, halo, or C₁₋₆ alkyl; wherein each alkyl and cycloalkyl is optionally substituted with one or more substituents Q as defined herein.

In another embodiment, in Formula V, VI, or VII, R¹ and R³ are each independently C₁₋₆ alkyl; R² is hydrogen, deuterium, or C₁₋₆ alkyl; and R′ is hydrogen, deuterium, halo, or C₁₋₆ alkyl; wherein each alkyl is optionally substituted with one or more substituents Q as defined herein.

In yet another embodiment, in Formula V, VI, or VII, R¹ and R³ are each independently C₁₋₄ alkyl; R² is hydrogen, deuterium, or C₁₋₄ alkyl; and R′ is hydrogen, deuterium, halo, or C₁₋₄ alkyl.

In yet another embodiment, in Formula V, VI, or VII, R¹ and R³ are each independently methyl, ethyl, or propyl; R² is hydrogen, deuterium, propyl, or butyl; and R′ is hydrogen, deuterium, chloro, bromo, or methyl.

In still another embodiment, in Formula V, VI, or VII, R¹ and R³ are each independently methyl, ethyl, or isopropyl; R² is hydrogen, deuterium, isopropyl, or t-butyl; and R′ is hydrogen, deuterium, chloro, bromo, or methyl.

In yet another embodiment, provided herein is a biarylsulfonamide of Formula (VIII):

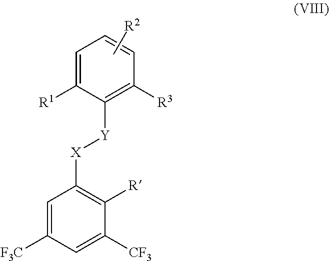

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R′, R¹, R², R³, X, and Y are each as defined herein.

In yet another embodiment, provided herein is a biarylsulfonamide of Formula (IX):

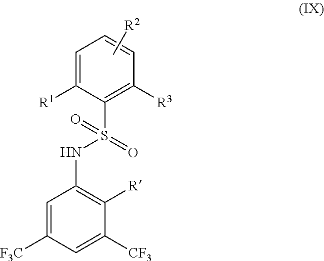

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R′, R¹, R², and R³ are each as defined herein.

In yet another embodiment, provided herein is a biarylsulfonamide of Formula (X):

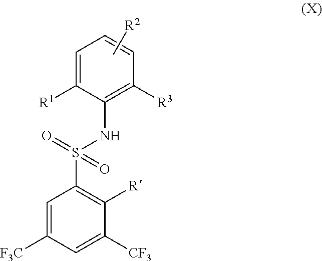

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R′, R¹, R², and R³ are each as defined herein.

In one embodiment, in Formula VIII, IX, or X, R¹ and R³ are each independently C₁₋₆ alkyl or C₃₋₁₀ cycloalkyl; R² is hydrogen, deuterium, C₁₋₆ alkyl, or C₃₋₁₀ cycloalkyl; and R′ is hydrogen, deuterium, halo, or C₁₋₆ alkyl; wherein each alkyl and cycloalkyl is optionally substituted with one or more substituents Q as defined herein.

In another embodiment, in Formula VIII, IX, or X, R¹ and R³ are each independently C₁₋₆ alkyl; R² is hydrogen, deuterium, or C₁₋₆ alkyl; and R′ is hydrogen, deuterium, halo, or C₁₋₆ alkyl; wherein each alkyl is optionally substituted with one or more substituents Q as defined herein.

In yet another embodiment, in Formula VIII, IX, or X, R¹ and R³ are each independently C₁₋₄ alkyl; R² is hydrogen, deuterium, or C₁₋₄ alkyl; and R′ is hydrogen, deuterium, halo, or C₁₋₄ alkyl.

In yet another embodiment, in Formula VIII, IX, or X, R¹ and R³ are each independently methyl, ethyl, or propyl; R² is hydrogen, deuterium, propyl, or butyl; and R′ is hydrogen, deuterium, chloro, bromo, or methyl.

In still another embodiment, in Formula VIII, IX, or X, R¹ and R³ are each independently methyl, ethyl, or isopropyl; R² is hydrogen, deuterium, isopropyl, or t-butyl; and R′ is hydrogen, deuterium, chloro, bromo, or methyl.

In yet another embodiment, provided herein is a biarylsulfonamide of Formula (XI):

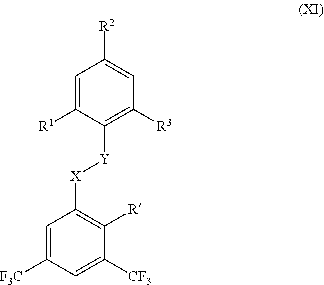

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R′, R¹, R², R³, X, and Y are each as defined herein.

In yet another embodiment, provided herein is a biarylsulfonamide of Formula (XII):

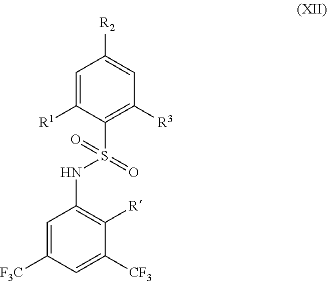

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R′, R¹, R², and R³ are each as defined herein.

In still another embodiment, provided herein is a biarylsulfonamide of Formula (XIII):

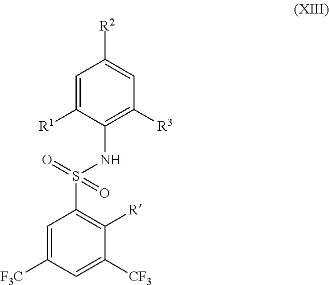

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R′. R¹, R², and R³ are each as defined herein.

In one embodiment, in Formula XI, XII, or XIII, R¹ and R³ are each independently C₁₋₆ alkyl or C₃₋₁₀ cycloalkyl; R² is hydrogen, deuterium, C₁₋₆ alkyl, or C₃₋₁₀ cycloalkyl; and R′ is hydrogen, deuterium, halo, or C₁₋₆ alkyl; wherein each alkyl and cycloalkyl is optionally substituted with one or more substituents Q as defined herein.

In another embodiment, in Formula XI, XII, or XIII, R¹ and R³ are each independently C₁₋₆ alkyl; R² is hydrogen, deuterium, or C₁₋₆ alkyl; and R′ is hydrogen, deuterium, halo, or C₁₋₆ alkyl; wherein each alkyl is optionally substituted with one or more substituents Q as defined herein.

In yet another embodiment, in Formula XI, XII, or XIII, R¹ and R³ are each independently C₁₋₄ alkyl; R² is hydrogen, deuterium, or C₁₋₄ alkyl; and R′ is hydrogen, deuterium, halo, or C₁₋₄ alkyl.

In yet another embodiment, in Formula XI, XII, or XIII, R¹ and R³ are each independently methyl, ethyl, or propyl; R² is hydrogen, deuterium, propyl, or butyl; and R′ is hydrogen, deuterium, chloro, bromo, or methyl.

In still another embodiment, in Formula XI, XII, or XIII, R¹ and R³ are each independently methyl, ethyl, or isopropyl; R² is hydrogen, deuterium, isopropyl, or t-butyl; and R′ is hydrogen, deuterium, chloro, bromo, or methyl.

In one embodiment, the biarylsulfonamide provided herein is:
N-(3,5-bis(trifluoromethyl)phenyl)-2,4,6-triisopropylbenzenesulfonamide B1;
N-(2-chloro-3,5-bis(trifluoromethyl)phenyl)-2,4,6-triisopropyl-benzene-sulfonamide B2;
N-(2-bromo-3,5-bis(trifluoromethyl)phenyl)-2,4,6-triisopropyl-benzene-sulfonamide B3;
N-(2-methyl-3,5-bis(trifluoromethyl)phenyl)-2,4,6-triisopropyl-benzene-sulfonamide B4;
N-(3,5-bis(trifluoromethyl)phenyl)-4-(tert-butyl)-2,6-dimethylbenzene-sulfonamide B5;
N-(2-chloro-3,5-bis(trifluoromethyl)phenyl)-4-(tert-butyl)-2,6-dimethylbenzene-sulfonamide B6;
N-(2-methyl-3,5-bis(trifluoromethyl)phenyl)-4-(tert-butyl)-2,6-dimethylbenzene-sulfonamide B7;
N-(3,5-bis(trifluoromethyl)phenyl)-2,6-diethylbenzenesulfonamide B8;
N-(2-methyl-3,5-bis(trifluoromethyl)phenyl)-2,6-diethylbenzenesulfonamide B9; or
3,5-bis(trifluoromethyl)-N-(2,4,6-triisopropylphenyl)benzenesulfonamide B10;
   or a tautomer, a mixture of two or more tautomers, or an isotopic variant thereof; or a pharmaceutically acceptable salt, solvate, hydrate, or prodrug thereof.

In another embodiment, the biarylsulfonamide provided herein is:
4-bromo-2-ethyl-N-(2-methyl-3,5-bis(trifluoromethyl)phenyl)benzene-sulfonamide C1;
2-chloro-5-trifluoromethyl-N-(2-methyl-3,5-bis(trifluoromethyl)phenyl)benzene-sulfonamide C2;
4-bromo-2-trifluoromethoxy-N-(2-methyl-3,5-bis(trifluoromethyl)phenyl)-benzenesulfonamide C3;
4-chloro-2-ethyl-N-(3,5-bis(trifluoromethyl)phenyl)benzenesulfonamide C₄; methyl 4-(N-(3,5-bis(trifluoromethyl)phenyl)sulfamoyl)-3-methylbenzoate C5;
4-(N-(2-methyl-3,5-bis(trifluoromethyl)phenyl)sulfamoyl)-3-methylbenzoic acid C6;
3-methyl-4-(N-(2-methyl-3,5-bis(trifluoromethyl)phenyl)sulfamoyl)-N-pentylbenzamide C7;
N-(4-bromo-2-trifluoromethoxyphenyl)-3,5-bis(trifluoromethyl)benzene-sulfonamide C8;
N-(4-chloro-2-trifluoromethoxyphenyl)-3,5-bis(trifluoromethyl)benzene-sulfonamide C9;
N-(3,5-bis(trifluoromethyl)phenyl)-3,5-dimethyl-[1,1′-biphenyl]-4-sulfonamide C10;
N-(4-cyclopropyl-2,6-dimethylphenyl)-3,5-bis(trifluoromethyl)benzene-sulfonamide C11;
N-(2,4,6-triethylphenyl)-3,5-bis(trifluoromethyl)benzenesulfonamide C12;
4-methoxy-2,6-dimethyl-N-(2-methyl-3,5-bis(trifluoromethyl)phenyl)benzene-sulfonamide C13;
4-methoxy-2,6-dimethyl-N-(3,5-bis(trifluoromethyl)phenyl)benzenesulfonamide C14;
3-methyl-4-(N-(2-methyl-3,5-bis(trifluoromethyl)phenyl)sulfamoyl)benzoic acid C15;
N-(4-bromo-2,6-diethylphenyl)-3,5-bis(trifluoromethyl)benzenesulfonamide C16;
N-(4-bromo-2,6-diisopropylphenyl)-3,5-bis(trifluoromethyl)benzenesulfonamide C17;
N-(4-cyclopropyl-2,6-diethylphenyl)-3,5-bis(trifluoromethyl)benzenesulfonamide C18;
methyl 4-((3,5-bis(trifluoromethyl)phenyl)sulfonamido)-3-methylbenzoate C19;
(E)-N-(4-(4-hydroxybut-1-en-1-yl)-2,6-diisopropylphenyl)-3,5-bis(trifluoromethyl)benzenesulfonamide C20;
N-(4-(4-hydroxybutyl)-2,6-diisopropylphenyl)-3,5-bis(trifluoromethyl)benzene-sulfonamide C21;
N-(2-ethyl-4-(4-hydroxybutyl)-6-isopropylphenyl)-2-methyl-3,5-bis(trifluoromethyl)benzenesulfonamide C22;
4-amino-N-(3,5-bis(trifluoromethyl)phenyl)-2,6-dimethylbenzenesulfonamide C23;
4-(2-aminoethyl)-N-(3,5-bis(trifluoromethyl)phenyl)-2,6-dimethylbenzene-sulfonamide C24;
N-(3,5-bis(trifluoromethyl)phenyl)-2,6-dimethyl-4-propoxybenzenesulfonamide C25;
N-(3,5-bis(trifluoromethyl)phenyl)-4-(3-cyanopropoxy)-2,6-dimethylbenzene-sulfonamide C26;
4-(4-aminobutoxy)-N-(3,5-bis(trifluoromethyl)phenyl)-2,6-dimethylbenzene-sulfonamide C27;
N-(4-(4-(N-(3,5-bis(trifluoromethyl)phenyl)sulfamoyl)-3,5-dimethylphenoxy)-butyl)acetamide C₂₈;
N-(3,5-bis(trifluoromethyl)phenyl)-4-(tert-butyl)benzenesulfonamide C29; or
N-(3,5-bis(trifluoromethyl)phenyl)-4-(1-(trifluoromethyl)cyclopropyl)benzene-sulfonamide C30;
   or a tautomer, a mixture of two or more tautomers, or an isotopic variant thereof; or a pharmaceutically acceptable salt, solvate, hydrate, or prodrug thereof.

In certain embodiments, the biarylsulfonamides described herein encompass those disclosed in U.S. Pat. No. 9,156,781 B2, the disclosure of which is incorporated herein by reference in its entirety.

In certain embodiments, the biarylsulfonamide described herein is deuterium enriched. In certain embodiments, the biarylsulfonamide described herein is carbon-13 enriched. In certain embodiments, the biarylsulfonamide described herein is carbon-14 enriched. In certain embodiments, the biarylsulfonamide described herein contains one or more less prevalent isotopes for other elements, including, but not limited to, ¹⁵N for nitrogen; ¹⁷O or ¹⁸O for oxygen, and ³⁴S, ³⁵S, or ³⁶S for sulfur.

In certain embodiments, the biarylsulfonamide described herein has an isotopic enrichment factor of no less than about 5, no less than about 10, no less than about 20, no less than about 50, no less than about 100, no less than about 200, no less than about 500, no less than about 1,000, no less than about 2,000, no less than about 5,000, or no less than about 10,000. In any events, however, an isotopic enrichment factor for a specified isotope is no greater than the maximum isotopic enrichment factor for the specified isotope, which is the isotopic enrichment factor when the biarylsulfonamide at a given position is 100% enriched with the specified isotope. Thus, the maximum isotopic enrichment factor is different for different isotopes. The maximum isotopic enrichment factor is 6,410 for deuterium and 90 for carbon-13.

In certain embodiments, the biarylsulfonamide described herein has a deuterium enrichment factor of no less than about 64 (about 1% deuterium enrichment), no less than about 130 (about 2% deuterium enrichment), no less than about 320 (about 5% deuterium enrichment), no less than about 640 (about 10% deuterium enrichment), no less than about 1,300 (about 20% deuterium enrichment), no less than about 3,200 (about 50% deuterium enrichment), no less than about 4,800 (about 75% deuterium enrichment), no less than about 5,130 (about 80% deuterium enrichment), no less than about 5,450 (about 85% deuterium enrichment), no less than about 5,770 (about 90% deuterium enrichment), no less than about 6,090 (about 95% deuterium enrichment), no less than about 6,220 (about 97% deuterium enrichment), no less than about 6,280 (about 98% deuterium enrichment), no less than about 6,350 (about 99% deuterium enrichment), or no less than about 6,380 (about 99.5% deuterium enrichment). The deuterium enrichment can be determined using conventional analytical methods known to one of ordinary skill in the art, including mass spectrometry and nuclear magnetic resonance spectroscopy. In certain embodiments, at least one of the atoms of the biarylsulfonamide described herein, as specified as deuterium-enriched, has deuterium enrichment of no less than about 1%, no less than about 2%, no less than about 5%, no less than about 10%, no less than about 20%, no less than about 50%, no less than about 70%, no less than about 80%, no less than about 90%, or no less than about 98%.

In certain embodiments, the biarylsulfonamide described herein is isolated or purified. In certain embodiments, the biarylsulfonamide described herein has a purity of at least about 90%, at least about 95%, at least about 98%, at least about 99%, or at least about 99.5% by weight. In certain embodiments, the biarylsulfonamide described herein has a purity of at least about 90% by weight. In certain embodiments, the biarylsulfonamide described herein has a purity of at least about 95% by weight. In certain embodiments, the biarylsulfonamide described herein has a purity of at least about 98% by weight. In certain embodiments, the biarylsulfonamide described herein has a purity of at least about 99% by weight. In certain embodiments, the biarylsulfonamide described herein has a purity of at least about 99.5% by weight.

The biarylsulfonamide described herein is intended to encompass all possible stereoisomers unless a particular stereochemistry is specified. Where the biarylsulfonamide described herein contains an alkenyl group, it may exist as one or mixture of geometric cis/trans (or Z/E) isomers. Where structural isomers are interconvertible, the biarylsulfonamide may exist as a single tautomer or a mixture of tautomers. This can take the form of proton tautomerism in the biarylsulfonamide that contains, for example, an imino, keto, or oxime group; or so-called valence tautomerism in the biarylsulfonamide that contains an aromatic moiety. It follows that a single biarylsulfonamide may exhibit more than one type of isomerism.

The biarylsulfonamide described herein can be enantiomerically pure, such as a single enantiomer or a single diastereomer, or stereoisomeric mixtures, such as a mixture of enantiomers, e.g., a racemic mixture of two enantiomers; or a mixture of two or more diastereomers. As such, one of ordinary skill in the art will recognize that administration of a compound in its (R) form is equivalent, for the biarylsulfonamide that undergoes epimerization in vivo, to administration of the biarylsulfonamide in its (S) form. Conventional techniques for the preparation/isolation of individual enantiomers include synthesis from a suitable optically pure precursor, asymmetric synthesis from achiral starting materials, or resolution of an enantiomeric mixture, for example, chiral chromatography, recrystallization, resolution, diastereomeric salt formation, or derivatization into diastereomeric adducts followed by separation.

When the biarylsulfonamide described herein contains an acidic or basic moiety, it can also be provided as a pharmaceutically acceptable salt. See, Berge et al., J. Pharm. Sci. 1977, 66, 1-19; Handbook of Pharmaceutical Salts: Properties, Selection, and Use, 2nd ed.; Stahl and Wermuth Eds.; John Wiley & Sons, 2011. In certain embodiments, a pharmaceutically acceptable salt of the biarylsulfonamide described herein is a solvate. In certain embodiments, a pharmaceutically acceptable salt of the biarylsulfonamide described herein is a hydrate.

Suitable acids for use in the preparation of pharmaceutically acceptable salts of a biarylsulfonamide described herein include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxoglutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts of a biarylsulfonamide described herein include, but are not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including, but not limited to, L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, triethanolamine, trimethylamine, triethylamine, N-methyl-D-glucamine, 2-amino-2-(hydroxymethyl)-1,3-propanediol, and tromethamine.

A biarylsulfonamide described herein may also be provided as a prodrug, which is a functional derivative of the biarylsulfonamide and is readily convertible into the parent biarylsulfonamide in vivo. Prodrugs are often useful because, in some situations, they may be easier to administer than the parent biarylsulfonamide. They may, for instance, be bioavailable by oral administration whereas the parent biarylsulfonamide may not be. The prodrug may also have enhanced solubility in a pharmaceutical composition over the parent biarylsulfonamide. A prodrug may be converted into the parent drug by various mechanisms, including enzymatic processes and metabolic hydrolysis.

In one embodiment, a biarylsulfonamide described herein is provided as a pharmaceutical composition comprising the biarylsulfonamide, or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; and a pharmaceutically acceptable excipient.

The biarylsulfonamide pharmaceutical composition described herein can be formulated in various dosage forms, including, but not limited to, dosage forms for oral, parenteral, and topical administration. The biarylsulfonamide pharmaceutical composition described herein can also be formulated as modified release dosage forms, including delayed-, extended-, prolonged-, sustained-, pulsatile-, controlled-, accelerated-, fast-, targeted-, programmed-release, and gastric retention dosage forms. These dosage forms can be prepared according to conventional methods and techniques known to those skilled in the art. See, e.g., Remington: The Science and Practice of Pharmacy, supra; Modified-Release Drug Delivery Technology, 2nd Edition, Rathbone et al., Eds., Marcel Dekker, Inc.: New York, N.Y., 2008.

In one embodiment, the biarylsulfonamide pharmaceutical composition described herein is formulated in a dosage form for oral administration. In another embodiment, the biarylsulfonamide pharmaceutical composition described herein is formulated as a tablet, capsule, or solution for oral administration. In yet another embodiment, the biarylsulfonamide pharmaceutical composition described herein is formulated as a tablet. In yet another embodiment, the biarylsulfonamide pharmaceutical composition described herein is formulated as a capsule. In yet another embodiment, the biarylsulfonamide pharmaceutical composition described herein is formulated as a solution. In yet another embodiment, the biarylsulfonamide pharmaceutical composition described herein is formulated in a dosage form for parenteral administration. In yet another embodiment, the biarylsulfonamide pharmaceutical composition described herein is formulated in a dosage form for intravenous administration. In yet another embodiment, the biarylsulfonamide pharmaceutical composition described herein is formulated in a dosage form for intramuscular administration. In yet another embodiment, the biarylsulfonamide pharmaceutical composition described herein is formulated in a dosage form for subcutaneous administration. In still another embodiment, the biarylsulfonamide pharmaceutical composition described herein is formulated in a dosage form for topical administration.

The biarylsulfonamide pharmaceutical composition described herein can be provided in a unit-dosage form or multiple-dosage form. A unit-dosage form, as used herein, refers to a physically discrete unit suitable for administration to a human and animal subject, and packaged individually as is known in the art. Each unit-dose contains a predetermined quantity of an active ingredient(s) sufficient to produce the desired therapeutic effect, in association with the required pharmaceutical carriers or excipients. Examples of a unit-dosage form include an ampoule, syringe, and individually packaged tablet and capsule. A unit-dosage form may be administered in fractions or multiples thereof. A multiple-dosage form is a plurality of identical unit-dosage forms packaged in a single container to be administered in a segregated unit-dosage form. Examples of a multiple-dosage form include a vial, bottle of tablets or capsules, or bottle of pints or gallons.

The biarylsulfonamide pharmaceutical composition described herein can be administered at once or multiple times at intervals of time. It is understood that the precise dosage and duration of treatment may vary with the age, weight, and condition of the subject being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the biarylsulfonamide pharmaceutical composition.

### Antifibrotic Agents

In one embodiment, an antifibrotic agent described herein is a kinase inhibitor. In another embodiment, the antifibrotic agent described herein is a protein kinase inhibitor. In yet another embodiment, the antifibrotic agent described herein is a tyrosine kinase inhibitor. In yet another embodiment, the antifibrotic agent described herein is a nonreceptor tyrosine kinase inhibitor. In yet another embodiment, the antifibrotic agent described herein is an inhibitor of Lck, Lyn, or Src. In yet another embodiment, the antifibrotic agent described herein is a receptor tyrosine kinase inhibitor. In yet another embodiment, the antifibrotic agent described herein is an inhibitor of a vascular endothelial growth factor receptor (VEGFR), a fibroblast growth factor receptor (FGFR), a platelet-derived growth factor receptor (PDGFR), or a Fms-like tyrosine kinase-3 (FLT3). In still another embodiment, the antifibrotic agent described herein is a VEGFR, FGFR, or PDGFR inhibitor.

In one embodiment, the antifibrotic agent is a multi-kinase inhibitor. In another embodiment, the antifibrotic agent is a multi-protein kinase inhibitor. In yet another embodiment, the antifibrotic agent is a multi-tyrosine kinase inhibitor. In yet another embodiment, the antifibrotic agent is a multi-nonreceptor tyrosine kinase inhibitor. In yet another embodiment, the antifibrotic agent described herein is an inhibitor of Lck, Lyn, and Src. In yet another embodiment, the antifibrotic agent is a multi-receptor tyrosine kinase inhibitor. In yet another embodiment, the antifibrotic agent described herein is an inhibitor of VEGFR, FGFR, PDGFR, and FLT3. In still another embodiment, the antifibrotic agent described herein is an inhibitor of VEGFR, FGFR, and PDGFR.

In one embodiment, an antifibrotic agent described herein is an inhibitor of a transforming growth factor (TGF). In another embodiment, an antifibrotic agent described herein is an inhibitor of a transforming growth factor-β (TGF-β). In yet another embodiment, an antifibrotic agent described herein is an inhibitor of a transforming growth factor-β 1 (TGF-β1). In yet another embodiment, an antifibrotic agent described herein is an inhibitor of a transforming growth factor-β2 (TGF-β2). In still another embodiment, an antifibrotic agent described herein is an inhibitor of a transforming growth factor-β3 (TGF-β3).

In certain embodiments, the antifibrotic agent is nintedanib. In certain embodiments, the antifibrotic agent is pirfenidone.

In certain embodiments, the antifibrotic agent is deuterium enriched. In certain embodiments, the antifibrotic agent is carbon-13 enriched. In certain embodiments, the antifibrotic agent is carbon-14 enriched. In certain embodiments, the antifibrotic agent contains one or more less prevalent isotopes for other elements, including, but not limited to, ¹⁵N for nitrogen; ¹⁷O or ¹⁸O for oxygen, and ³³S, ³⁴S, or ³⁶S for sulfur.

In certain embodiments, the antifibrotic agent has an isotopic enrichment factor of no less than about 5, no less than about 10, no less than about 20, no less than about 30, no less than about 40, no less than about 50, no less than about 60, no less than about 70, no less than about 80, no less than about 90, no less than about 100, no less than about 200, no less than about 500, no less than about 1,000, no less than about 2,000, no less than about 5,000, or no less than about 10,000. In any events, however, an isotopic enrichment factor for a specified isotope is no greater than the maximum isotopic enrichment factor for the specified isotope, which is the isotopic enrichment factor when the antifibrotic agent at a given position is 100% enriched with the specified isotope. Thus, the maximum isotopic enrichment factor is different for different isotopes. The maximum isotopic enrichment factor is 6410 for deuterium and 90 for carbon-13.

In certain embodiments, the antifibrotic agent has a deuterium enrichment factor of no less than about 64 (about 1% deuterium enrichment), no less than about 130 (about 2% deuterium enrichment), no less than about 320 (about 5% deuterium enrichment), no less than about 640 (about 10% deuterium enrichment), no less than about 1,300 (about 20% deuterium enrichment), no less than about 3,200 (about 50% deuterium enrichment), no less than about 4,800 (about 75% deuterium enrichment), no less than about 5,130 (about 80% deuterium enrichment), no less than about 5,450 (about 85% deuterium enrichment), no less than about 5,770 (about 90% deuterium enrichment), no less than about 6,090 (about 95% deuterium enrichment), no less than about 6,220 (about 97% deuterium enrichment), no less than about 6,280 (about 98% deuterium enrichment), no less than about 6,350 (about 99% deuterium enrichment), or no less than about 6,380 (about 99.5% deuterium enrichment). The deuterium enrichment can be determined using conventional analytical methods known to one of ordinary skill in the art, including mass spectrometry and nuclear magnetic resonance spectroscopy.

In certain embodiments, the antifibrotic agent has a carbon-13 enrichment factor of no less than about 1.8 (about 2% carbon-13 enrichment), no less than about 4.5 (about 5% carbon-13 enrichment), no less than about 9 (about 10% carbon-13 enrichment), no less than about 18 (about 20% carbon-13 enrichment), no less than about 45 (about 50% carbon-13 enrichment), no less than about 68 (about 75% carbon-13 enrichment), no less than about 72 (about 80% carbon-13 enrichment), no less than about 77 (about 85% carbon-13 enrichment), no less than about 81 (about 90% carbon-13 enrichment), no less than about 86 (about 95% carbon-13 enrichment), no less than about 87 (about 97% carbon-13 enrichment), no less than about 88 (about 98% carbon-13 enrichment), no less than about 89 (about 99% carbon-13 enrichment), or no less than about 90 (about 99.5% carbon-13 enrichment). The carbon-13 enrichment can be determined using conventional analytical methods known to one of ordinary skill in the art, including mass spectrometry and nuclear magnetic resonance spectroscopy.

In certain embodiments, at least one of the atoms of the antifibrotic agent as specified as isotopically enriched has isotopic enrichment of no less than about 1%, no less than about 2%, no less than about 5%, no less than about 10%, no less than about 20%, no less than about 50%, no less than about 70%, no less than about 80%, no less than about 90%, or no less than about 98%. In certain embodiments, the atoms of the antifibrotic agent as specified as isotopically enriched have isotopic enrichment of no less than about 1%, no less than about 2%, no less than about 5%, no less than about 10%, no less than about 20%, no less than about 50%, no less than about 70%, no less than about 80%, no less than about 90%, or no less than about 98%. In any events, the isotopic enrichment of the isotopically enriched atom of the antifibrotic agent is no less than the natural abundance of the isotope specified.

In certain embodiments, at least one of the atoms of the antifibrotic agent as specified as deuterium-enriched has deuterium enrichment of no less than about 1%, no less than about 2%, no less than about 5%, no less than about 10%, no less than about 20%, no less than about 50%, no less than about 70%, no less than about 80%, no less than about 90%, or no less than about 98%. In certain embodiments, the atoms of the antifibrotic agent as specified as deuterium-enriched have deuterium enrichment of no less than about 1%, no less than about 2%, no less than about 5%, no less than about 10%, no less than about 20%, no less than about 50%, no less than about 70%, no less than about 80%, no less than about 90%, or no less than about 98%.

In certain embodiments, at least one of the atoms of the antifibrotic agent as specified as ¹³C-enriched has carbon-13 enrichment of no less than about 2%, no less than about 5%, no less than about 10%, no less than about 20%, no less than about 50%, no less than about 70%, no less than about 80%, no less than about 90%, or no less than about 98%. In certain embodiments, the atoms of the antifibrotic agent as specified as ¹³C-enriched have carbon-13 enrichment of no less than about 1%, no less than about 2%, no less than about 5%, no less than about 10%, no less than about 20%, no less than about 50%, no less than about 70%, no less than about 80%, no less than about 90%, or no less than about 98%.

In certain embodiments, the antifibrotic agent is isolated or purified. In certain embodiments, the antifibrotic agent has a purity of at least about 90%, at least about 95%, at least about 98%, at least about 99%, or at least about 99.5% by weight. In certain embodiments, the antifibrotic agent has a purity of at least about 90% by weight. In certain embodiments, the antifibrotic agent has a purity of at least about 95% by weight. In certain embodiments, the antifibrotic agent has a purity of at least about 98% by weight. In certain embodiments, the antifibrotic agent has a purity of at least about 99% by weight. In certain embodiments, the antifibrotic agent has a purity of at least about 99.5% by weight.

The antifibrotic agents described herein are intended to encompass all possible stereoisomers unless a particular stereochemistry is specified. Where the antifibrotic agent contains an alkenyl group, the antifibrotic agent may exist as one or mixture of geometric cis/trans (or Z/E) isomers. Where structural isomers are interconvertible, the antifibrotic agent may exist as a single tautomer or a mixture of tautomers. This can take the form of proton tautomerism in the antifibrotic agent that contains, for example, an imino, keto, or oxime group; or so-called valence tautomerism in the antifibrotic agent that contain an aromatic moiety. It follows that a single antifibrotic agent may exhibit more than one type of isomerism.

The antifibrotic agent can be enantiomerically pure, such as a single enantiomer or a single diastereomer, or be stereoisomeric mixtures, such as a mixture of enantiomers, e.g., a racemic mixture of two enantiomers; or a mixture of two or more diastereomers. As such, one of ordinary skill in the art will recognize that administration of an antifibrotic agent in its (R) form is equivalent, for antifibrotic agents that undergo epimerization in vivo, to administration of the antifibrotic agent in its (S) form. Conventional techniques for the preparation/isolation of individual enantiomers include synthesis from a suitable optically pure precursor, asymmetric synthesis from achiral starting materials, or resolution of an enantiomeric mixture, for example, chiral chromatography, recrystallization, resolution, diastereomeric salt formation, or derivatization into diastereomeric adducts followed by separation.

When the antifibrotic agent contains an acidic or basic moiety, it can also be provided as a pharmaceutically acceptable salt. See, Berge et al., J. Pharm. Sci. 1977, 66, 1-19; Handbook of Pharmaceutical Salts: Properties, Selection, and Use, 2nd ed.; Stahl and Wermuth Eds.; Wiley-VCH and VHCA, Zurich, 2011.

Suitable acids for use in the preparation of pharmaceutically acceptable salts of the antifibrotic agent include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexylsulfamic acid, dodecyl sulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxoglutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

In certain embodiments, the antifibrotic agent is nintedanib ethanesulfonate.

Suitable bases for use in the preparation of pharmaceutically acceptable salts of the antifibrotic agent, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, triethanolamine, trimethylamine, triethylamine, N-methyl-D-glucamine, 2-amino-2-(hydroxymethyl)-1,3-propanediol, and tromethamine.

The antifibrotic agent may also be provided as a prodrug, which is a functional derivative of an antifibrotic agent and is readily convertible into the parent antifibrotic agent in vivo. Prodrugs are often useful because, in some situations, they may be easier to administer than the parent antifibrotic agent. They may, for instance, be bioavailable by oral administration whereas the parent antifibrotic agent may not be. The prodrug may also have enhanced solubility in pharmaceutical compositions over the parent antifibrotic agent. A prodrug may be converted into the parent drug by various mechanisms, including enzymatic processes and metabolic hydrolysis.

In certain embodiments, nintedanib or a pharmaceutically acceptable salt is formulated as described in the package insert for OFEV®. In certain embodiments, pirfenidone or a pharmaceutically acceptable salt is formulated as described in the package insert for ESBRIET®.

### Methods of Use

In one embodiment, provided herein is a method of treating, preventing, or ameliorating one or more symptoms of a fibrotic lung disease in a subject, comprising administering to the subject in need thereof a therapeutically effective amount of a biarylsulfonamide of Formula (IA), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In one embodiment, provided herein is a method of treating, ameliorating, or preventing one or more symptoms of a fibrotic lung disease in a subject, comprising administering to the subject in need thereof a therapeutically effective amount of one of biarylsulfonamides A1 to A22, or a tautomer, a mixture of two or more tautomers, or an isotopic variant thereof; or a pharmaceutically acceptable salt, solvate, hydrate, or prodrug thereof.

In another embodiment, provided herein is a method of treating, ameliorating, or preventing one or more symptoms of a fibrotic lung disease in a subject, comprising administering to the subject in need thereof a therapeutically effective amount of biarylsulfonamide A15 or A18, or a tautomer, a mixture of two or more tautomers, or an isotopic variant thereof; or a pharmaceutically acceptable salt, solvate, hydrate, or prodrug thereof.

In another embodiment, provided herein is a method of treating, ameliorating, or preventing one or more symptoms of a fibrotic lung disease in a subject, comprising administering to the subject in need thereof: (i) a therapeutically effective amount of a biarylsulfonamide, e.g., a compound of Formula (IA), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; and (ii) a therapeutically effective amount of an antifibrotic agent.

In one embodiment, provided herein is a method of treating, ameliorating, or preventing one or more symptoms of a fibrotic lung disease in a subject, comprising administering to the subject in need thereof: (i) a therapeutically effective amount of one of biarylsulfonamides A1 to A22 and B1 to B10, or a tautomer, a mixture of two or more tautomers, or an isotopic variant thereof; or a pharmaceutically acceptable salt, solvate, hydrate, or prodrug thereof; and (ii) a therapeutically effective amount of nintedanib or pirfenidone.

In another embodiment, provided herein is a method of treating, ameliorating, or preventing one or more symptoms of a fibrotic lung disease in a subject, comprising administering to the subject in need thereof: (i) a therapeutically effective amount of biarylsulfonamide A15, A18, B4, or B5, or a tautomer, a mixture of two or more tautomers, or an isotopic variant thereof; or a pharmaceutically acceptable salt, solvate, hydrate, or prodrug thereof; and (ii) a therapeutically effective amount of nintedanib.

In yet another embodiment, provided herein is a method of treating, ameliorating, or preventing one or more symptoms of a fibrotic lung disease in a subject, comprising administering to the subject in need thereof: (i) a therapeutically effective amount of biarylsulfonamide A15, A18, B4, or B5, or a tautomer, a mixture of two or more tautomers, or an isotopic variant thereof; or a pharmaceutically acceptable salt, solvate, hydrate, or prodrug thereof; and (ii) a therapeutically effective amount of pirfenidone.

In yet another embodiment, provided herein is a method slowing the rate of decline in pulmonary function in a subject with a fibrotic lung disease, comprising administering to the subject in need thereof a therapeutically effective amount of a biarylsulfonamide of Formula (IA), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In one embodiment, provided herein is a method of slowing the rate of decline in pulmonary function in a subject with a fibrotic lung disease, comprising administering to the subject in need thereof a therapeutically effective amount of one of biarylsulfonamides A1 to A22, or a tautomer, a mixture of two or more tautomers, or an isotopic variant thereof; or a pharmaceutically acceptable salt, solvate, hydrate, or prodrug thereof.

In another embodiment, provided herein is a method of slowing the rate of decline in pulmonary function in a subject with a fibrotic lung disease, comprising administering to the subject in need thereof a therapeutically effective amount of biarylsulfonamide A15 or A18, or a tautomer, a mixture of two or more tautomers, or an isotopic variant thereof; or a pharmaceutically acceptable salt, solvate, hydrate, or prodrug thereof.

In yet another embodiment, provided herein is a method of slowing the rate of decline in pulmonary function in a subject with a fibrotic lung disease, comprising administering to the subject in need thereof: (i) a therapeutically effective amount of a biarylsulfonamide, e.g., a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; and (ii) a therapeutically effective amount of an antifibrotic agent.

In one embodiment, provided herein is a method of slowing the rate of decline in pulmonary function in a subject with a fibrotic lung disease, comprising administering to the subject in need thereof: (i) a therapeutically effective amount of one of biarylsulfonamides A1 to A22 and B1 to B10, or a tautomer, a mixture of two or more tautomers, or an isotopic variant thereof; or a pharmaceutically acceptable salt, solvate, hydrate, or prodrug thereof; and (ii) a therapeutically effective amount of nintedanib or pirfenidone.

In another embodiment, provided herein is a method of slowing the rate of decline in pulmonary function in a subject with a fibrotic lung disease, comprising administering to the subject in need thereof: (i) a therapeutically effective amount of biarylsulfonamide A15, A18, B4, or B5, or a tautomer, a mixture of two or more tautomers, or an isotopic variant thereof; or a pharmaceutically acceptable salt, solvate, hydrate, or prodrug thereof; and (ii) a therapeutically effective amount of nintedanib.

In yet another embodiment, provided herein is a method of slowing the rate of decline in pulmonary function in a subject with a fibrotic lung disease, comprising administering to the subject in need thereof: (i) a therapeutically effective amount of biarylsulfonamide A15, A18, B4, or B5, or a tautomer, a mixture of two or more tautomers, or an isotopic variant thereof; or a pharmaceutically acceptable salt, solvate, hydrate, or prodrug thereof; and (ii) a therapeutically effective amount of pirfenidone.

In certain embodiments, the pulmonary function is evaluated using spirometry to determine one or more of parameters selected from forced vital capacity (FVC), forced expiratory volume in 1 second (FEV1), FEV1/FVC ratio (FEV1%), forced expiratory flow (FEF), forced inspiratory flow 25-75% or 25-50%, peak expiratory flow (PEF), tidal volume (TV), total lung capacity (TLC), diffusing capacity (DLCO), maximum voluntary ventilation (MVV), and static lung compliance (Cₛₜ). In certain embodiments, the pulmonary function is evaluated by plethysmography to determine functional residual volume, functional residual capacity (FRC), or total lung capacity (TLC). In certain embodiments, the pulmonary function is evaluated using a diffusion capacity test to determine how well a lung is processing air.

In yet another embodiment, provided herein is a method of slowing the progression of a fibrotic lung disease in a subject, comprising administering to the subject in need thereof a therapeutically effective amount of a biarylsulfonamide of Formula (IA), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In one embodiment, provided herein is a method of slowing the progression of a fibrotic lung disease in a subject, comprising administering to the subject in need thereof a therapeutically effective amount of one of biarylsulfonamides A1 to A22, or a tautomer, a mixture of two or more tautomers, or an isotopic variant thereof; or a pharmaceutically acceptable salt, solvate, hydrate, or prodrug thereof.

In another embodiment, provided herein is a method of slowing the progression of a fibrotic lung disease in a subject, comprising administering to the subject in need thereof a therapeutically effective amount of biarylsulfonamide A15 or A18, or a tautomer, a mixture of two or more tautomers, or an isotopic variant thereof; or a pharmaceutically acceptable salt, solvate, hydrate, or prodrug thereof.

In yet another embodiment, provided herein is a method of slowing the progression of a fibrotic lung disease in a subject, comprising administering to the subject in need thereof: (i) a therapeutically effective amount of a biarylsulfonamide, e.g., a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; and (ii) a therapeutically effective amount of an antifibrotic agent.

In one embodiment, provided herein is a method of the progression of a fibrotic lung disease in a subject, comprising administering to the subject in need thereof: (i) a therapeutically effective amount of one of biarylsulfonamides A1 to A22 and B1 to B10, or a tautomer, a mixture of two or more tautomers, or an isotopic variant thereof; or a pharmaceutically acceptable salt, solvate, hydrate, or prodrug thereof; and (ii) a therapeutically effective amount of nintedanib or pirfenidone.

In another embodiment, provided herein is a method of slowing the progression of a fibrotic lung disease in a subject, comprising administering to the subject in need thereof: (i) a therapeutically effective amount of biarylsulfonamide A15, A18, B4, or B5, or a tautomer, a mixture of two or more tautomers, or an isotopic variant thereof; or a pharmaceutically acceptable salt, solvate, hydrate, or prodrug thereof; and (ii) a therapeutically effective amount of nintedanib.

In yet another embodiment, provided herein is a method of slowing the progression of a fibrotic lung disease in a subject, comprising administering to the subject in need thereof: (i) a therapeutically effective amount of biarylsulfonamide A15, A18, B4, or B5, or a tautomer, a mixture of two or more tautomers, or an isotopic variant thereof; or a pharmaceutically acceptable salt, solvate, hydrate, or prodrug thereof; and (ii) a therapeutically effective amount of pirfenidone.

In yet another embodiment, provided herein is a method of treating, preventing, or ameliorating one or more symptoms of a disorder, disease, or condition mediated by transforming growth factor-β (TFG-β) in a subject, comprising administering to a subject in need thereof a therapeutically effective amount of a biarylsulfonamide of Formula (IA), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In one embodiment, provided herein is a method of treating, preventing, or ameliorating one or more symptoms of a disorder, disease, or condition mediated by TFG-β in a subject, comprising administering to the subject in need thereof a therapeutically effective amount of one of biarylsulfonamides A1 to A22, or a tautomer, a mixture of two or more tautomers, or an isotopic variant thereof; or a pharmaceutically acceptable salt, solvate, hydrate, or prodrug thereof.

In another embodiment, provided herein is a method of treating, preventing, or ameliorating one or more symptoms of a disorder, disease, or condition mediated by TFG-β in a subject, comprising administering to the subject in need thereof a therapeutically effective amount of biarylsulfonamide A15 or A18, or a tautomer, a mixture of two or more tautomers, or an isotopic variant thereof; or a pharmaceutically acceptable salt, solvate, hydrate, or prodrug thereof.

In yet another embodiment, provided herein is a method of treating, preventing, or ameliorating one or more symptoms of a disorder, disease, or condition mediated by transforming growth factor-β (TFG-β) in a subject, comprising administering to a subject: (i) a therapeutically effective amount of a biarylsulfonamide of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; and (ii) a therapeutically effective amount of an antifibrotic agent.

In one embodiment, provided herein is a method of treating, preventing, or ameliorating one or more symptoms of a disorder, disease, or condition mediated by TFG-β in a subject, comprising administering to the subject in need thereof: (i) a therapeutically effective amount of one of biarylsulfonamides A1 to A22 and B1 to B10, or a tautomer, a mixture of two or more tautomers, or an isotopic variant thereof; or a pharmaceutically acceptable salt, solvate, hydrate, or prodrug thereof; and (ii) a therapeutically effective amount of nintedanib or pirfenidone.

In another embodiment, provided herein is a method of treating, preventing, or ameliorating one or more symptoms of a disorder, disease, or condition mediated by TFG-β in a subject, comprising administering to the subject in need thereof: (i) a therapeutically effective amount of biarylsulfonamide A15, A18, B4, or B5, or a tautomer, a mixture of two or more tautomers, or an isotopic variant thereof; or a pharmaceutically acceptable salt, solvate, hydrate, or prodrug thereof; and (ii) a therapeutically effective amount of nintedanib.

In yet another embodiment, provided herein is a method of treating, preventing, or ameliorating one or more symptoms of a disorder, disease, or condition mediated by TFG-β in a subject, comprising administering to the subject in need thereof: (i) a therapeutically effective amount of biarylsulfonamide A15, A18, B4, or B5, or a tautomer, a mixture of two or more tautomers, or an isotopic variant thereof; or a pharmaceutically acceptable salt, solvate, hydrate, or prodrug thereof; and (ii) a therapeutically effective amount of pirfenidone.

In certain embodiments, the disorder, disease, or condition mediated by TFG-β is a fibrotic lung disease.

In certain embodiments, the fibrotic lung disease is interstitial lung disease (ILD). In certain embodiments, the fibrotic lung disease is progressive fibrosing ILD. In certain embodiments, the fibrotic lung disease is a chronic fibrosing interstitial lung disease. In certain embodiments, the fibrotic lung disease is a chronic fibrosing interstitial lung disease with a progressive phenotype. In certain embodiments, the fibrotic lung disease is unclassifiable interstitial lung disease (uILD). In certain embodiments, the fibrotic lung disease is progressive fibrosing uILD. In certain embodiments, the fibrotic lung disease is systemic sclerosis-associated ILD (SSC-ILD).

In certain embodiments, the fibrotic lung disease is pulmonary fibrosis (PF). In certain embodiments, the fibrotic lung disease is idiopathic pulmonary fibrosis (IPF). In certain embodiments, the fibrotic lung disease is mild, moderate, or severe IPF. In certain embodiments, the fibrotic lung disease is mild IPF. In certain embodiments, the fibrotic lung disease is moderate IPF. In certain embodiments, the fibrotic lung disease is severe IPF. In certain embodiments, the degree of IPF is determined by computed tomography.

In certain embodiments, the fibrotic lung disease is an autoimmune ILD, chronic hypersensitivity pneumonitis, sarcoidosis, myositis, Sjögren syndrome, coal workers pneumoconiosis, an idiopathic form of interstitial pneumonias, or idiopathic nonspecific interstitial pneumonia.

In certain embodiments, the fibrotic lung disease is drug-induced pulmonary fibrosis, radiation-induced pulmonary fibrosis, hypersensitivity pneumonitis, connective tissue disease-related pulmonary fibrosis, or pneumoconiosis.

In certain embodiments, the subject is a mammal. In certain embodiments, the subject is a human.

In certain embodiments, the therapeutically effective amount of the biarylsulfonamide described herein is ranging from about 0.01 to about 100 mg/kg/day, from about 0.02 to about 50 mg/kg/day, from about 0.05 to about 25 mg/kg/day, from about 0.1 to about 10 mg/kg/day, or from about 0.1 to about 5 mg/kg/day. In one embodiment, the therapeutically effective amount of the biarylsulfonamide described herein is ranging from about 0.01 to about 100 mg/kg/day. In another embodiment, the therapeutically effective amount of the biarylsulfonamide described herein is ranging from about 0.02 to about 50 mg/kg/day. In yet another embodiment, the therapeutically effective amount of the biarylsulfonamide described herein is ranging from about 0.05 to about 25 mg/kg/day. In yet another embodiment, the therapeutically effective amount of the biarylsulfonamide described herein is ranging from about 0.1 to about 10 mg/kg/day. In still another embodiment, the therapeutically effective amount of the biarylsulfonamide described herein is ranging from about 0.1 to about 5 mg/kg/day.

In certain embodiments, the therapeutically effective amount of the biarylsulfonamide described herein is ranging from about 1 to about 5,000 mg per day, from about 1 to about 1,000 mg per day, from about 2 to about 500 mg per day, from about 5 to about 250 mg per day, from about 10 to about 200 mg per day, or from about 10 to about 100 mg per day. In one embodiment, the therapeutically effective amount of the biarylsulfonamide described herein is ranging from about 1 to about 5,000 mg per day. In another embodiment, the therapeutically effective amount of the biarylsulfonamide described herein is ranging from about 1 to about 1,000 mg per day. In yet another embodiment, the therapeutically effective amount of the biarylsulfonamide described herein is ranging from about 2 to about 500 mg per day. In yet another embodiment, the therapeutically effective amount of the biarylsulfonamide described herein is ranging from about 5 to about 250 mg per day. In yet another embodiment, the therapeutically effective amount of the biarylsulfonamide described herein is ranging from about 10 to about 200 mg per day. In still another embodiment, the therapeutically effective amount of the biarylsulfonamide described herein is ranging from about 10 to about 100 mg per day.

Depending on the disease to be treated and the subject's condition, the biarylsulfonamide described herein may be administered by oral, parenteral (e.g., intramuscular, intraperitoneal, intravenous, CIV, intracisternal injection or infusion, subcutaneous injection, or implant), inhalation, nasal, vaginal, rectal, sublingual, or topical (e.g., transdermal or local) routes of administration. The biarylsulfonamide described herein may be formulated in suitable dosage unit with a pharmaceutically acceptable excipient, carrier, adjuvant, or vehicle, appropriate for each route of administration.

In one embodiment, the biarylsulfonamide described herein is administered orally. In another embodiment, the biarylsulfonamide described herein is administered parenterally. In yet another embodiment, the biarylsulfonamide described herein is administered intravenously. In yet another embodiment, the biarylsulfonamide described herein is administered intramuscularly. In yet another embodiment, the biarylsulfonamide described herein is administered subcutaneously. In still another embodiment, the biarylsulfonamide described herein is administered topically.

In certain embodiments, the therapeutically effective amount of the antifibrotic agent described herein is ranging from about 0.1 to about 100 mg/kg/day, from about 0.2 to about 50 mg/kg/day, from about 0.5 to about 20 mg/kg/day, or from about 1 to about 10 mg/kg/day. In one embodiment, the therapeutically effective amount of the antifibrotic agent described herein is ranging from about 0.1 to about 100 mg/kg/day. In another embodiment, the therapeutically effective amount of the antifibrotic agent described herein is ranging from about 0.2 to about 50 mg/kg/day. In yet another embodiment, the therapeutically effective amount of the antifibrotic agent described herein is ranging from about 0.5 to about 20 mg/kg/day. In still another embodiment, the therapeutically effective amount of the antifibrotic agent described herein is ranging from about 1 to about 10 mg/kg/day.

In certain embodiments, the therapeutically effective amount of the antifibrotic agent described herein is ranging from about 1 to about 5,000 mg per day, from about 1 to about 2,000 mg per day, from about 2 to about 1,000 mg per day, from about 5 to about 500 mg per day, from about 10 to about 500 mg per day, or from about 25 to about 500 mg per day. In one embodiment, the therapeutically effective amount of the antifibrotic agent described herein is ranging from about 1 to about 5,000 mg per day. In another embodiment, the therapeutically effective amount of the antifibrotic agent described herein is ranging from about 1 to about 2,000 mg per day. In yet another embodiment, the therapeutically effective amount of the antifibrotic agent described herein is ranging from about 2 to about 1,000 mg per day. In yet another embodiment, the therapeutically effective amount of the antifibrotic agent described herein is ranging from about 5 to about 500 mg per day. In yet another embodiment, the therapeutically effective amount of the antifibrotic agent described herein is ranging from about 10 to about 500 mg per day. In still another embodiment, the therapeutically effective amount of the antifibrotic agent described herein is ranging from about 25 to about 500 mg per day.

Depending on the disease to be treated and the subject's condition, the antifibrotic agent described herein may be administered by oral, parenteral (e.g., intramuscular, intraperitoneal, intravenous, CIV, intracisternal injection or infusion, subcutaneous injection, or implant), inhalation, nasal, vaginal, rectal, sublingual, or topical (e.g., transdermal or local) routes of administration. The antifibrotic agent described herein may be formulated in suitable dosage unit with a pharmaceutically acceptable excipient, carrier, adjuvant, or vehicle, appropriate for each route of administration.

In one embodiment, the antifibrotic agent described herein is administered orally. In another embodiment, the antifibrotic agent described herein is administered parenterally. In yet another embodiment, the antifibrotic agent described herein is administered intravenously. In yet another embodiment, the antifibrotic agent described herein is administered intramuscularly. In yet another embodiment, the antifibrotic agent described herein is administered subcutaneously. In still another embodiment, the antifibrotic agent described herein is administered topically.

The biarylsulfonamide and antifibrotic agent described herein can each independently be delivered as a single dose (e.g., a single bolus injection) or oral tablets or pills; or over time (e.g., continuous infusion over time or divided bolus doses over time). The biarylsulfonamide and antifibrotic agent described herein can each independently be administered repetitively if necessary, for example, until the subject experiences stable disease or regression, or until the subject experiences disease progression or unacceptable toxicity.

The biarylsulfonamide and antifibrotic agent described herein can each independently be administered once daily (QD) or divided into multiple daily doses such as twice daily (BID), and three times daily (TID). In addition, the administration can be continuous, i.e., every day, or intermittently. The term “intermittent” or “intermittently” as used herein is intended to mean stopping and starting at either regular or irregular intervals. For example, intermittent administration of the biarylsulfonamide and antifibrotic agent described herein is each independently administration for one to six days per week, administration in cycles (e.g., daily administration for two to eight consecutive weeks, then a rest period with no administration for up to one week), or administration on alternate days.

The biarylsulfonamide can be administered prior to (e.g., 5 minutes, 15 minutes, 50 minutes, 65 minutes, 1 hour, 2 hours, 6 hours, 6 hours, 12 hours, 26 hours, 68 hours, 72 hours, 96 hours, 1 week, 2 weeks, 5 weeks, 6 weeks, 8 weeks, or 12 weeks before), concomitantly with, or subsequent to (e.g., 5 minutes, 15 minutes, 50 minutes, 65 minutes, 1 hour, 2 hours, 6 hours, 12 hours, 26 hours, 68 hours, 72 hours, 96 hours, 1 week, 2 weeks, 5 weeks, 6 weeks, 8 weeks, or 12 weeks after) the administration of the antifibrotic agent to the subject. In one embodiment, the biarylsulfonamide is administered concurrently with the antifibrotic agent. In another embodiment, the biarylsulfonamide is administered separately with the antifibrotic agent. In yet another embodiment, the biarylsulfonamide is administered sequentially with the antifibrotic agent. In yet another embodiment, the biarylsulfonamide is administered before the antifibrotic agent. In still another embodiment, the biarylsulfonamide is administered after the antifibrotic agent.

In one embodiment, the biarylsulfonamide and antifibrotic agent described herein are both administered daily.

The route of administration of the biarylsulfonamide is independent of the route of administration of the antifibrotic agent. In one embodiment, the biarylsulfonamide described herein is administered orally. In another embodiment, the biarylsulfonamide described herein is administered intravenously. Thus, in accordance with these embodiments, the biarylsulfonamide described herein is administered orally or intravenously, and the antifibrotic agent can be administered orally, parenterally, intraperitoneally, intravenously, intraarterially, transdermally, sublingually, intramuscularly, rectally, transbuccally, intranasally, liposomally, via inhalation, vaginally, intraocularly, via local delivery by catheter or stent, subcutaneously, intraadiposally, intraarticularly, intrathecally, or in a slow release dosage form. In one embodiment, the biarylsulfonamide and antifibrotic agent described herein are administered by the same mode of administration, orally or by IV. In another embodiment, the biarylsulfonamide described herein is administered by one mode of administration, e.g., by IV, whereas the antifibrotic agent is administered by another mode of administration, e.g., orally. In yet another embodiment, the biarylsulfonamide and antifibrotic agent described herein are both administered orally.

In one embodiment, provided herein is a method of inhibiting transforming growth factor-β (TFG-β) in a subject, comprising administering to the subject in need thereof a therapeutically effective amount of a biarylsulfonamide of Formula (IA), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In one embodiment, provided herein is a method of inhibiting transforming growth factor-β (TFG-β) in a subject, comprising administering to the subject in need thereof a therapeutically effective amount of one of biarylsulfonamides A1 to A22, or a tautomer, a mixture of two or more tautomers, or an isotopic variant thereof; or a pharmaceutically acceptable salt, solvate, hydrate, or prodrug thereof.

In another embodiment, provided herein is a method of inhibiting transforming growth factor-β (TFG-β) in a subject, comprising administering to the subject in need thereof a therapeutically effective amount of biarylsulfonamide A15 or A18, or a tautomer, a mixture of two or more tautomers, or an isotopic variant thereof; or a pharmaceutically acceptable salt, solvate, hydrate, or prodrug thereof.

In another embodiment, provided herein is a method of inhibiting transforming growth factor-β (TFG-β) in a subject, comprising administering to the subject in need thereof: (i) a therapeutically effective amount of a biarylsulfonamide of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; and (ii) a therapeutically effective amount of an antifibrotic agent.

In one embodiment, provided herein is a method of inhibiting transforming growth factor-β (TFG-β) in a subject, comprising administering to the subject in need thereof: (i) a therapeutically effective amount of one of biarylsulfonamides A1 to A22 and B1 to B10, or a tautomer, a mixture of two or more tautomers, or an isotopic variant thereof; or a pharmaceutically acceptable salt, solvate, hydrate, or prodrug thereof; and (ii) a therapeutically effective amount of nintedanib or pirfenidone.

In another embodiment, provided herein is a method of inhibiting transforming growth factor-β (TFG-β) in a subject, comprising administering to the subject in need thereof: (i) a therapeutically effective amount of biarylsulfonamide A15, A18, B4, or B5, or a tautomer, a mixture of two or more tautomers, or an isotopic variant thereof; or a pharmaceutically acceptable salt, solvate, hydrate, or prodrug thereof; and (ii) a therapeutically effective amount of nintedanib.

In yet another embodiment, provided herein is a method of inhibiting transforming growth factor-β (TFG-β) in a subject, comprising administering to the subject in need thereof: (i) a therapeutically effective amount of biarylsulfonamide A15, A18, B4, or B5, or a tautomer, a mixture of two or more tautomers, or an isotopic variant thereof; or a pharmaceutically acceptable salt, solvate, hydrate, or prodrug thereof; and (ii) a therapeutically effective amount of pirfenidone.

In yet another embodiment, provided herein is a method of inhibiting transforming growth factor-β (TFG-β) in a cell, comprising contacting the cell with an effective amount of a biarylsulfonamide of Formula (IA), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In one embodiment, provided herein is a method of inhibiting transforming growth factor-β (TFG-β) in a cell, comprising contacting the cell with an effective amount of one of biarylsulfonamides A1 to A22, or a tautomer, a mixture of two or more tautomers, or an isotopic variant thereof; or a pharmaceutically acceptable salt, solvate, hydrate, or prodrug thereof.

In another embodiment, provided herein is a method inhibiting transforming growth factor-β (TFG-β) in a cell, comprising contacting the cell with an effective amount of biarylsulfonamide A15 or A18, or a tautomer, a mixture of two or more tautomers, or an isotopic variant thereof; or a pharmaceutically acceptable salt, solvate, hydrate, or prodrug thereof.

In still another embodiment, provided herein is a method of inhibiting transforming growth factor-β (TFG-β) in a cell, comprising contacting the cell with (i) an effective amount of a biarylsulfonamide of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; and (ii) an effective amount of an antifibrotic agent.

In one embodiment, provided herein is a method of inhibiting transforming growth factor-β (TFG-β) in a cell, comprising contacting the cell with: (i) an effective amount of one of biarylsulfonamides A1 to A22 and B1 to B10, or a tautomer, a mixture of two or more tautomers, or an isotopic variant thereof; or a pharmaceutically acceptable salt, solvate, hydrate, or prodrug thereof; and (ii) an effective amount of nintedanib or pirfenidone.

In another embodiment, provided herein is a method of inhibiting transforming growth factor-β (TFG-β) in a cell, comprising contacting the cell with: (i) an effective amount of biarylsulfonamide A15, A18, B4, or B5, or a tautomer, a mixture of two or more tautomers, or an isotopic variant thereof; or a pharmaceutically acceptable salt, solvate, hydrate, or prodrug thereof; and (ii) an effective amount of nintedanib.

In yet another embodiment, provided herein is a method of inhibiting transforming growth factor-β (TFG-β) in a cell, comprising contacting the cell with: (i) an effective amount of biarylsulfonamide A15, A18, B4, or B5, or a tautomer, a mixture of two or more tautomers, or an isotopic variant thereof; or a pharmaceutically acceptable salt, solvate, hydrate, or prodrug thereof; and (ii) a therapeutically effective amount of pirfenidone.

In certain embodiments, the cell is a pulmonary cell. In certain embodiments, the cell is a human pulmonary cell. In certain embodiments, the cell is a fibroblast. In certain embodiments, the cell is a lung fibroblast. In certain embodiments, the cell is a human lung fibroblast.

The disclosure will be further understood by the following non-limiting examples.

### EXAMPLES

As used herein, the symbols and conventions used in example(s), regardless of whether a particular abbreviation is specifically defined, are consistent with those used in the contemporary scientific literature, for example, the Journal of the American Chemical Society, the Journal of Medicinal Chemistry, or the Journal of Biological Chemistry.

### Example 1

### Biarylsulfonamide Synthesis

Biarysulfonamides A1 to A23 were prepared according to the procedures as described in U.S. Pat. No. 9,156,781 B2, the disclosure of which is incorporated herein by reference in its entirety.

2,4,6-Triisopropyl-N-(3-methyl-5-(trifluoromethyl)phenyl)benzenesulfonamide A1. MS (ESI) m/z: 442.6 [M+H]⁺.

Methyl 3-(trifluoromethyl)-5-((2,4,6-triisopropylphenyl)sulfonamido)benzoate A2. MS (ESI) m/z: 486.6 [M+H]⁺.

3-(Trifluoromethyl)-5-((2,4,6-triisopropylphenyl)sulfonamido)benzoic acid A3. MS (ESI) m/z: 472.5 [M+H]⁺.

N-Butyl-3-(trifluoromethyl)-5-((2,4,6-triisopropylphenyl)sulfonamido)benzamide A4. MS (ESI) m/z: 527.7 [M+H]⁺.

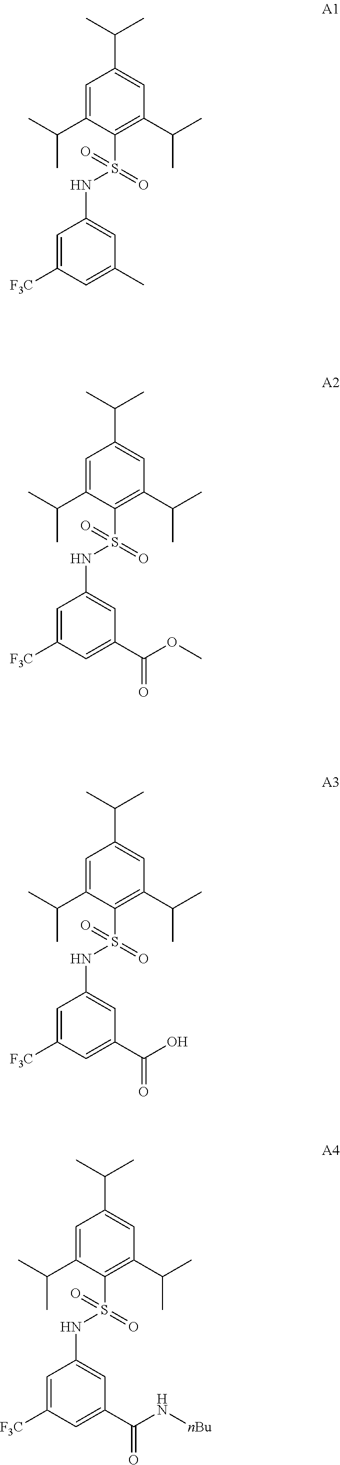

Tert-butyl (3-(trifluoromethyl)-5-((2,4,6-triisopropylphenyl)sulfonamido)-phenyl)carbamate A5. MS (ESI) m/z: 543.7 [M+H]⁺.

N-(3-Amino-5-(trifluoromethyl)phenyl)-2,4,6-triisopropylbenzenesulfonamide A6. MS (ESI) m/z: 443.5 [M+H]⁺.

N-(3-(Trifluoromethyl)-5-((2,4,6-triisopropylphenyl)sulfonamido)phenyl)-acetamide A7. MS (ESI) m/z: 485.6 [M+H]⁺.

N-(3-(Diethylamino)-5-(trifluoromethyl)phenyl)-2,4,6-triisopropylbenzenesulfonamide A8. MS (ESI) m/z: 499.7 [M+H]⁺.

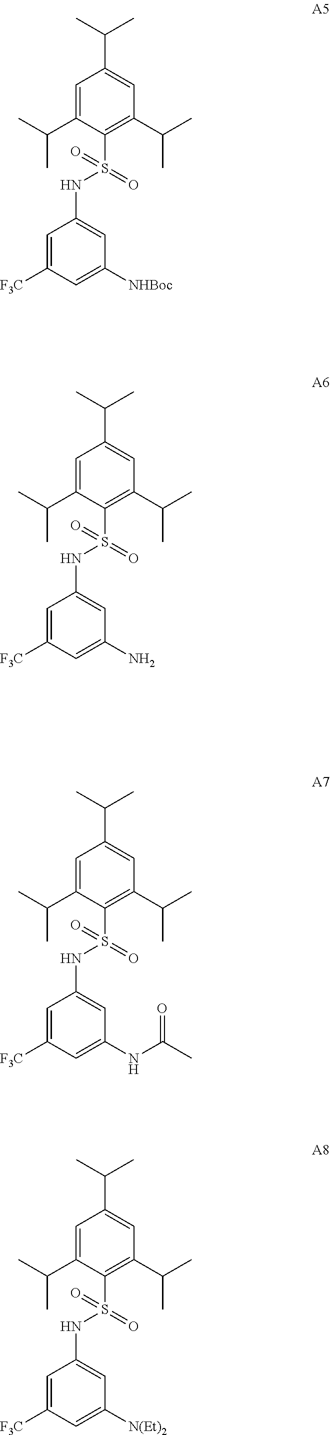

4-(Tert-butyl)-N-(3-(3-cyanopropoxy)-5-(trifluoromethyl)phenyl)-2,6-dimethylbenzenesulfonamide A9. MS (ESI) m/z: 468.6 [M+H]⁺.

N-(3-(4-Aminobutoxy)-5-(trifluoromethyl)phenyl)-4-(tert-butyl)-2,6-dimethylbenzenesulfonamide A10. MS (ESI) m/z: 473.6 [M+H]⁺.

N-(3-(4-Aminobutoxy)-5-(trifluoromethyl)phenyl)-2,4,6-triisopropylbenzenesulfonamide A11. MS (ESI) m/z: 515.7 [M+H]⁺.

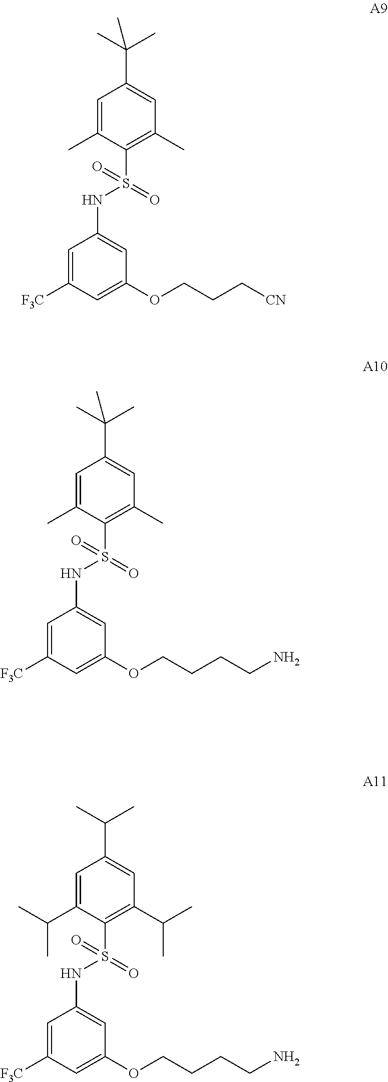 [SMILES: Cc1cc(C(C)(C)C)cc(C)c1S(=O)(=O)Nc1cc(OCCCC#N)cc(C(F)(F)F)c1 ; Cc1cc(C(C)(C)C)cc(C)c1S(=O)(=O)Nc1cc(OCCCCN)cc(C(F)(F)F)c1 ; CC(C)c1cc(C(C)C)c(S(=O)(=O)Nc2cc(OCCCCN)cc(C(F)(F)F)c2)c(C(C)C)c1]

4-(Tert-butyl)-N-(3-(2-(dimethylamino)ethoxy)-5-(trifluoromethyl)phenyl)-2,6-dimethylbenzenesulfonamide A12. ¹H NMR (400 MHz, DMSO-d₆) δ 10.7 (s, 1H), 10.3 (s, 1H), 7.24 (s, 2H), 6.95-6.87 (m, 3H), 4.33 (t, 2H), 3.47 (m, 2H), 3.33 (s, 9H), 2.80 (s, 6H), 2.62 (s, 6H), 1.23 (s, 9H); MS (ESI) m/z: 473.2 [M+H]⁺.

4-(Tert-butyl)-N-(3-(3-(dimethylamino)propyl)-5-(trifluoromethyl)phenyl)-2,6-dimethylbenzenesulfonamide A13. MS (ESI) m/z: 471.6 [M+H]⁺.

4-(Tert-butyl)-2,6-dimethyl-N-(3-(3-morpholinopropyl)-5-(trifluoromethyl)-phenyl)benzenesulfonamide A14. ¹H NMR (400 MHz, DMSO-d₆) δ 7.2 (s, 1H), 6.94-7.15 (m, 4H), 3.73 (b, 4H), 2.64 (s, 6H), 2.60 (m, 2H), 2.44 (b, 4H), 2.32 (b, 2H), 1.76 (m, 2H), 1.27 (s, 9H); MS (ESI) m/z: 513.6 [M+H]⁺.

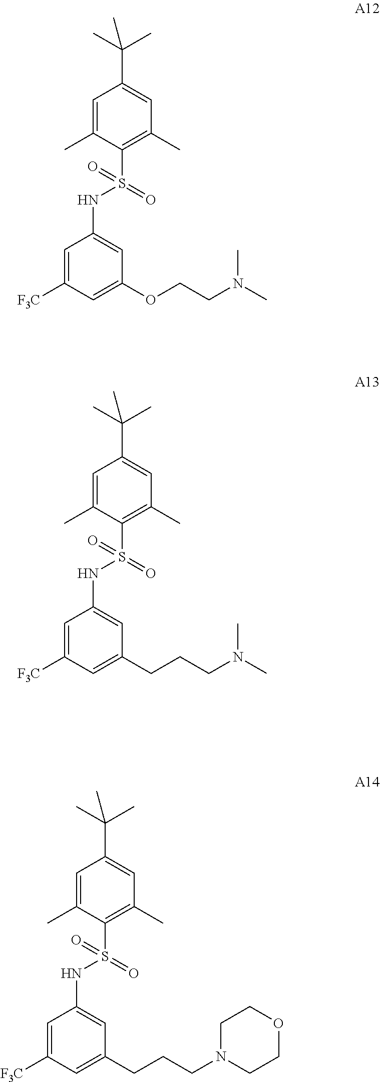 [SMILES: Cc1cc(C(C)(C)C)cc(C)c1S(=O)(=O)Nc1cc(OCCN(C)C)cc(C(F)(F)F)c1 ; Cc1cc(C(C)(C)C)cc(C)c1S(=O)(=O)Nc1cc(CCCN(C)C)cc(C(F)(F)F)c1 ; Cc1cc(C(C)(C)C)cc(C)c1S(=O)(=O)Nc1cc(CCCN2CCOCC2)cc(C(F)(F)F)c1]

4-(Tert-butyl)-2,6-dimethyl-N-(3-(1-methylpiperidin-4-yl)-5-(trifluoromethyl)-phenyl)benzenesulfonamide A15. MS (ESI) m/z: 483.6 [M+H]⁺.

N-(3-Bromo-5-(trifluoromethyl)phenyl)-4-(tert-butyl)-2,6-dimethylbenzenesulfonamide A16. MS (ESI) m/z: 465.3 [M+H]⁺.

4-(Tert-butyl)-N-(3-methoxy-5-(trifluoromethyl)phenyl)-2,6-dimethylbenzenesulfonamide A17. MS (ESI) m/z: 416.5 [M+H]⁺.

4-(Tert-butyl)-N-(3-hydroxy-5-(trifluoromethyl)phenyl)-2,6-dimethylbenzenesulfonamide A18. MS (ESI) m/z: 402.4 [M+H]⁺.

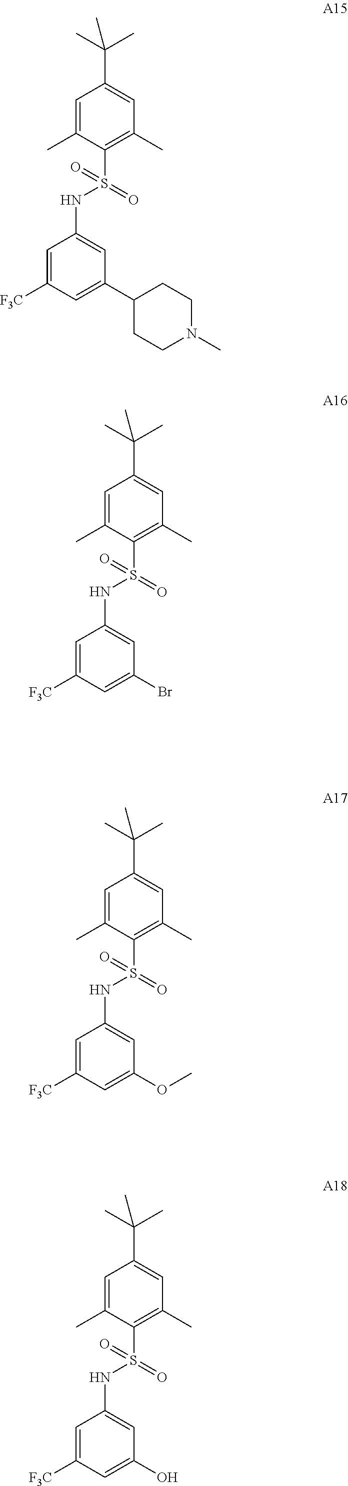 [SMILES: Cc1cc(C(C)(C)C)cc(C)c1S(=O)(=O)Nc1cc(C2CCN(C)CC2)cc(C(F)(F)F)c1 ; Cc1cc(C(C)(C)C)cc(C)c1S(=O)(=O)Nc1cc(Br)cc(C(F)(F)F)c1 ; COc1cc(NS(=O)(=O)c2c(C)cc(C(C)(C)C)cc2C)cc(C(F)(F)F)c1 ; Cc1cc(C(C)(C)C)cc(C)c1S(=O)(=O)Nc1cc(O)cc(C(F)(F)F)c1]

2,4,6-Triisopropyl-N-(5-methoxy-2-methyl-3-(trifluoromethyl)phenyl)-benzenesulfonamide A19. MS (ESI) m/z: 472.6 [M+H]⁺.

N-(5-Hydroxy-2-methyl-3-(trifluoromethyl)phenyl)-2,4,6-triisopropylbenzenesulfonamide A20. MS (ESI) m/z: 458.5 [M+H]⁺.

N-(3-Bromo-2-methyl-5-(trifluoromethyl)phenyl)-2,4,6-triisopropylbenzenesulfonamide A21. MS (ESI) m/z: 521.45 [M−99]⁺.

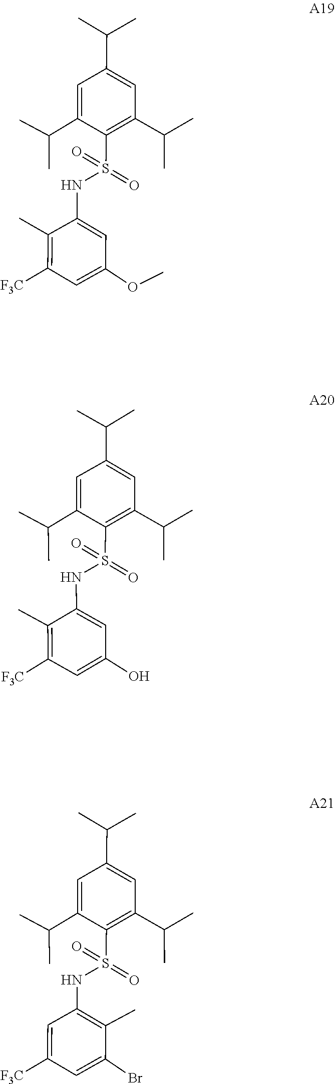

4-(Tert-butyl)-2,6-dimethyl-N-(3-(piperidin-4-yl)-5-(trifluoromethyl)phenyl)-benzenesulfonamide A22. MS (ESI) m/z: 469.6 [M+H]⁺.

N-(3-(3-Cyanopropoxy)-5-(trifluoromethyl)phenyl)-2,4,6-triisopropylbenzenesulfonamide A23.

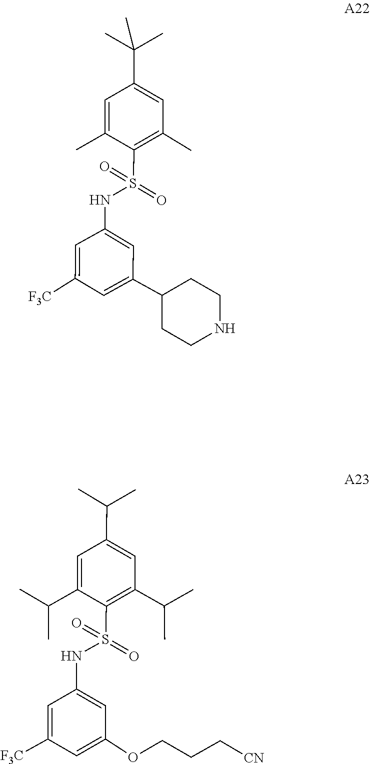 [SMILES: Cc1cc(C(C)(C)C)cc(C)c1S(=O)(=O)Nc1cc(C2CCNCC2)cc(C(F)(F)F)c1 ; CC(C)c1cc(C(C)C)c(S(=O)(=O)Nc2cc(OCCCC#N)cc(C(F)(F)F)c2)c(C(C)C)c1]

### Example 2

### Cell Proliferation Assay

Normal human lung fibroblasts were plated in a 6-well plate at 100,000 cells per well in 1 mL of DMEM supplemented with 10% FBS. The cells were incubated overnight at 37° C. under 5% CO₂. The next day, the cells were serum starved (medium was removed and 1 mL of fresh DMEM without serum was added). The cells were incubated overnight.

The cells were treated with a test compound in DMEM, 5% FBS in the presence of 10 ng/mL TGF-β and incubated for 24 h. The cells were lysed with an M-PER buffer. Alpha smooth muscle actin (α-SMA) levels were determined by Western blot and normalized to 0-tubulin levels using cell signaling antibodies. Blots were probed with secondary antibodies from LI-COR® Biosciences and read using a scanner from LI-COR® Biosciences. Quantification was also done using a software from LI-COR® Biosciences. The results are shown in Tables 1 to 4 below, where the control (Ctrl) is the fibroblasts in the absence of TGF-β and a test compound.

TABLE 1
Effect of compound A18, alone or in combination with
nintedanib (N) or pirfenidone (P), on α-SMA levels

| | n | Relative α-SMA |
|---|---|---|
| Ctrl | | 0.54 |
| TGF-β | 4 | 1 |
| A18 (5 μM) | 4 | 1.26 |
| A18 (10 μM) | 4 | 0.86 |
| A18 (20 μM) | 3 | 0.06 |
| N (5 μM) | 4 | 0.82 |
| N (5 μM) + A18 (5 μM) | 4 | 0.67 |
| N (5 μM) + A18 (10 μM) | 4 | 0.46 |
| N (5 μM) + A18 (20 μM) | 1 | 0.04 |
| P (200 μM) | 4 | 1.29 |
| P (200 μM) + A18 (5 μM) | 4 | 0.91 |
| P (200 μM) + A18 (10 μM) | 4 | 0.56 |
| P (200 μM) + A18 (20 μM) | 1 | 0.08 |

TABLE 2
Effect of compound B4, alone or in combination
with nintedanib (N), on α-SMA levels

| | n | Relative α-SMA |
|---|---|---|
| Ctrl | 7 | 0.56 |
| TGF-β | 7 | 1 |
| B4 (2 μM) | 6 | 0.95 |
| B4 (5 μM) | 3 | 0.69 |
| B4 (10 μM) | 3 | 0.15 |
| N (5 μM) | 6 | 0.92 |
| N (5 μM) + B4 (2 μM) | 6 | 0.60 |
| N (5 μM) + B4 (5 μM) | 3 | 0.52 |
| N (5 μM) + B4 (10 μM) | 3 | 0.14 |

TABLE 3
Effect of compound B5, alone or in combination with
nintedanib (N) or pirfenidone (P), on α-SMA levels

| | n | Relative α-SMA |
|---|---|---|
| Ctrl | 7 | 0.53 |
| TGF-β | 7 | 1 |
| B5 (5 μM) | 6 | 0.91 |
| B5 (10 μM) | 3 | 0.75 |
| B5 (20 μM) | 3 | 0.24 |
| N (5 μM) | 6 | 0.97 |
| N (5 μM) + B5 (5 μM) | 6 | 0.60 |
| N (5 μM) + B5 (10 μM) | 3 | 0.46 |
| N (5 μM) + B5 (20 μM) | 3 | 0.21 |
| P (200 μM) | 6 | 1.02 |
| P (200 μM) + B5 (5 μM) | 6 | 0.55 |
| P (200 μM) + B5 (10 μM) | 3 | 0.37 |
| P (200 μM) + B5 (20 μM) | 3 | 0.27 |

TABLE 4
Effect of compounds on α-SMA levels

| | Relative α-SMA | |
|---|---|---|
| Cmpd. | 5 μM | 20 μM |
| A1 | 0.98 | 0.37 |
| A2 | 1.05 | 0.23 |
| A3 | 0.79 | 0.73 |
| A4 | 1.30 | 0.46 |
| A5 | 0.95 | 0.21 |
| A6 | 1.08 | 0.05 |
| A7 | 1.60 | 0.33 |
| A8 | 0.88 | 0.33 |
| A9 | 1.05 | 0.78 |
| A10 | 0.69 | |
| A11 | 0.85 | |
| A12 | 0.37 | |
| A13 | 0.29 | |
| A14 | 0.53 | |
| A15 | 0.31 | |
| A16 | 0.35 | |
| A17 | 0.46 | |
| A18 | 0.57 | 0.28 |
| A19 | 0.56 | |
| A20 | 0.27 | |
| A21 | 0.45 | |
| A22 | 0.21 | 0.12 |

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

What is claimed is:

1. A method of treating, preventing, or ameliorating one or more symptoms of a fibrotic lung disease in a subject, comprising administering to the subject a therapeutically effective amount of a biarylsulfonamide and a therapeutically effective amount of an antifibrotic agent; wherein the biarylsulfonamide is a compound of Formula (I): 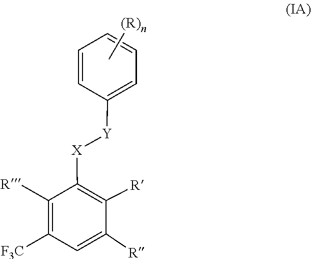
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof; wherein:
each R is independently deuterium, cyano, halo, nitro, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, heterocyclyl, —OR^{1a}, —C(O)OR^{1a}, —C(O)NR^{1b}R^{1c}, or —NR^{1b}R^{1c};
R′ and R″′ are each independently (i) hydrogen, deuterium, cyano, halo, or nitro;
(ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R″ is (i) deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c} or —S(O)₂NR^{1b}R^{1c};
each R^{1a}, R^{1b}, R^{1c}, and R^{1d} is independently hydrogen, deuterium, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or R^{1a} and R^{1c} together with the C and N atoms to which they are attached form heterocyclyl; or R^{1b} and R^{1c} together with the N atom to which they are attached form heterocyclyl;
X is —NH— and Y is —S(O)₂—; or X is —S(O)₂— and Y is —NH—; and
n is an integer of 2, 3, or 4;
wherein each alkyl, alkenyl, alkynyl, cycloalkyl, aryl, aralkyl, heteroaryl, and heterocyclyl is optionally substituted with one or more, in one embodiment, one, two, three, or four, substituents Q, wherein each Q is independently: (a) deuterium, cyano, halo, nitro, or oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is further optionally substituted with one or more, in one embodiment, one, two, three, or four, substituents Q^{a}; or (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, or —S(O)₂NR^{b}R^{c}, wherein each R^{a}, R^{b}, R^{c}, and R^{d} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl optionally substituted with one or more, in one embodiment, one, two, three, or four, substituents Q^{a};
wherein each Q^{a} is independently: (a) deuterium, cyano, halo, nitro, or oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; and (c) —C(O)R^{c}, —C(O)OR^{e}, —C(O)NR^{f}R^{g}, —C(O)SR^{e}, —C(NR^{c})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{c})NR^{f}R^{g}, —OC(S)R^{e}, —OC(S)OR^{e}, —OC(S)NR^{f}R^{g}, —OS(O)R^{e}, —OS(O)₂R^{1c}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{c}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{c}, —S(O)R^{1c}, —S(O)₂R^{1c}, —S(O)NR^{f}R^{g}, or —S(O)₂NR^{f}R^{g}; wherein each R^{c}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

2. A method of slowing the rate of decline in pulmonary function in a subject with a fibrotic lung disease or slowing the progression of a fibrotic lung disease in a subject, comprising administering to the subject a therapeutically effective amount of a biarylsulfonamide and a therapeutically effective amount of an antifibrotic agent; wherein the biarylsulfonamide is a compound of Formula (I): 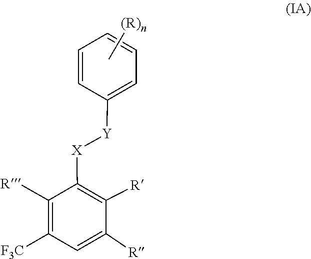
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof; wherein:
each R is independently deuterium, cyano, halo, nitro, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, heterocyclyl, —OR^{1a}, —C(O)OR^{1a}, —C(O)NR^{1b}R^{1c}, or —NR^{1b}R^{1c};
R′ and R″′ are each independently (i) hydrogen, deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c} or —S(O)₂NR^{1b}R^{1c};
R″ is (i) deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; with the proviso that R″ is not —CF₃;
each R^{1a}, R^{1b}, R^{1c}, and R^{1d} is independently hydrogen, deuterium, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or R^{1a} and R^{1c} together with the C and N atoms to which they are attached form heterocyclyl; or R^{1b} and R^{1c} together with the N atom to which they are attached form heterocyclyl;
X is —NH— and Y is —S(O)₂—; or X is —S(O)₂— and Y is —NH—; and
n is an integer of 2, 3, or 4;
wherein each alkyl, alkenyl, alkynyl, cycloalkyl, aryl, aralkyl, heteroaryl, and heterocyclyl is optionally substituted with one or more, in one embodiment, one, two, three, or four, substituents Q, wherein each Q is independently: (a) deuterium, cyano, halo, nitro, or oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is further optionally substituted with one or more, in one embodiment, one, two, three, or four, substituents Q^{a}; or (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, or —S(O)₂NR^{b}R^{c}, wherein each R^{a}, R^{b}, R^{c}, and R^{d} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl optionally substituted with one or more, in one embodiment, one, two, three, or four, substituents Q^{a};
wherein each Q^{a} is independently: (a) deuterium, cyano, halo, nitro, or oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{c})NR^{f}R^{g}, —OC(S)R^{e}, —OC(S)OR^{e}, —OC(S)NR^{f}R^{g}, —OS(O)R^{e}, —OS(O)₂R^{1c}, —OS(O)NR^{f}R^{g}, —OS(O)₂NR^{f}R^{g}, —NR^{f}R^{g}, —NR^{c}C(O)R^{h}, —NR^{c}C(O)OR^{f}, —NR^{c}C(O)NR^{f}R^{g}, —NR^{e}C(O)SR^{f}, —NR^{c}C(NR^{h})NR^{f}R^{g}, —NR^{e}C(S)R^{h}, —NR^{e}C(S)OR^{f}, —NR^{c}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{c}, —S(O)R^{1c}, —S(O)₂R^{1c}, —S(O)NR^{f}R^{g}, or —S(O)₂NR^{f}R^{g}; wherein each R^{c}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

3. The method of claim 1 or 2, wherein each R is independently deuterium, C₁₋₆ alkyl, C₃₋₁₀ cycloalkyl, C₆₋₁₄ aryl, or —OR^{1a}.

4. The method of claim 1 or 2, wherein each R is independently deuterium, C₁₋₄ alkyl, C₃₋₁₀ cycloalkyl, or —OC₁₋₆ alkyl; where each alkyl and cycloalkyl is optionally substituted with one or two substituents Q.

5. The method of claim 1 or 2, wherein each R is independently deuterium, C₁₋₆ alkyl, or C₃₋₁₀ cycloalkyl; wherein the alkyl and cycloalkyl are each optionally substituted with one or more substituents Q.

6. The method of claim 1 or 2, wherein each R is independently methyl, ethyl, propyl, butyl, or trifluoromethylcyclopropyl.

7. The method of claim 1 or 2, wherein each R is independently methyl, ethyl, isopropyl, tert-butyl, or 1-trifluoromethylcyclopropyl.

8. The method of any one of claims 1 to 7, wherein n is an integer of 2.

9. The method of any one of claims 1 to 7, wherein n is an integer of 3.

10. The method of claim 1 or 2, wherein the biarylsulfonamide is a compound of Formula (IIA): 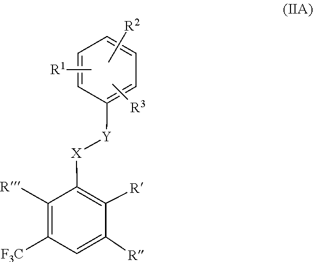
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof; wherein:
R¹ and R³ are each independently C₁₋₆ alkyl, C₂₋₆ alkenyl, C₂₋₆ alkynyl, or C₃₋₁₀ cycloalkyl, each of which is optionally substituted with one or more substituents Q; and
R₂ is (i) hydrogen or deuterium; or (ii) C₁₋₆ alkyl, C₂₋₆ alkenyl, C₂₋₆ alkynyl, or C₃₋₁₀ cycloalkyl, each of which is optionally substituted with one or more substituents Q.

11. The method of claim 10, wherein the biarylsulfonamide is a compound of Formula (V): 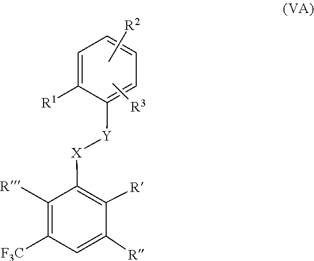
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof.

12. The method of claim 10, wherein the biarylsulfonamide is a compound of Formula (VIIIA): 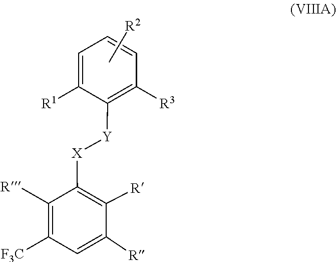
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof.

13. The method of claim 10, wherein the biarylsulfonamide is a compound of Formula (XIA): 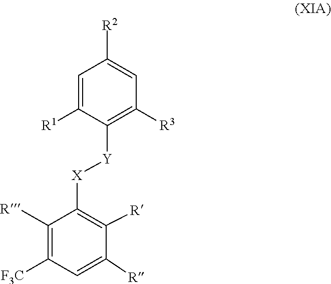
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof.

14. The method of any one of claims 10 to 13, wherein R¹ is C₁₋₆ alkyl or C₃₋₁₀ cycloalkyl, each optionally substituted with one or more substituents Q.

15. The method of any one of claims 10 to 13, wherein R¹ is C₁₋₆ alkyl optionally substituted with one or more substituents Q.

16. The method of any one of claims 10 to 13, wherein R¹ is C₁₋₄ alkyl.

17. The method of any one of claims 10 to 16, wherein R¹ is methyl, ethyl, or propyl.

18. The method of any one of claims 10 to 17, wherein R¹ is methyl, ethyl, or isopropyl.

19. The method of any one of claims 10 to 18, wherein R² is hydrogen, deuterium, C₁₋₆ alkyl, C₃₋₁₀ cycloalkyl, C₆₋₁₄ aryl, or —OR^{1a}; where the alkyl, cycloalkyl, and aryl are each optionally substituted with one or more substituents Q.

20. The method of any one of claims 10 to 18, wherein R² is hydrogen, deuterium, C₁₋₆ alkyl, C₃₋₁₀ cycloalkyl, C₆₋₁₄ aryl, or —OC₁₋₆ alkyl; where each alkyl and cycloalkyl are each optionally substituted with one or more substituents Q.

21. The method of any one of claims 10 to 18, wherein R² is hydrogen, deuterium, C₁₋₆ alkyl, or C₃₋₁₀ cycloalkyl, wherein the alkyl and cycloalkyl are each optionally substituted with one or more substituents Q.

22. The method of any one of claims 10 to 21, wherein R² is hydrogen, deuterium, propyl, butyl, or trifluoromethylcyclopropyl.

23. The method of any one of claims 10 to 22, wherein R² is hydrogen, deuterium, isopropyl, tert-butyl, or 1-trifluoromethylcyclopropyl.

24. The method of any one of claims 10 to 23, wherein R³ is C₁₋₆ alkyl or C₃₋₁₀ cycloalkyl, each optionally substituted with one or more substituents Q.

25. The method of any one of claims 10 to 23, wherein R³ is C₁₋₆ alkyl optionally substituted with one or more substituents Q.

26. The method of any one of claims 10 to 23, wherein R³ is C₁₋₄ alkyl.

27. The method of any one of claims 10 to 26, wherein R³ is methyl, ethyl, or propyl.

28. The method of any one of claims 10 to 27, wherein R³ is methyl, ethyl, or isopropyl.

29. The method of any one of claims 1 to 28, wherein R′ is hydrogen, deuterium, halo, or C₁₋₆ alkyl optionally substituted with one or more substituents Q.

30. The method of any one of claims 1 to 28, wherein R′ is hydrogen, deuterium, halo, or C₁₋₄ alkyl.

31. The method of any one of claims 1 to 30, wherein R′ is hydrogen, deuterium, chloro, bromo, or methyl.

32. The method of any one of claims 1 to 31, wherein R″ is halo, C₁₋₆ alkyl, heterocyclyl, —C(O)OR^{1a}, —C(O)NR^{1b}R^{1c}, —OR^{1a}, —NR^{1b}R^{1c}, —NR^{1a}C(O)R^{1d}, or —NR^{1a}C(O)OR^{1d}; wherein each alkyl and cycloalkyl is optionally substituted with one, two, or three substituents Q.

33. The method of any one of claims 1 to 31, wherein R″ is halo, C₁₋₆ alkyl, heterocyclyl, carboxy, —C(O)C₁₋₆ alkyl, —C(O)N(H)C₁₋₆ alkyl, hydroxyl, —OC₁₋₆ alkyl, amino, —N(H)C₁₋₆ alkyl, —N(C₁₋₆ alkyl)₂, —NHC(O)C₁₋₆ alkyl, or —NHC(O)OC₁₋₆ alkyl, wherein each alkyl and heterocyclyl is optionally substituted with one or two substituents, each independently selected from cyano, methyl, morpholinyl, amino, and dimethylamino.

34. The method of any one of claims 1 to 33, wherein R″ is (i) amino, bromo, carboxy, or hydroxyl; or (ii) methyl, propyl, piperidyl, methylcarboxy, butylaminocarbonyl, methoxy, ethoxy, propoxy, butoxy, diethylamino, acetamido, or butoxycarbonylamino, each of which is optionally substituted with one or two substituents, each independently selected from cyano, methyl, morpholinyl, amino, and dimethylamino.

35. The method of any one of claims 1 to 34, wherein R″ is bromo, methyl, 3-dimethylaminopropyl, 3-morpholin-4-ylpropyl, piperid-4-yl, 1-methylpiperid-4-yl, carboxy, methylcarboxy, butylaminocarbonyl, hydroxyl, methoxy, 2-dimethylaminoethoxy, 3-cyanopropoxy, 4-aminobutoxy, amino, diethylamino, acetamido, or tert-butoxycarbonylamino.

36. The method of any one of claims 1 to 35, wherein R′″ is hydrogen, deuterium, halo, or C₁₋₆ alkyl optionally substituted with one or more substituents Q.

37. The method of any one of claims 1 to 35, wherein R′″ is hydrogen, deuterium, halo, or C₁₋₄ alkyl.

38. The method of any one of claims 1 to 37, wherein R′″ is hydrogen, deuterium, chloro, bromo, or methyl.

39. The method of any one of claims 1 to 38, wherein X is —NH— and Y is —S(O)₂—.

40. The method of any one of claims 1 to 38, wherein X is —S(O)₂— and Y is —NH—.

41. The method of claim 1 or 2, wherein the biarylsulfonamide is:
2,4,6-triisopropyl-N-(3-methyl-5-(trifluoromethyl)phenyl)benzenesulfonamide A1;
methyl 3-(trifluoromethyl)-5-((2,4,6-triisopropylphenyl)sulfonamido)benzoate A2;
3-(trifluoromethyl)-5-((2,4,6-triisopropylphenyl)sulfonamido)benzoic acid A3;
N-butyl-3-(trifluoromethyl)-5-((2,4,6-triisopropylphenyl)sulfonamido)benzamide A4;
1 tert-butyl (3-(trifluoromethyl)-5-((2,4,6-triisopropylphenyl)sulfonamido)-phenyl)carbamate A5;
N-(3-amino-5-(trifluoromethyl)phenyl)-2,4,6-triisopropylbenzenesulfonamide A6;
N-(3-(trifluoromethyl)-5-((2,4,6-triisopropylphenyl)sulfonamido)phenyl)-acetamide A7;
N-(3-(diethylamino)-5-(trifluoromethyl)phenyl)-2,4,6-triisopropylbenzenesulfonamide A8;
4-(tert-butyl)-N-(3-(3-cyanopropoxy)-5-(trifluoromethyl)phenyl)-2,6-dimethylbenzenesulfonamide A9;
N-(3-(4-aminobutoxy)-5-(trifluoromethyl)phenyl)-4-(tert-butyl)-2,6-dimethylbenzenesulfonamide A10;
N-(3-(4-aminobutoxy)-5-(trifluoromethyl)phenyl)-2,4,6-triisopropylbenzenesulfonamide A11;
4-(tert-butyl)-N-(3-(2-(dimethylamino)ethoxy)-5-(trifluoromethyl)phenyl)-2,6-dimethylbenzenesulfonamide A12;
4-(tert-butyl)-N-(3-(3-(dimethylamino)propyl)-5-(trifluoromethyl)phenyl)-2,6-dimethylbenzenesulfonamide A13;
4-(tert-butyl)-2,6-dimethyl-N-(3-(3-morpholinopropyl)-5-(trifluoromethyl)-phenyl)benzenesulfonamide A14;
4-(tert-butyl)-2,6-dimethyl-N-(3-(1-methylpiperidin-4-yl)-5-(trifluoromethyl)-phenyl)benzenesulfonamide A15;
N-(3-bromo-5-(trifluoromethyl)phenyl)-4-(tert-butyl)-2,6-dimethyl-benzene-sulfonamide A16;
4-(tert-butyl)-N-(3-methoxy-5-(trifluoromethyl)phenyl)-2,6-dimethylbenzenesulfonamide A17;
4-(tert-butyl)-N-(3-hydroxy-5-(trifluoromethyl)phenyl)-2,6-dimethylbenzene-sulfonamide A18;
2,4,6-triisopropyl-N-(5-methoxy-2-methyl-3-(trifluoromethyl)phenyl)-benzenesulfonamide A19;
N-(5-hydroxy-2-methyl-3-(trifluoromethyl)phenyl)-2,4,6-triisopropylbenzenesulfonamide A20;
N-(3-bromo-2-methyl-5-(trifluoromethyl)phenyl)-2,4,6-triisopropylbenzenesulfonamide A21;
4-(tert-butyl)-2,6-dimethyl-N-(3-(piperidin-4-yl)-5-(trifluoromethyl)phenyl)-benzenesulfonamide A22;
N-(3-(3-cyanopropoxy)-5-(trifluoromethyl)phenyl)-2,4,6-triisopropylbenzenesulfonamide A23;
N-(3,5-bis(trifluoromethyl)phenyl)-2,4,6-triisopropylbenzenesulfonamide B1;
N-(2-chloro-3,5-bis(trifluoromethyl)phenyl)-2,4,6-triisopropyl-benzene-sulfonamide B2;
N-(2-bromo-3,5-bis(trifluoromethyl)phenyl)-2,4,6-triisopropyl-benzene-sulfonamide B3;
N-(2-methyl-3,5-bis(trifluoromethyl)phenyl)-2,4,6-triisopropyl-benzene-sulfonamide B4;
N-(3,5-bis(trifluoromethyl)phenyl)-4-(tert-butyl)-2,6-dimethylbenzene-sulfonamide B5;
N-(2-chloro-3,5-bis(trifluoromethyl)phenyl)-4-(tert-butyl)-2,6-dimethylbenzene-sulfonamide B6;
N-(2-methyl-3,5-bis(trifluoromethyl)phenyl)-4-(tert-butyl)-2,6-dimethylbenzene-sulfonamide B7;
N-(3,5-bis(trifluoromethyl)phenyl)-2,6-diethylbenzenesulfonamide B8;
N-(2-methyl-3,5-bis(trifluoromethyl)phenyl)-2,6-diethylbenzenesulfonamide B9;
3,5-bis(trifluoromethyl)-N-(2,4,6-triisopropylphenyl)benzenesulfonamide B10;
4-bromo-2-ethyl-N-(2-methyl-3,5-bis(trifluoromethyl)phenyl)benzene-sulfonamide C1;
2-chloro-5-trifluoromethyl-N-(2-methyl-3,5-bis(trifluoromethyl)phenyl)benzene-sulfonamide C2;
4-bromo-2-trifluoromethoxy-N-(2-methyl-3,5-bis(trifluoromethyl)phenyl)-benzenesulfonamide C3;
4-chloro-2-ethyl-N-(3,5-bis(trifluoromethyl)phenyl)benzenesulfonamide C₄;
methyl 4-(N-(3,5-bis(trifluoromethyl)phenyl)sulfamoyl)-3-methylbenzoate C₅;
4-(N-(2-methyl-3,5-bis(trifluoromethyl)phenyl)sulfamoyl)-3-methylbenzoic acid C₆;
3-methyl-4-(N-(2-methyl-3,5-bis(trifluoromethyl)phenyl)sulfamoyl)-N-pentylbenzamide C7;
N-(4-bromo-2-trifluoromethoxyphenyl)-3,5-bis(trifluoromethyl)benzene-sulfonamide C8;
N-(4-chloro-2-trifluoromethoxyphenyl)-3,5-bis(trifluoromethyl)benzene-sulfonamide C9;
N-(3,5-bis(trifluoromethyl)phenyl)-3,5-dimethyl-[1,1′-biphenyl]-4-sulfonamide C₁₀;
N-(4-cyclopropyl-2,6-dimethylphenyl)-3,5-bis(trifluoromethyl)benzene-sulfonamide C11;
N-(2,4,6-triethylphenyl)-3,5-bis(trifluoromethyl)benzenesulfonamide C₁₂;
4-methoxy-2,6-dimethyl-N-(2-methyl-3,5-bis(trifluoromethyl)phenyl)benzene-sulfonamide C13;
4-methoxy-2,6-dimethyl-N-(3,5-bis(trifluoromethyl)phenyl)benzenesulfonamide C14;
3-methyl-4-(N-(2-methyl-3,5-bis(trifluoromethyl)phenyl)sulfamoyl)benzoic acid C15;
N-(4-bromo-2,6-diethylphenyl)-3,5-bis(trifluoromethyl)benzenesulfonamide C₁₋₆;
N-(4-bromo-2,6-diisopropylphenyl)-3,5-bis(trifluoromethyl)benzenesulfonamide C17;
N-(4-cyclopropyl-2,6-diethylphenyl)-3,5-bis(trifluoromethyl)benzenesulfonamide C18;
methyl 4-((3,5-bis(trifluoromethyl)phenyl)sulfonamido)-3-methylbenzoate C₁₉;
(E)-N-(4-(4-hydroxybut-1-en-1-yl)-2,6-diisopropylphenyl)-3,5-bis(trifluoromethyl)benzenesulfonamide C20;
N-(4-(4-hydroxybutyl)-2,6-diisopropylphenyl)-3,5-bis(trifluoromethyl)benzene-sulfonamide C21;
N-(2-ethyl-4-(4-hydroxybutyl)-6-isopropylphenyl)-2-methyl-3,5-bis(trifluoromethyl)benzenesulfonamide C22;
4-amino-N-(3,5-bis(trifluoromethyl)phenyl)-2,6-dimethylbenzenesulfonamide C23;
4-(2-aminoethyl)-N-(3,5-bis(trifluoromethyl)phenyl)-2,6-dimethylbenzene-sulfonamide C24;
N-(3,5-bis(trifluoromethyl)phenyl)-2,6-dimethyl-4-propoxybenzenesulfonamide C₂₅;
N-(3,5-bis(trifluoromethyl)phenyl)-4-(3-cyanopropoxy)-2,6-dimethylbenzene-sulfonamide C26;
4-(4-aminobutoxy)-N-(3,5-bis(trifluoromethyl)phenyl)-2,6-dimethylbenzene-sulfonamide C27;
N-(4-(4-(N-(3,5-bis(trifluoromethyl)phenyl)sulfamoyl)-3,5-dimethylphenoxy)-butyl)acetamide C28;
N-(3,5-bis(trifluoromethyl)phenyl)-4-(tert-butyl)benzenesulfonamide C29; or
N-(3,5-bis(trifluoromethyl)phenyl)-4-(1-(trifluoromethyl)cyclopropyl)benzene-sulfonamide C30;
or a tautomer, a mixture of two or more tautomers, or an isotopic variant thereof; or a pharmaceutically acceptable salt, solvate, hydrate, or prodrug thereof.

42. The method of claim 1 or 2, wherein the biarylsulfonamide is 4-(tert-butyl)-2,6-dimethyl-N-(3-(1-methylpiperidin-4-yl)-5-(trifluoromethyl)-phenyl)benzenesulfonamide A15; 4-(tert-butyl)-N-(3-hydroxy-5-(trifluoromethyl)phenyl)-2,6-dimethylbenzene-sulfonamide A18; N-(2-methyl-3,5-bis(trifluoromethyl)phenyl)-2,4,6-triisopropyl-benzene-sulfonamide B4, or N-(3,5-bis(trifluoromethyl)phenyl)-4-(tert-butyl)-2,6-dimethylbenzene-sulfonamide B5, or a tautomer, a mixture of two or more tautomers, or an isotopic variant thereof; or a pharmaceutically acceptable salt, solvate, hydrate, or prodrug thereof.

43. The method of any one of claims 1 to 42, wherein the therapeutically effective amount of the biarylsulfonamide is ranging from about 0.1 to about 100 mg/kg/day.

44. The method of any one of claims 1 to 43, wherein the therapeutically effective amount of the biarylsulfonamide is ranging from about 1 to about 5,000 mg per day.

45. The method of any one of claims 1 to 44, wherein the biarylsulfonamide is administered orally.

46. The method of any one of claims 1 to 45, wherein the biarylsulfonamide is administered orally as a tablet or capsule.

47. The method of any one of claims 1 to 46, wherein the therapeutically effective amount of the antifibrotic agent is ranging from about 0.1 to about 100 mg/kg/day.

48. The method of any one of claims 1 to 47, wherein the therapeutically effective amount of the antifibrotic agent is ranging from about 1 to about 5,000 mg per day.

49. The method of any one of claims 1 to 48, wherein the antifibrotic agent is administered orally.

50. The method of any one of claims 1 to 49, wherein the antifibrotic agent is administered orally as a tablet or capsule.

51. The method of any one of claims 1 to 50, wherein the antifibrotic agent is nintedanib, or a pharmaceutically acceptable salt, solvate, or hydrate thereof.

52. The method of claim 51, wherein the antifibrotic agent is nintedanib ethanesulfonate.

53. The method of claim 51 or 52, wherein the therapeutically effective amount of the antifibrotic agent is ranging from about 200 to about 300 mg per day.

54. The method of any one of claims 51 to 53, wherein the antifibrotic agent is administered twice a day.

55. The method of any one of claims 51 to 54, wherein the antifibrotic agent is administered orally.

56. The method of any one of claims 51 to 55, wherein the antifibrotic agent is administered as a capsule.

57. The method of any one of claims 1 to 56, wherein the antifibrotic agent is pirfenidone, or a pharmaceutically acceptable salt, solvate, or hydrate thereof.

58. The method of claim 57, wherein the antifibrotic agent is pirfenidone.

59. The method of claim 57 or 58, wherein the therapeutically effective amount of the antifibrotic agent is ranging from about 500 to about 2,500 mg per day.

60. The method of any one of claims 57 to 59, wherein the antifibrotic agent is administered three times a day.

61. The method of any one of claims 57 to 60, wherein the antifibrotic agent is administered orally.

62. The method of any one of claims 57 to 61, wherein the antifibrotic agent is administered as a capsule or a tablet.

63. The method of any one of claims 1 to 62, wherein the fibrotic lung disease is interstitial lung disease.

64. The method of any one of claims 1 to 63, wherein the fibrotic lung disease is pulmonary fibrosis.

65. The method of claim 63 or 64, wherein the fibrotic lung disease is progressive fibrosing interstitial lung disease, a chronic fibrosing interstitial lung disease, or a chronic fibrosing interstitial lung disease with a progressive phenotype.

66. The method of claim 63 or 64, wherein the fibrotic lung disease is unclassifiable interstitial lung disease or progressive fibrosing unclassifiable interstitial lung disease.

67. The method of claim 63 or 64, wherein the fibrotic lung disease is an autoimmune interstitial lung disease, chronic hypersensitivity pneumonitis, sarcoidosis, myositis, Sjögren syndrome, coal workers pneumoconiosis, an idiopathic form of interstitial pneumonias, or idiopathic nonspecific interstitial pneumonia.

68. The method of claim 63 or 64, wherein the fibrotic lung disease is systemic sclerosis-interstitial lung disease.

69. The method of any one of claims 1 to 64, wherein the fibrotic lung disease is idiopathic pulmonary fibrosis.

70. The method of any one of claims 1 to 69, wherein the subject is a human.

71. A compound of Formula (IA): 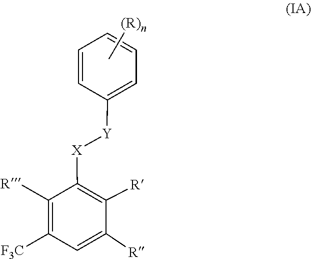
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof; wherein:
each R is independently deuterium, cyano, halo, nitro, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, heterocyclyl, —OR^{1a}, —C(O)OR^{1a}, —C(O)NR^{1b}R^{1c}, or —NR^{1b}R^{1c};
R′ and R″′ are each independently (i) hydrogen, deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c} or —S(O)₂NR^{1b}R^{1c};
R″ is (i) deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; with the proviso that R″ is not —CF₃;
each R^{1a}, R^{1b}, R^{1c}, and R^{1d} is independently hydrogen, deuterium, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or R^{1a} and R^{1c} together with the C and N atoms to which they are attached form heterocyclyl; or R^{1b} and R^{1c} together with the N atom to which they are attached form heterocyclyl;
X is —NH— and Y is —S(O)₂—; or X is —S(O)₂— and Y is —NH—; and
n is an integer of 2, 3, or 4;
wherein each alkyl, alkenyl, alkynyl, cycloalkyl, aryl, aralkyl, heteroaryl, and heterocyclyl is optionally substituted with one or more, in one embodiment, one, two, three, or four, substituents Q, wherein each Q is independently: (a) deuterium, cyano, halo, nitro, or oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is further optionally substituted with one or more, in one embodiment, one, two, three, or four, substituents Q^{a}; or (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, or —S(O)₂NR^{b}R^{c}, wherein each R^{a}, R^{b}, R′, and R^{d} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl optionally substituted with one or more, in one embodiment, one, two, three, or four, substituents Q^{a};
wherein each Q^{a} is independently: (a) deuterium, cyano, halo, nitro, or oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; and (c) —C(O)R^{e}, —C(O)OR^{e}, —C(O)NR^{f}R^{g}, —C(O)SR^{e}, —C(NR^{c})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{c})NR^{f}R^{g}, —OC(S)R^{e}, —OC(S)OR^{e}, —OC(S)NR^{f}R^{g}, —OS(O)R^{e}, —OS(O)₂R^{1c}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{c}, —S(O)R^{1c}, —S(O)₂R^{1c}, —S(O)NR^{f}R^{g}, or —S(O)₂NR^{f}R^{g}; wherein each R^{c}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

72. The compound of claim 71, wherein each R is independently deuterium, C₁₋₆ alkyl, C₃₋₁₀ cycloalkyl, C₆₋₁₄ aryl, or —OR^{1a}.

73. The compound of claim 71, wherein each R is independently deuterium, C₁₋₄ alkyl, C₃₋₁₀ cycloalkyl, or —OC₁₋₆ alkyl; where each alkyl and cycloalkyl is optionally substituted with one or two substituents Q.

74. The compound of claim 71, wherein each R is independently deuterium, C₁₋₆ alkyl, or C₃₋₁₀ cycloalkyl; wherein the alkyl and cycloalkyl are each optionally substituted with one or more substituents Q.

75. The compound of any one of claims 71 to 74, wherein each R is independently methyl, ethyl, propyl, butyl, or trifluoromethylcyclopropyl.

76. The compound of any one of claims 71 to 75, wherein each R is independently methyl, ethyl, isopropyl, tert-butyl, or 1-trifluoromethylcyclopropyl.

77. The compound of any one of claims 1 to 76, wherein n is an integer of 2.

78. The compound of any one of claims 1 to 76, wherein n is an integer of 3.

79. The compound of claim 71, wherein the biarylsulfonamide is a compound of Formula (IIA): 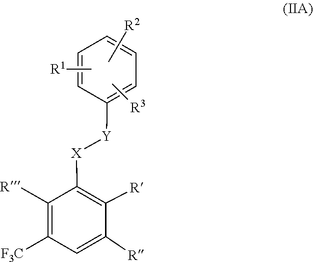
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof; wherein:
R¹ and R³ are each independently C₁₋₆ alkyl, C₂₋₆ alkenyl, C₂₋₆ alkynyl, or C₃₋₁₀ cycloalkyl, each of which is optionally substituted with one or more substituents Q; and
R₂ is (i) hydrogen or deuterium; or (ii) C₁₋₆ alkyl, C₂₋₆ alkenyl, C₂₋₆ alkynyl, or C₃₋₁₀ cycloalkyl, each of which is optionally substituted with one or more substituents Q.

80. The compound of claim 79, wherein the biarylsulfonamide is a compound of Formula (V): 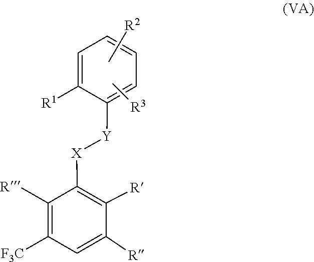
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof.

81. The compound of claim 79, wherein the biarylsulfonamide is a compound of Formula (VIIIA): 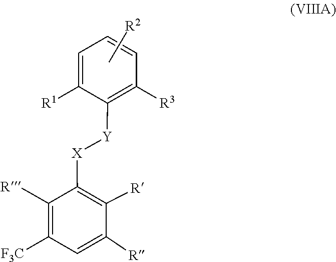
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof.

82. The compound of claim 79, wherein the biarylsulfonamide is a compound of Formula (XIA): 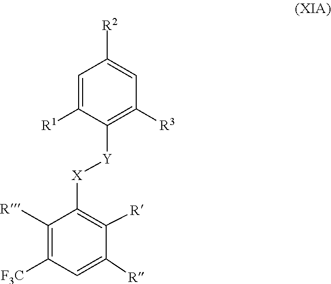
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof.

83. The compound of any one of claims 79 to 82, wherein R¹ is C₁₋₆ alkyl or C₃₋₁₀ cycloalkyl, each optionally substituted with one or more substituents Q.

84. The compound of any one of claims 79 to 82, wherein R¹ is C₁₋₆ alkyl optionally substituted with one or more substituents Q.

85. The compound of any one of claims 79 to 82, wherein R¹ is C₁₋₄ alkyl.

86. The compound of any one of claims 79 to 85, wherein R¹ is methyl, ethyl, or propyl.

87. The compound of any one of claims 79 to 86, wherein R¹ is methyl, ethyl, or isopropyl.

88. The compound of any one of claims 79 to 87, wherein R² is hydrogen, deuterium, C₁₋₆ alkyl, C₃₋₁₀ cycloalkyl, C₆₋₁₄ aryl, or —OR^{a}; where the alkyl, cycloalkyl, and aryl are each optionally substituted with one or more substituents Q.

89. The compound of any one of claims 79 to 87, wherein R² is hydrogen, deuterium, C₁₋₆ alkyl, C₃₋₁₀ cycloalkyl, C₆₋₁₄ aryl, or —OC₁₋₆ alkyl; where each alkyl and cycloalkyl are each optionally substituted with one or more substituents Q.

90. The compound of any one of claims 79 to 87, wherein R² is hydrogen, deuterium, C₁₋₆ alkyl, or C₃₋₁₀ cycloalkyl, wherein the alkyl and cycloalkyl are each optionally substituted with one or more substituents Q.

91. The compound of any one of claims 79 to 90, wherein R² is hydrogen, deuterium, propyl, butyl, or trifluoromethylcyclopropyl.

92. The compound of any one of claims 79 to 91, wherein R² is hydrogen, deuterium, isopropyl, tert-butyl, or 1-trifluoromethylcyclopropyl.

93. The compound of any one of claims 79 to 92, wherein R³ is C₁₋₆ alkyl or C₃₋₁₀ cycloalkyl, each optionally substituted with one or more substituents Q.

94. The compound of any one of claims 79 to 92, wherein R³ is C₁₋₆ alkyl optionally substituted with one or more substituents Q.

95. The compound of any one of claims 79 to 92, wherein R³ is C₁₋₄ alkyl.

96. The compound of any one of claims 79 to 95, wherein R³ is methyl, ethyl, or propyl.

97. The compound of any one of claims 79 to 96, wherein R³ is methyl, ethyl, or isopropyl.

98. The compound of any one of claims 79 to 97, wherein R′ is hydrogen, deuterium, halo, or C₁₋₆ alkyl optionally substituted with one or more substituents Q.

99. The compound of any one of claims 79 to 97, wherein R′ is hydrogen, deuterium, halo, or C₁₋₄ alkyl.

100. The compound of any one of claims 79 to 99, wherein R′ is hydrogen, deuterium, chloro, bromo, or methyl.

101. The compound of any one of claims 79 to 100, wherein R″ is halo, C₁₋₆ alkyl, heterocyclyl, —C(O)OR^{1a}, —C(O)NR^{1b}R^{1c}, —OR^{1a}, —NR^{1b}R^{1c}, —NR^{1a}C(O)R^{1d}, or —NR^{1a}C(O)OR^{1d}; wherein each alkyl and cycloalkyl is optionally substituted with one, two, or three substituents Q.

102. The compound of any one of claims 79 to 100, wherein R″ is halo, C₁₋₆ alkyl, heterocyclyl, carboxy, —C(O)C₁₋₆ alkyl, —C(O)N(H)C₁₋₆ alkyl, hydroxyl, —OC₁₋₆ alkyl, amino, —N(H)C₁₋₆ alkyl, —N(C₁₋₆ alkyl)₂, —NHC(O)C₁₋₆ alkyl, or —NHC(O)OC₁₋₆ alkyl, wherein each alkyl and heterocyclyl is optionally substituted with one or two substituents, each independently selected from cyano, methyl, morpholinyl, amino, and dimethylamino.

103. The compound of any one of claims 79 to 100, wherein R″ is (i) amino, bromo, carboxy, or hydroxyl; or (ii) methyl, propyl, piperidyl, methylcarboxy, butylaminocarbonyl, methoxy, ethoxy, propoxy, butoxy, diethylamino, acetamido, or butoxycarbonylamino, each of which is optionally substituted with one or two substituents, each independently selected from cyano, methyl, morpholinyl, amino, and dimethylamino.

104. The compound of any one of claims 79 to 103, wherein R″ is bromo, methyl, 3-dimethylaminopropyl, 3-morpholin-4-ylpropyl, piperid-4-yl, 1-methylpiperid-4-yl, carboxy, methylcarboxy, butylaminocarbonyl, hydroxyl, methoxy, 2-dimethylaminoethoxy, 3-cyanopropoxy, 4-aminobutoxy, amino, diethylamino, acetamido, or tert-butoxycarbonylamino.

105. The compound of any one of claims 79 to 104, wherein R′″ is hydrogen, deuterium, halo, or C₁₋₆ alkyl optionally substituted with one or more substituents Q.

106. The compound of any one of claims 79 to 104, wherein R′″ is hydrogen, deuterium, halo, or C₁₋₄ alkyl.

107. The compound of any one of claims 79 to 106, wherein R′″ is hydrogen, deuterium, chloro, bromo, or methyl.

108. The compound of any one of claims 79 to 107, wherein X is —NH— and Y is —S(O)₂—.

109. The compound of any one of claims 79 to 107, wherein X is —S(O)₂— and Y is —NH—.

110. The compound of claim 79, wherein the biarylsulfonamide is:
2,4,6-triisopropyl-N-(3-methyl-5-(trifluoromethyl)phenyl)benzenesulfonamide A1;
methyl 3-(trifluoromethyl)-5-((2,4,6-triisopropylphenyl)sulfonamido)benzoate A2;
3-(trifluoromethyl)-5-((2,4,6-triisopropylphenyl)sulfonamido)benzoic acid A3;
N-butyl-3-(trifluoromethyl)-5-((2,4,6-triisopropylphenyl)sulfonamido)benzamide A4;
1 tert-butyl (3-(trifluoromethyl)-5-((2,4,6-triisopropylphenyl)sulfonamido)-phenyl)carbamate A5;
N-(3-amino-5-(trifluoromethyl)phenyl)-2,4,6-triisopropylbenzenesulfonamide A6;
N-(3-(trifluoromethyl)-5-((2,4,6-triisopropylphenyl)sulfonamido)phenyl)-acetamide A7;
N-(3-(diethylamino)-5-(trifluoromethyl)phenyl)-2,4,6-triisopropylbenzenesulfonamide A8;
4-(tert-butyl)-N-(3-(3-cyanopropoxy)-5-(trifluoromethyl)phenyl)-2,6-dimethylbenzenesulfonamide A9;
N-(3-(4-aminobutoxy)-5-(trifluoromethyl)phenyl)-4-(tert-butyl)-2,6-dimethylbenzenesulfonamide A10;
N-(3-(4-aminobutoxy)-5-(trifluoromethyl)phenyl)-2,4,6-triisopropylbenzenesulfonamide A11;
4-(tert-butyl)-N-(3-(2-(dimethylamino)ethoxy)-5-(trifluoromethyl)phenyl)-2,6-dimethylbenzenesulfonamide A12;
4-(tert-butyl)-N-(3-(3-(dimethylamino)propyl)-5-(trifluoromethyl)phenyl)-2,6-dimethylbenzenesulfonamide A13;
4-(tert-butyl)-2,6-dimethyl-N-(3-(3-morpholinopropyl)-5-(trifluoromethyl)-phenyl)benzenesulfonamide A14;
4-(tert-butyl)-2,6-dimethyl-N-(3-(1-methylpiperidin-4-yl)-5-(trifluoromethyl)-phenyl)benzenesulfonamide A15;
N-(3-bromo-5-(trifluoromethyl)phenyl)-4-(tert-butyl)-2,6-dimethyl-benzene-sulfonamide A16;
4-(tert-butyl)-N-(3-methoxy-5-(trifluoromethyl)phenyl)-2,6-dimethylbenzenesulfonamide A17;
4-(tert-butyl)-N-(3-hydroxy-5-(trifluoromethyl)phenyl)-2,6-dimethylbenzene-sulfonamide A18;
2,4,6-triisopropyl-N-(5-methoxy-2-methyl-3-(trifluoromethyl)phenyl)-benzenesulfonamide A19;
N-(5-hydroxy-2-methyl-3-(trifluoromethyl)phenyl)-2,4,6-triisopropylbenzenesulfonamide A20;
N-(3-bromo-2-methyl-5-(trifluoromethyl)phenyl)-2,4,6-triisopropylbenzenesulfonamide A21;
4-(tert-butyl)-2,6-dimethyl-N-(3-(piperidin-4-yl)-5-(trifluoromethyl)phenyl)-benzenesulfonamide A22; or
N-(3-(3-cyanopropoxy)-5-(trifluoromethyl)phenyl)-2,4,6-triisopropylbenzenesulfonamide A23;
or a tautomer, a mixture of two or more tautomers, or an isotopic variant thereof; or a pharmaceutically acceptable salt, solvate, hydrate, or prodrug thereof.

111. A method of inhibiting transforming growth factor-β (TFG-β) in a subject, comprising administering to the subject in need thereof: (i) a therapeutically effective amount of a biarylsulfonamide and (ii) a therapeutically effective amount of an antifibrotic agent; wherein the biarylsulfonamide is a compound of Formula (IA): 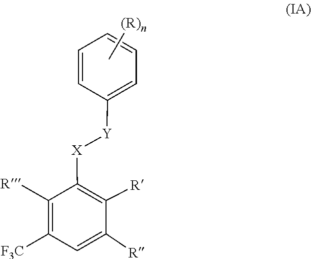
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof; wherein:
each R is independently deuterium, cyano, halo, nitro, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, heterocyclyl, —OR^{1a}, —C(O)OR^{1a}, —C(O)NR^{1b}R^{1c}, or —NR^{1b}R^{1c};
R′ and R″′ are each independently (i) hydrogen, deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c} or —S(O)₂NR^{1b}R^{1c};
R″ is (i) deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c} or —S(O)₂NR^{1b}R^{1c};
each R^{1a}, R^{1b}, R^{1c}, and R^{1d} is independently hydrogen, deuterium, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or R^{1a} and R^{1c} together with the C and N atoms to which they are attached form heterocyclyl; or R^{1b} and R^{1c} together with the N atom to which they are attached form heterocyclyl;
X is —NH— and Y is —S(O)₂—; or X is —S(O)₂— and Y is —NH—; and
n is an integer of 2, 3, or 4;
wherein each alkyl, alkenyl, alkynyl, cycloalkyl, aryl, aralkyl, heteroaryl, and heterocyclyl is optionally substituted with one or more, in one embodiment, one, two, three, or four, substituents Q, wherein each Q is independently: (a) deuterium, cyano, halo, nitro, or oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is further optionally substituted with one or more, in one embodiment, one, two, three, or four, substituents Q^{a}; or (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, or —S(O)₂NR^{b}R^{c}, wherein each R^{a}, R^{b}, R′, and R^{d} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl optionally substituted with one or more, in one embodiment, one, two, three, or four, substituents Q^{a};
wherein each Q^{a} is independently: (a) deuterium, cyano, halo, nitro, or oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; and (c) —C(O)R^{e}, —C(O)OR^{e}, —C(O)NR^{f}R^{g}, —C(O)SR^{e}, —C(NR^{c})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{c})NR^{f}R^{g}, —OC(S)R^{e}, —OC(S)OR^{e}, —OC(S)NR^{f}R^{g}, —OS(O)R^{e}, —OS(O)₂R^{1c}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{c}, —S(O)R^{1c}, —S(O)₂R^{1c}, —S(O)NR^{f}R^{g}, or —S(O)₂NR^{f}R^{g}; wherein each R^{c}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

112. A method of inhibiting transforming growth factor-β (TFG-β) in a cell, comprising contacting with (i) an effective amount of a biarylsulfonamide and (ii) an effective amount of an antifibrotic agent; wherein the biarylsulfonamide is a compound of Formula (IA): 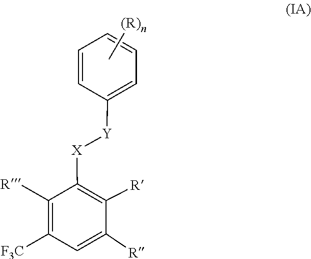
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof; wherein:
each R is independently deuterium, cyano, halo, nitro, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, heterocyclyl, —OR^{1a}, —C(O)OR^{1a}, —C(O)NR^{1b}R^{1c}, or —NR^{1b}R^{1c};
R′ and R″′ are each independently (i) hydrogen, deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R″ is (i) deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
each R^{1a}, R^{1b}, R^{1c}, and R^{1d} is independently hydrogen, deuterium, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or R^{1a} and R^{1c} together with the C and N atoms to which they are attached form heterocyclyl; or R^{1b} and R^{1c} together with the N atom to which they are attached form heterocyclyl;
X is —NH— and Y is —S(O)₂—; or X is —S(O)₂— and Y is —NH—; and
n is an integer of 2, 3, or 4;
wherein each alkyl, alkenyl, alkynyl, cycloalkyl, aryl, aralkyl, heteroaryl, and heterocyclyl is optionally substituted with one or more, in one embodiment, one, two, three, or four, substituents Q, wherein each Q is independently: (a) deuterium, cyano, halo, nitro, or oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is further optionally substituted with one or more, in one embodiment, one, two, three, or four, substituents Q^{a}; or (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, or —S(O)₂NR^{b}R^{c}, wherein each R^{a}, R^{b}, R^{c}, and R^{d} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl optionally substituted with one or more, in one embodiment, one, two, three, or four, substituents Q^{a};
wherein each Q^{a} is independently: (a) deuterium, cyano, halo, nitro, or oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; and (c) —C(O)R^{c}, —C(O)OR^{e}, —C(O)NR^{f}R^{g}, —C(O)SR^{e}, —C(NR^{c})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{c})NR^{f}R^{g}, —OC(S)R^{e}, —OC(S)OR^{e}, —OC(S)NR^{f}R^{g}, —OS(O)R^{e}, —OS(O)₂R^{1c}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{c}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{c}, —S(O)R^{1c}, —S(O)₂R^{1c}, —S(O)NR^{f}R^{g}, or —S(O)₂NR^{f}R^{g}; wherein each R^{c}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.
